(19) United States
(12) Patent Application Publication
(10) Pub. No.: US 2017/0202939 A1
(43) Pub. Date: Jul. 20, 2017

(54) PERSONALIZED CANCER VACCINES AND METHODS THEREFOR

(71) Applicant: Washington University, Saint Louis, MO (US)

(72) Inventors: Beatriz CARRENO, Philadelphia, PA (US); Gerald LINETTE, Philadelphia, PA (US); Elaine MARDIS, Troy, IL (US); Vincent MAGRINI, Saint Louis, MO (US)

(21) Appl. No.: 15/458,149

(22) Filed: Mar. 14, 2017

Related U.S. Application Data
(63) Continuation of application No. PCT/US15/49836, filed on Sep. 11, 2015.
(60) Provisional application No. 62/141,602, filed on Apr. 1, 2015.
(60) Provisional application No. 62/050,195, filed on Sep. 14, 2014.

(51) Int. Cl. A61K 39/00; G01N 33/569; C12N 5/0784; C12Q 1/68

(52) U.S. Cl. CPC A61K 39/0011; C12Q 1/6881; G01N 33/56977; C12N 5/0639; A61K 2039/5154; A61K 2039/572; C12N 2501/998

(57) ABSTRACT

Methods of cane r treatment based, on personalized vaccines are disclosed. Individual amino acid substitutions from tumors are revealed using whole genome sequencing, and identified as neoantigens silico. Peptide sequences are then tested in vitro for ability to bind HLA molecules and to be presented to CD8+ T-cells. A vaccine is formed using neoantigen peptides and an adjuvant or dendritic cells (DC) autologous to a subject. In the latter, autologous DC are matured and contacted with the neoantigen peptides. The DC are then administered to the subject. PBMC are then obtained from the subject, and CD8+ T cells specific to the neoantigens are cultured and enriched. Enriched T-cells are then administered to the subject to treat cancer. Treatment resulted in tumor regression in mice bearing human melanomas, and complete or partial responses were observed in human patients.

### REFERENCE TO PRIOR APPLICATIONS

This application claims the benefit of and priority to PCT application PCT/US15/49836, filed Sep. 11, 2015. which claims benefit of and priority to U.S. Provisional Application 62/050,195 filed on Sep. 14, 2014. PCT/US15/49836 also claims the benefit of and priority to U.S. Provisional Application 62/141,602 filed Apr. 1, 2015. Each of these applications are hereby incorporated by reference, each in their entirety.

### STATEMENT OF GOVERNMENT SUPPORT

This invention was made with government support under CA179695 awarded by the National Institutes of Health. The government has certain rights in the invention.

### REFERENCE TO A SEQUENCE LISTING

The Sequence Listing, which is a part of the present disclosure, includes a text file comprising primer nucleotide and/or amino acid sequences of the present invention. The subject matter of the Sequence Listing is incorporated herein by reference in its entirety. The information recorded in computer readable form is identical to the written sequence listing.

### INTRODUCTION

The incidence of malignant melanoma continues to rise worldwide. The number of new cases, in the US for 2012 is estimated to be 76,250 (8.6% increase compared to 2011) (Siegel, R., et al., Cancer statistics, 62, 10-29 2012). Despite recent advances in the treatment of metastatic melanoma with ipilimumab (anti-CTLA-4 antibody) and vemurafenib (BRAF V600E inhibitor), this disease remains an incurable malignancy with an expected survival of 12-14 months (Hodi, F. S., et al., N. Engl. J. Med. 363, 711-723, 2010; Chapman, P. B., et al., N. Engl. J. Med. 364, 2507-2516, 2011). Thus, metastatic melanoma represents a disease area of unmet medical need. Melanoma is distinguished for its association with early in life UV-light exposure, high mutational rate, and the ability to induce spontaneous anti-tumor immunity (Lennerz, V., et al., Proc. Nat'l. Acad. Sci. USA 102, 16013-16018, 2005; Garibyan, L., et al., Curr. Oncol. Rep. 12, 319-326, 2010; Pleasance, E. D. et al., Nature 463, 191-196, 2010; Berger, M. F., et al., Nature 485, 502-506, 2012; Hodis, E., et al., Cell 150, 251-263, 2012). The modest, yet reproducible, clinical activity of ipilimumab seen in patients with advanced melanoma provides strong evidence that immune targeting confers therapeutic benefit in this disease. Investigational cancer vaccines as well as adoptive T cell therapies while more technically demanding are now beginning to show efficacy in early phase clinical trials (Rosenberg, S. A. Science Translational Medicine 4, 127ps128, 2012).

However, a critical barrier facing investigators developing these cellular therapies is the paucity of validated melanoma antigens. New strategies are needed to identify patient-specific (unique) tumor antigens, which can serve as targets for immune intervention. Identification of the entire spectrum of unique antigens at the single tumor/patient level has been viewed historically as an unattainable goal.

### SUMMARY

The present inventors have developed anti-cancer vaccines, methods of constructing vaccines, methods of their use, and methods of identifying neoantigens create personalized vaccines to treat cancer. In various embodiments, the present teachings provide methods for identification of tumor-specific neoantigens and their incorporation in a vaccine, and adoptive T cell therapy for the treatment of cancers such as, without limitation, melanoma and lung cancer. Various embodiments involve patient-specific identification of tumor neo-antigens. In various configurations, such tumor neo-antigens, such as those arising during neoplastic transformation, can elicit T cell immunity capable of protecting the host from cancer progression. In various embodiments, the present teachings make use of next-generation sequencing technology, human leukocyte antigens (HLA) class I binding/stability prediction algorithms and in vitro assays to identify personalized tumor neoantigens. In various embodiments, these technologies can be incorporated into a vaccine/adoptive T cell therapy for treatment of cancer.

In some embodiments, the present teachings include strategies for personalized neoantigen-specific adoptive T cell therapy. In various aspects, DNA isolated from tumor and matched peripheral blood mononuclear cells (PBMC) can be subjected to exome sequencing to identify tumor somatic missense mutations. In some embodiments, RNA isolated from a tumor can be used for transcriptome analysis to identify those somatic mutations that are expressed. In some aspects, results can show that in cancers such as melanoma and lung cancer, a high number of missense mutations (>200) can be identified per tumor genome. In some embodiments, a combination of major histocompatibility complex (MHC) class I binding and stability prediction algorithms can be used to identify candidate neo-antigens among missense mutations, and expressed candidate neo-antigens can be selected for, peptide manufacturing. Biochemical and cellular assays can be performed to established binding and presentation of neo antigen-encoding peptides. Experimentally validated peptides can be selected for incorporation in a dendritic cell (DC) vaccine as described in Carreno, B. M., et al., J. Clin. Invest. 123, 3383-3394, 2013; after 3 vaccine doses patients can be subjected to apheresis and CD8+ T cells can be isolated from PBMC. These T cells can be expanded in an antigen-specific manner using a 2 step procedure as described in Carreno, B. M., et al., J. Immunology 188, 5839-5849, 2012. In various configurations, the 2 step procedure can take 10-30 days, such as, without limitation, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days or 30 days for completion and can yield>10⁴ fold antigen-specific T cell expansions. In various configurations, expanded neo-antigen specific T cells, can be infused into pre-conditioned patients as adoptive T cell therapy, by, for example, methods described by Linette, G. P. et al., Clin. Cancer Res. 11, 7692-7699, 2005.

In various configurations, the present teachings include a series of analytical steps for identification of neo-antigens from somatic tumor missense mutations, as illustrated in FIG. 1. In various embodiments, DNA isolated from tumor and matched PBMC can be subjected to exome sequencing in order to identify tumor somatic missense mutations. For example, in melanoma and lung cancer high number of missense mutations (>200) can be identified per tumor genome. Prediction algorithms such as, without limitation, PePSSI (Bui, H. H., et al., Proteins 63, 43-52, 2006) can be used for the identification of candidate tumor neo-antigen epitopes presented in the context of the patient's HLA class I molecules. In various configurations, analysis of tumor transcriptome data can be used for the selection, among predicted candidates, of those epitopes that are expressed by the tumor.

Various embodiments of the present teachings include the following aspects: In some embodiments, a method of treating a cancer in a subject in need thereof can comprise: providing a neoantigen peptide encoded in DNA of a tumor of the subject, wherein the neoantigen peptide can consist of from 8 to 13 amino acids; transfecting at least one HLA class I positive cell with at least one tandem minigene construct that can comprise at least one sequence that can encode the at least one neoantigen; identifying a complex that can comprise the at least one HLA molecule and the at least one neoantigen peptide produced by the at least one HLA class I positive cell; forming a vaccine that can comprise the at least one neoantigen; and administering the vaccine to the subject, wherein at least one tumor cell of the cancer can comprise at least one polypeptide which can comprise at least one amino acid substitution. In some configurations, the at least one neoantigen peptide can consist of from 9 to 11 amino acids. In some configurations, the at least one neoantigen peptide can consist of 9 amino acids. In various configurations, the at least one neoantigen peptide can consist of 8, 9, 10, 11, 12, or 13 amino acids. In some configurations, the at least one neoantigen peptide can bind in silico to an HLA class I molecule with a stability>2 h. In some configurations, the at least one neoantigen peptide can bind in silico to an HLA class I molecule an affinity of <500 nM. In some configurations, the at least one neoantigen peptide can bind in silico to an HLA class I molecule with an affinity of <250 nM. In various configurations, the at least one neoantigen peptide can bind in silico to an HLA Class I molecule with an affinity of <550 nM, <500 nM, <450 nM, <400 nM, <350 nM, <300 nM, <250 nM, or <200 nM. In various configurations, the at least one neoantigen peptide can bind in vitro to an HLA class I molecule with an affinity of <4.7 log (IC₅₀, nM), <4.6 log (IC₅₀, nM), <4.5 log (IC₅₀, nM), <4.4 log (IC₅₀, nM), <4.3 log (IC₅₀, nM), <4.2 log (IC₅₀, nM), <4.1 log (IC₅₀, nM), <4.0 log nM), <3.9 log (IC₅₀, nM), <3.8 log (IC₅₀, nM), or <3.7 log (IC₅₀, nM). In some configurations, the at least one neoantigen peptide can bind in vitro to an HLA class I molecule with an affinity of <4.7 log (IC₅₀, nM). In some configurations, the at least one neoantigen peptide can bind in vitro to an HLA class I molecule with an affinity of <3.8 log (IC₅₀, nM). In some configurations, the at least one neoantigen peptide can bind in vitro to an HLA class I molecule with an affinity of <3.7 log (IC₅₀, nM). In some configurations, the at least one neoantigen peptide can bind in vitro to an HLA class I molecule with an affinity of <3.2 log (IC₅₀, nM). In some configurations, the vaccine can comprise at least seven neoantigen peptides. In various configurations, the HLA class I molecules can be selected from the group consisting of HLA-A*01:01, HLA-B*07:02, HLA-A*02:01, HLA-B*07:03, HLA-A*02:02, HLA-B*08:01, HLA-A*02:03, HLA-B*15:01, HLA-A*02:05, HLA-B*15:02, HLA-A*02:06, HLA-B*15:03, HLA-A*02:07, HLA-B*15:08, HLA-A*03:01, HLA-B*15:12, HLA-A*11:01, HLA-B*15:16, HLA-A*11:02, HLA-B*15:18, HLA-A*24:02, HLA-B*27:03, HLA-A*29:01, HLA-B*27:05, HLA-A*29:02, HLA-B*27:08, HLA-A*34:02, HLA-B*35:01, HLA-A*36:01, HLA-B*35:08, HLA-B*42:01, HLA-B*53:01, HLA-B*54:01, HLA-B*56:01, HLA-B*56:02, HLA-B*57:01, HLA-B*57:02, HLA-B*57:03, HLA-B*58:01, HLA-B*67:01, and HLA-B*81:01. In some configurations, the HLA class I molecules can be HLA-A*02:01 molecules. In some configurations, the HLA class I molecules can be HLA-A*11:01 molecules. In some configurations, the HLA class I molecules can be HLA-B*08:01 molecules. In some configurations, the at least one HLA class I positive cell can be at least one melanoma cell. In various configurations, the at least one melanoma cell can be selected from the group consisting of DM6 cell and an A375 cell. In some configurations, the tandem minigene can further comprise a ubiquitination signal and two mini-gene controls. In configurations where the neoantigens bind HLA-A*2:01 molecules, the tandem minigene can further comprise a ubiquitination signal and two mini-gene controls that encode HLA-A*02:01 peptides G280 and WNV SVG9. In various configurations, the cancer can be selected from the group consisting of skin cancer, lung cancer, bladder cancer, colorectal cancer, gastrointestinal cancer, esophageal cancer, gastric cancer, intestinal cancer, breast cancer, and a cancer caused by a mismatch repair deficiency. In various configurations, the skin cancer can be selected from the group consisting of basal cell carcinoma, squamous cell carcinoma, merkel cell carcinoma, and melanoma. In some configurations, the cancer can be a melanoma. In some configurations, the forming a vaccine can comprise: providing a culture comprising dendritic cells obtained from the subject; and contacting the dendritic cells with the at least one neoantigen peptide, thereby forming dendritic cells comprising the at least one neoantigen peptide. In some configurations, the forming a vaccine can further comprise maturing the dendritic cells. In some configurations, the maturing the dendritic cells can comprise administering CD40L and IFNγ. In various configurations, the maturing the dendritic cells can further comprise administering TLR agonist. In various configurations, the maturing the dendritic cells can further comprise administering a TLR3 agonist. In various configurations, the maturing the dendritic cells can further comprise administering a TLR8 agonist. In various configurations, the maturing the dendritic cells can further comprise administering TLR3 and TLR8 agonists. In various configurations, the maturing the dendritic cells can further comprise administering poly I:C and R848. In some configurations, the forming a vaccine can further comprise: administering to the subject the dendritic cells comprising the at least one neoantigen peptide; obtaining a population of CD8+ T cells from a peripheral blood sample from the subject, wherein the CD8+ cells recognize the at least one neoantigen; and expanding the population of CD8+ T cells that recognize the neoantigen. In some configurations, the forming a vaccine can further comprise administering to the subject the expanded CD8+ T cells. In various configurations, the forming a vaccine can comprise combining the neoantigen peptide with a pharmaceutically acceptable adjuvant.

In some embodiments, a method of treating a cancer in a subject in need thereof, can comprise: a) providing a sample of a tumor from a subject; b) performing exome sequencing on the sample to identify one or more amino acid substitutions comprised by the tumor exome; c) performing transcriptome sequencing on the sample to verify expression of the amino acid substitutions identified in b); and d) selecting at least one candidate neoantigen peptide sequence from amongst the amino acid substitutions identified in c) according to the following criteria: i) Exome VAF>10%; ii) Transcription VAF>10%; iii) Alternate reads>5; iv) FPKM>1. v) binds in silico to an HLA class I molecule with an affinity of <500 nM and a stability>2 h; e) performing an in vitro HLA class I binding assay; f) selecting at least one candidate neoantigen peptide sequence from amongst the amino acid substitutions identified in d) that bind HLA class one molecules with an affinity of <4.7 log (IC₅₀, nM) in the assay performed in e); g) transfecting at least one HLA class I positive cell with at least one tandem minigene construct which can comprise at least one sequence encoding the at least one neoantigen; identifying a complex comprising the at least one HLA molecule and the at least one neoantigen peptide produced by the at least one HLA class I positive cell; i) forming a vaccine that can comprise the at least one neoantigen; and j) administering the vaccine to the subject, wherein at least one tumor cell of the cancer can comprise at least one polypeptide comprising the one or more amino acid substitutions. In some configurations, the Exome VAF can be ≧30%. In some configurations, the Exome VAF can be ≧40%. In some configurations, the Exome VAF can be ≧50%. In various configurations, the in vitro HLA class I binding assay can be selected from the group consisting of a T2 assay and a fluorescence polarization assay.

In some embodiments, a method of treating cancer in a subject in need thereof can comprise: a) providing a sample of a tumor from a subject; b) performing exome sequencing on the sample to identify amino acid substitutions comprised by the tumor exome; c) performing transcriptome sequencing on the sample to verify expression of the amino acid substitutions identified in b); d) performing a fluorescence polarization binding assay or a T2 assay of amino acid substitutions identified in c) to an HLA class I molecule; e) selecting at least one candidate neoantigen from amongst the amino acid substitutions identified in d) according to the following criteria: i) Exome variant allele fraction (VAF)>10%; ii) Transcriptome (seq capture data) VAF>10%; is iii) Alternate reads>5; iv) fragments per kilobase of exon per million fragments mapped (FPKM) (>1; v) Peptides comprise 9-11 amino acids; vi) Peptides are predicted in silico to bind to any HLA class I allele that meet the following criteria: A) Predicted MHC binding<250 nM; B) Predicted MHC stability>2 h; vii) MHC binding<3.2 log [IC₅₀, nM] in fluorescence polarization binding assay; f) transfecting at least one HLA class I positive cell line such as a melanoma cell line with at least one tandem minigene construct comprising at least one sequence encoding the at least one candidate neoantigen identified in e); g) extracting from the at least one HLA class I positive cell line one or more HLA class I complexes comprising a HLA class I molecule and the one or more neoantigen peptides; h) identifying the sequence of at least one neoantigen peptide comprised by the soluble HLA class I complex using reverse phase HPLC and LC/MS; i) contacting dendritic cells obtained from the subject with the at least one neoantigen peptide of sequence identified in h), thereby forming dendritic cells comprising the at least one neoantigen peptide; j) administering to the subject the dendritic cells comprising the at least one neoantigen peptide; k) obtaining CD8+ T cells from a peripheral blood sample from the subject; l) enriching the CD8+ T cells that recognize the at least one neoantigen; m) administering to the subject the enriched CD8+ T cells. In some configurations of the present teachings, the HLA class I molecules can be selected from the group consisting of HLA-A*01:01, HLA-B*07:02, HLA-A*02:01, HLA-B*07:03, HLA-A*02:02, HLA-B*08:01, HLA-A*02:03, HLA-B*15:01, HLA-A*02:05, HLA-B*15:02, HLA-A*02:06, HLA-B*15:03, HLA-A*02:07, HLA-B*15:08, HLA-A*03:01, HLA-B*15:12, HLA-A*11:01, HLA-B*15:16, HLA-A*11:02, HLA-B*15:18, HLA-A*24:02, HLA-B*27:03, HLA-A*29:01, HLA-B*27:05, HLA-A*29:02, HLA-B*27:08, HLA-A*34:02, HLA-B*35:01, HLA-A*36:01, HLA-B*35:08, HLA-B*42:01, HLA-B*53:01, HLA-B*54:01, HLA-B*56:01, HLA-B*56:02, HLA-B*57:01, HLA-B*57:02, HLA-B*57:03, HLA-B*58:01, HLA-B*67:01, and HLA-B*81:01. In some configurations, the HLA class I molecules can be HLA-A*02:01 molecules. In some configurations, the HLA class I molecules can be HLA-A*11:01 molecules. In some configurations, the HLA class I molecules can be HLA-B*08:01 molecules. In various configurations, the melanoma cell line can be selected from the group consisting of DM6 and A375. In some configurations, the tandem minigene can further comprise a ubiquitination signal and two mini-gene controls. In configurations where the HLA-A molecules are HLA-A*02:01 molecules, the two mini-gene controls can encode G280 and WNV SVG9 peptides. In some configurations, the cancer can be a melanoma. In various configurations, the melanoma is a metastatic melanoma.

In some configurations, as many as 600 amino acid substitutions can be identified from any given tumor. In some configurations, each of these amino acid substitutions can be analyzed for predicted binding to HLA-A class I molecules. In various configurations, at least 1, at least 2, at least 3, at least 4, at least 5, at least 6, at least 7, at least 8, at least 9, at least 10, at least 11, at least 12, at least 13, at least 15, at least 16, at least 17, at least 18, at least 19, at least 20, at least 21, at least 22, at least 23, at least 24, at least 25, at least 26, at least 27, at least 28, at least 29, at least 30, at least 11, at least 32, at least 33, at least 34, at least 35, at least 36, at least 37, at least 38, at least 39, at least 40, at least 41, at least 42, at least 43, at least 44, at least 45, at least 46, at least 47, at least 48, at least 49 or at least 50 candidate neoantigens can be expressed in a tumor. In some configurations, at least 1, at least 2, at least 3, at least 4, at least 5, at least 6, at least 7, at least 8, at least 9, at least 10, at least 11, at least 12, at least 13, at least 15, at least 16, at least 17, at least 18, at least 19, at least 20, at least 21, at least 22, at least 23, at least 24, at least 25, at least 26, at least 27, at least 28, at least 29, at least 30, at least 31, at least 32, at least 33, at least 34, at least 35, at least 36, at least 37, at least 38, at least 39, at least 40, at least 41, at least 42, at least 43, at least 44, at least 45, at least 46, at least 47, at least 48, at least 49 or at least 50 candidate neoantigens can be selected to test their presentation to T cells. In some configurations, at least 1, at least 2, at least 3, at least 4, at least 5, at least 6, at least 7, at least 8, at least 9, at least 10, at least 11, at least 12, at least 13, at least 15, at least 16, at least 17, at least 18, at least 19, at least 20, at least 21, at least 22, at least 23, at least 24, at least 25, at least 26, at least 27, at least 28, at least 29, at least 30, at least 31, at least 32, at least 33, at least 34, at least 35, at least 36, at least 37, at least 38, at least 39, at least 40, at least 41, at least 42, at least 43, at least 44, at least 45, at least 46, at least 47, at least 48, at least 49 or at least 50 candidate neoantigens can be selected for incorporation into a vaccine. In some configurations, the tandem minigenes can comprise at least 1, at least 2, at least 3, at least 4, at least 5, at least 6, at least 7, at least 8, at least 9, at least 10, at least 11, at least 12, at least 13, at least 15, at least 16, at least 17, at least 18, at least 19, at least 20, at least 21, at least 22, at least 23, at least 24, at least 25, at least 26, at least 27, at least 28, at least 29, at least 30, at least 31, at least 32, at least 33, at least 34, at least 35, at least 36, at least 37, at least 38, at least 39, at least 40, at least 41, at least 42, at least 43, at least 44, at least 45, at least 46, at least 47, at least 48, at least 49 or at least 50 candidate neoantigen sequences. In some configurations, the dendritic cells can comprise at least 1, at least 2, at least 3, at least 4, at least 5, at least 6, at least 7, at least 8, at least 9, at least 10, at least 11, at least 12, at least 13, at least 15, at least 16, at least 17, at least 18, at least 19, at least 20, at least 21, at least 22, at least 23, at least 24, at least 25, at least 26, at least 27, at least 28, at least 29, at least 30, at least 31, at least 32, at least 33, at least 34, at least 35, at least 36, at least 37, at least 38, at least 39, at least 40, at least 41, at least 42, at least 43, at least 44, at least 45, at least 46, at least 47, at least 48, at least 49 or at least 50 neoantigen peptides. In some embodiments, the personalized neoantigen therapy can be paired with other forms of cancer therapy such as, but without limitation, chemotherapy. In some configurations, the chemotherapy can comprise ipilimumab and/or vemurafenib.

In some embodiments, the present teachings include a neoantigen peptide encoded in DNA of a tumor of the subject for use in the treatment of a cancer, wherein the neoantigen peptide consists of from 8 to 13 amino acids, binds in silico to an HLA class I molecule with an affinity of <500 nM and a stability>2 h and binds in vitro to an HLA class I molecule with an affinity of <4.7 log (IC₅₀, nM).

In various embodiments of the invention, it includes the following aspects;
1. A method of treating a cancer in a subject in need thereof, comprising: providing a neoantigen peptide encoded in DNA of a tumor of the subject, wherein the neoantigen peptide consists of from 8 to 13 amino acids, binds in silico to an HLA class I molecule with an affinity of <500 nM and a stability>2 h and binds in vitro to an HLA class I molecule with an affinity of <4.7 log (IC50, nM); transfecting at least one HLA class I positive cell with at least one tandem minigene construct comprising at least one sequence encoding the at least one neoantigen; identifying a complex comprising the at least one HLA molecule and the at least one neoantigen peptide produced by the at least one HLA class I positive cell; forming a vaccine comprising the at least one neoantigen; and administering the vaccine to the subject, wherein at least one tumor cell of the cancer comprises at least one polypeptide comprising at least one amino acid substitution.
2. A method in accordance with aspect 1, wherein the at least one neoantigen peptide consists of from 9 to 11 amino acids.
3. A method in accordance with aspect 1, wherein the at least one neoantigen peptide consists of 9 amino acids.
4. A method in accordance with aspect 1, wherein the at least one neoantigen binds in silico to an HLA class I molecule with an affinity of <250 nM.
5. A method in accordance with aspect 1, wherein the at least one neoantigen binds in vitro to an class I molecule with an affinity of <3.8 log (IC50, nM).
6. A method in accordance with aspect 1, wherein the at least one neoantigen binds in vitro to an HLA class I molecule with an affinity of <3.7 log (IC50, nM).
7. A method in accordance with aspect 1, wherein the at least one neoantigen binds in vitro to an HLA class I molecule with an affinity of <3.2 log (IC50, nM).
8. A method in accordance with aspect 1, wherein the vaccine comprises at least seven neoantigen peptides.
9. A method in accordance with aspect 1, wherein the HLA class I molecule is selected from the group consisting of HLA-A*01:01, HLA-B*07:02, HLA-A*02:01, HLA-B*07:03, HLA-A*02:02, HLA-B*08:01, HLA-A*02:03, HLA-B*15:01, HLA-A*02:05, HLA-B*15:02, HLA-A*02:06, HLA-B*15:03, HLA-A*02:07, HLA-B*15:08, HLA-*03:01, HLA-B*15:12, HLA-A*11:01, HLA-B*15:16, HLA-A*11:02, HLA-B*15:18, HLA-A*24:02, HLA-B*27:03, HLA-A*29:01, HLA-B*27:05, HLA-A*29:02, HLA-B*27:08, HLA-A34:02, HLA-B*35:01, HLA-A*36:01, HLA-B*35:08, HLA-B*42:01, HLA-B*53:01, HLA-B*54:01, HLA-B*56:01, HLA-B*56:02, HLA-B*57:01, HLA-B*57:02, HLA-B*57:03, HLA-B*58:01, HLA-B*67:01 and HLA-B*81:01.
10. A method in accordance with aspect 1, wherein the HLA class I molecule is an HLA-A*02:01 molecule.
11. A method in accordance with aspect 1, wherein the HLA class I molecule is an HLA-A*11:01 molecule.
12. A method in accordance with aspect 1, wherein the HLA class I molecule is an HLA-B*08:01 molecule.
13. A method in accordance with aspect 1, wherein the at least one HLA class I positive cell is at least one HLA class I positive melanoma cell.
14. A method in accordance with aspect 13, wherein the at least one HLA class I positive melanoma cell is selected from the group consisting of a DM6 cell and an A375 cell.
15. A method in accordance with aspect 1, wherein the tandem minigene further comprises a ubiquitination signal and two mini-gene controls.
16. A method in accordance with aspect 10, wherein the tandem minigene further comprises a ubiquitination signal and two mini-gene controls that encode HLA-A*02:01 peptides G280 and WNV SVG9.
17. A method in accordance with aspect 1, wherein the cancer is selected from the group consisting of skin cancer, lung cancer, bladder cancer, colorectal cancer, gastrointestinal cancer, esophageal cancer, gastric cancer, intestinal cancer, breast cancer, and a mismatch air deficiency cancer.
18. A method in accordance with aspect 17, wherein the skin cancer is selected from the group consisting of basal cell carcinoma, squamous cell carcinoma, merkel cell carcinoma, and melanoma.
19. A method in accordance with aspect 1, wherein the cancer is a melanoma.
20. A method in accordance with aspect 1, wherein the forming a vaccine comprises: providing a culture comprising dendritic cells obtained from the subject; and contacting the dendritic cells with the at least one neoantigen peptide, thereby forming dendritic cells comprising the at least one neoantigen peptide.
21. A method in accordance with aspect 20, further comprising: administering to the subject the dendritic cells comprising the at least one neoantigen peptide; obtaining a population of CD8+ T cells from a peripheral blood sample from the subject, wherein the CD8+ cells recognize the at least one neoantigen; and expanding the population of CD8+ T cells that recognizes the neoantigen.
22. A method in accordance with aspect 21, comprising administering to the subject the expanded population of CD8+ T cells.
23. A method in accordance with aspect 1, wherein the forming a vaccine comprises combining the neoantigen peptide with a pharmaceutically acceptable adjuvant.
24. A method in accordance with aspect 1, wherein the identifying a complex comprises a LC/MS assay.
25. A method in accordance with aspect 1, wherein the identifying a complex comprises a reverse phase HPLC assay.
26. A method of treating a cancer in a subject in need thereof, comprising: a) providing a sample of a tumor from a subject; b) performing exome sequencing on the sample to identify one or more amino acid substitutions comprised by the tumor exome; c) performing transcriptome sequencing on the sample to verify expression of the amino acid substitutions identified in b); and d) selecting at least one candidate neoantigen peptide sequence from amongst the amino acid substitutions identified in c) according to the following criteria; i) Exome VAF>10%; ii) Transcription VAF>10%; iii) Alternate reads>5; iv) FPKM>1; v) binds in silico to an HLA class I molecule with an affinity of <500 nM and a stability>2 h; e) performing an in vitro HLA class I binding assay; f) selecting at least one candidate neoantigen peptide sequence from amongst the amino acid substitutions identified in d) that bind HLA class one molecules with an affinity of <4.7 log (IC50, nM) in the assay performed in e); g) transfecting at least one HLA class I positive cell with at least one tandem minigene construct comprising at least one sequence encoding the at least one neoantigen; h) identifying a complex comprising the at least one HLA molecule and the at least one neoantigen peptide produced by the at least one HLA class I positive cell; i) forming a vaccine comprising the at least one neoantigen; and j) administering the vaccine to the subject, wherein, at least one tumor cell of the cancer comprises at least one polypeptide comprising the one or more amino acid substitutions.
27. A method in accordance with aspect 26, wherein the Exome VAF is ≧30%
28. A method in accordance with aspect 26, wherein the Exome VAF is ≧40%.
29. A method in accordance with aspect 26, wherein the Exome VAF is ≧50%.
30. A method in accordance with aspect 26, wherein the in vitro HLA class I binding assay is selected from the group consisting of a T2 assay and a fluorescence polarization assay.
31. A method in accordance with aspect 26, wherein the forming a vaccine comprises: providing a culture comprising dendritic cells obtained from the subject; and contacting the dendritic cells with the at least one neoantigen peptide, thereby forming dendritic cells comprising the at least one neoantigen peptide.
32. A method in accordance with aspect 31, further comprising; administering to the subject the dendritic cells comprising the at least one neoantigen peptide; obtaining a population of CD8+ T cells from a peripheral blood sample from the subject, wherein the CD8+ cells recognize the at least one neoantigen; and expanding the population of CD8+ T cells that recognizes the neoantigen.
33. A method in accordance with aspect 32, comprising administering to the subject cells of the expanded population of CD8+ T cells.
34. A method in accordance with aspect 26, wherein the forming a vaccine comprises combining the neoantigen peptide with a pharmaceutically acceptable adjuvant.
35. A method in accordance with aspect 26, wherein the identifying a complex comprising the at least one HLA molecule and the at least one neoantigen peptide comprises a LC/MS assay.
36. A method in accordance with aspect 26, wherein the identifying a complex comprising the at least one HLA molecule and the at least one neoantigen peptide comprises a reverse phase HPLC assay.
37. A method of treating a cancer in a subject in need thereof, comprising: providing a neoantigen peptide encoded in DNA of a tumor of the subject, wherein the neoantigen peptide consists of from 8 to 13 amino acids, binds in silico to an HLA class I molecule with an affinity of <500 nM and a stability>2 h, performing an in vitro HLA class I molecule binding assay to identify at least one neoantigen peptide which binds in vitro to an HLA class I molecule with an affinity of <4.7 log (IC50, nM); transfecting at least one HLA class positive cell with at least one tandem minigene construct comprising at least one sequence encoding the at least one neoantigen; identifying a complex comprising the at least one HLA molecule and the at least one neoantigen peptide produced by the at least one HLA class I positive cell; forming a vaccine comprising the at least one neoantigen; and administering the vaccine to the subject, wherein at least one tumor cell of the cancer comprises at least one polypeptide comprising at least one amino acid substitution.
38. A method in accordance with aspect 37, wherein the in vitro HLA class I binding assay is selected from the group consisting of a T2 assay and a fluorescence polarization assay.
39. A method in accordance with aspect 37, wherein the identifying a complex comprising the at least one HLA molecule and the at least one neoantigen peptide comprises a LC/MS assay.
40. A method in accordance with aspect 37, wherein the identifying a complex comprising the at least one HLA molecule and the at least one neoantigen peptide comprises a reverse phase HPLC assay.
41. A method in accordance with aspect 37, wherein the forming a vaccine comprises; providing a culture comprising dendritic cells obtained from the subject; and contacting the dendritic cells with the at least one neoantigen peptide, thereby forming dendritic cells comprising the at least one neoantigen peptide.
42. A method in accordance with aspect 41, further comprising; administering to the subject the dendritic cells comprising the at least one neoantigen peptide; obtaining a population of CD8+ T cells from a peripheral blood sample from the subject, wherein the CD8+ cells recognize the at least one neoantigen; and expanding the population of CD8+ T cells that recognizes the neoantigen.
43. A method in accordance with aspect 42, comprising administering to the subject the expanded population of CD8+ T cells.
44. A neoantigen peptide encoded in DNA of a tumor of the subject for use in the treatment of a cancer, wherein the neoantigen peptide consists of from 8 to 13 amino acids, binds in silico to an HLA class I molecule with an affinity of <500 nM and a stability>2 h and binds in vitro to an HLA class I molecule with an affinity of <4.7 log (IC50, nM), wherein the treatment comprises: transfecting at least one HLA class I positive cell with at least one tandem minigene construct comprising at least one sequence encoding the at least one neoantigen; identifying a complex comprising the at least one HLA molecule and the at least one neoantigen peptide produced by the at least one HLA class I positive cell; forming a vaccine comprising the at least one neoantigen; and administering the vaccine to the subject, wherein at least one tumor cell of the cancer comprises at least one polypeptide comprising at least one amino acid substitution.
45. A neoantigen peptide in accordance with aspect 44, wherein the forming a vaccine comprises: providing a culture comprising dendritic cells obtained from the subject; and contacting the dendritic cells with the at least one neoantigen peptide, thereby forming dendritic cells comprising the at least one neoantigen peptide.
46. A neoantigen peptide in accordance with aspect 45, wherein the treatment of a cancer further comprises: administering to the subject the dendritic cells comprising the at least one neoantigen peptide; obtaining a population of CD8+ T cells from a peripheral blood sample from the subject, wherein the CD8+ cells recognize the at least one neoantigen; expanding the population of CD8+ T cells that recognizes the neoantigen; and administering the expanded population of CD8+ cells to the subject.

### BRIEF DESCRIPTION OF THE DRAWINGS

FIG. 1 illustrates a work flow for identifying candidate neo-antigens and preparing a dendritic cell vaccine comprising the neo-antigens.

FIG. 2 illustrates the analytical steps and specific neo-antigen analysis for a melanoma patient.

FIG. 3 illustrates HLA binding on T-cell surfaces to various neo-antigens.

FIG. 4 illustrates a schematic representation of the steps for creating a dendritic cell based vaccine of the present teachings.

FIG. 5 illustrates T cell response in vaccinated patients for the listed neo-antigens using a dextramer assay.

FIG. 6 illustrates the in silico binding affinity (top) and stability (bottom) of peptides to T-cell HLA.

FIG. 7 illustrates the binding of immunogenic peptides to blood CD8 T cells following vaccination.

FIG. 8 illustrates antigen-specific T cell yields following vaccination.

FIG. 9 is a schematic diagram of a tandem mini-gene construct.

FIG. 10 illustrates ELISA-measured production of IFN-γ by T cells.

FIG. 11 illustrates that T cell specificity can detect a single amino acid change for AKAP13 and Sec24A.

FIG. 12 illustrates that T cells cannot discriminate between peptides with a single amino acid change for OR8B3.

FIG. 13 illustrates that vaccine-induced T cells produce large amounts of IFN-γ relative to IL-4, -5 and -13.

FIG. 14 illustrates tumor regression monitored by luciferase (photon flux).

FIG. 15 illustrates disease progression of mice inoculated with a luciferase expressing melanoma.

FIG. 16 illustrates the relationship between tumor regression and survival.

FIG. 17 illustrates immunological and clinical outcomes for patients treated with G209-2M and G2880-9V specific CD8+ T cells.

FIG. 18 illustrates ex-vivo IL-12 production and that Tc1 profile correlates with clinical outcome (TPP)

FIG. 19 illustrates that weak p35 transcription accounts for the IL-12p70 defect in non-responder patients.

FIG. 20 illustrates that impaired IL-12p70 production by a patient's dendritic cells is rescued by a combination of innate and adaptive signals.

FIG. 21 illustrates that a combination of innate and adaptive signals for dendritic cell maturation enhances the kinetics of the response.

FIG. 22 illustrates that a combination of innate and adaptive signals for dendritic cell maturation promotes Tc1 -polarized immunity.

FIG. 23 illustrates that cutaneous melanoma harbor a significant mutation burden.

FIG. 24 illustrates the translation of tumor missense mutations into patient-specific vaccines.

FIG. 25 illustrates discrimination between mutation and wild-type sequences and discrimination between antigens that are and are not presented to T-cells.

FIG. 26A-B illustrates clinical trial schema and ex-vivo IL-12p70 levels produced by mature DC.

FIG. 27 is a schematic representation of the selection of AAS peptides for use in experiments and vaccines.

FIG. 28 is a schematic representation of a strategy for neoantigen selection.

FIG. 29 illustrates AAS-comprising peptide binding to HLA-A*02:01.

FIG. 30A-C illustrate immune response to neoantigens.

FIG. 31 illustrates immune-monitoring of neoantigen-specific CD8+ T cell responses

FIG. 32 illustrates frequency of G209-2M- and G280-9V-specific T cells in CD8+ populations isolated directly from PBMC samples and after ex-vivo expansion using autologous DC and artificial antigen presenting cells.

FIG. 33 illustrates kinetics of immune responses to G209-2M and G280-9V peptides.

FIG. 34 illustrates antigenic determinants recognized by vaccine-induced T-cells

FIG. 35 illustrates cytokine production in neoantigen-specific T cells that were stimulated with artificial antigen presenting cells in the presence (open bar) or absence (close bar) of AAS-peptide.

FIG. 36 illustrates the Type 1/Type 2 phenotype of neoantigen-specific CD8+ T cells.

FIG. 37A-B illustrates the structure (A) and expression (B) of tandem mini-gene constructs (TMC) used for evaluating processing and presentation of neoantigens.

FIG. 38 illustrates neoantigen processing and presentation.

FIG. 39 illustrates interferon production in neoantigen-specific CD8 T cells cultured with neoantigen expressing DM6 cells.

FIG. 40A-H illustrates processing and presentation of tumor neoantigens.

FIG. 41A-D illustrates processing and presentation of to G280 and WNV SVG9 peptide controls.

FIG. 42 is a schematic diagram for analysis and identification of neoantigen-specific TCRβ clonotypes in CD8+ T cell populations isolated from PBMC samples obtained Pre- and Post-vaccination.

FIG. 43A-B illustrates profiles of purified neoantigen-specific CD8+ T cells used for the generation of TCRβ CDR3 reference libraries.

FIG. 44A-B illustrate that vaccination promotes a diverse neoantigen-specific T cell repertoire.

FIG. 45 depicts schematic diagrams of HLA-A*02:01 and HLA-B*08:01 neoantigen identification for patient MEL66.

FIG. 46 depicts schematic diagrams of HLA-A*02:01 and HLA-A*11:01 neoantigen identification for patient MEL69.

FIG. 47 depicts results of a dextramer assay to illustrate neoantigen response in T cells following administration of a vaccine in accordance with the present teachings.

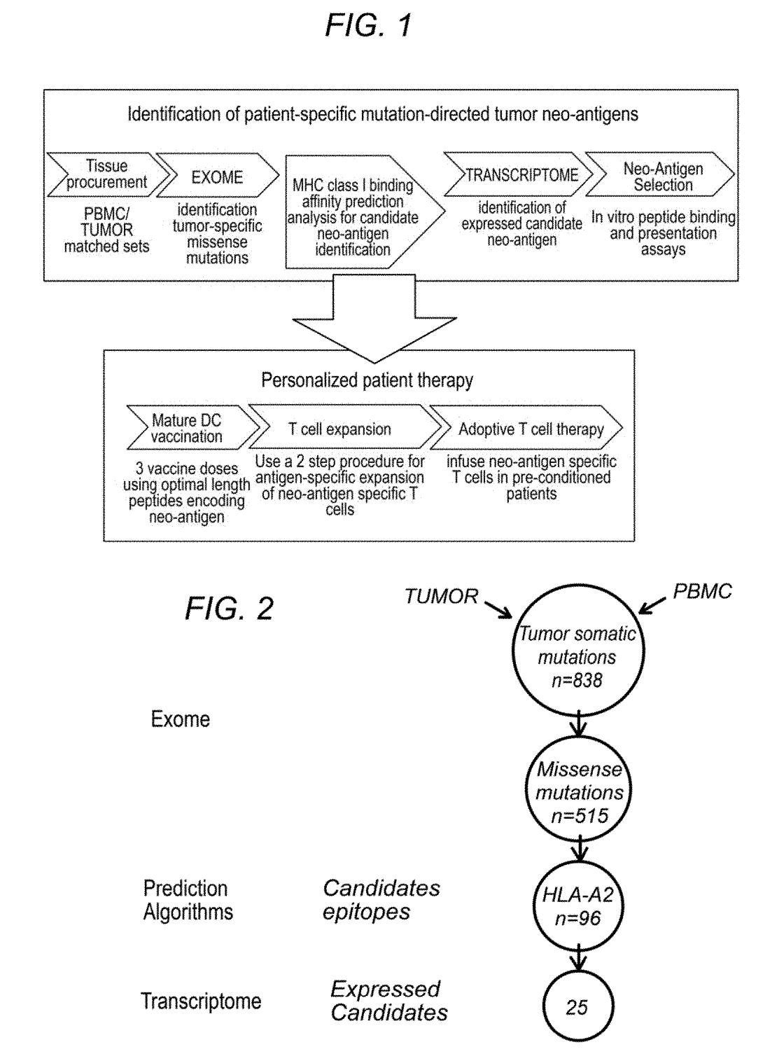

### DETAILED DESCRIPTION

The present teachings describe methods of creating vaccines for personalized cancer treatment. As used herein, “a vaccine” is a preparation that induces a T-cell mediated immune response. As used in the present description and the appended claims, the singular forms “a”, “an” and “the” are intended to include the plural forms as well, unless the context indicates otherwise.

In some embodiments, methods of the present teachings can comprise sequencing DNA from excised tumor tissue of a subject to identify amino acid substitutions, performing sequence capture to confirm the expression of the amino acid substitutions, selecting amino acid substitutions that bind or are likely to bind HLA molecules, transfecting nucleic acids encoding the selected amino acid substitutions into an HLA positive melanoma cell line, extracting HLA class I complexes from the transfected cells, identifying the sequence of neoantigens bound to the extracted HLA class one complexes, contacting dendritic cells obtained from the subject with the identified neoantigen peptides, thereby forming a dendritic cell vaccine, administering to the subject the dendritic cell vaccine, obtaining and enriching CD8+ T cells from the subject, and administering the enriched CD8+ T cells to the subject. In some embodiments, the neoantigen binding T cells can be used for adaptive T cell therapy. In some embodiments, a fluorescence polarization binding assay can be used to confirm the binding of neoantigen peptides to HLA molecules prior to selection for transfection.

In some configurations, the following criteria can be used to select the neoantigens for transfection into HLA class I positive cells; in the exome sequencing, the variant allele fraction of the neoantigen greater than 10%; in the transcript sequencing results the VAF greater than 10%, the alternate read counts greater than 5, and the FPKM greater than 1; the encoded peptides can be 9-11 amino acids in length; the predicted binding to any HLA class I allele can have following characteristics; the predicted MHC binding <250 nM (NetMHC3.4 algorithm), the predicted MHC stability>2 h (NetMHCStab, algorithm); the experimental MHC binding<3.2 log [IC₅₀, nM] in the fluorescence polarization binding assay. In some embodiments, a personalized immunotherapy of the present teachings can be used in conjunction with check point inhibitors, such as but without limitation ipiplimumab therapy. In some configurations, a cancer vaccine can be generated by contacting dendritic cells obtained from the patient with at least one neoantigen peptide of the present teachings. In some configurations, the dendritic cell vaccine can then be administered to the subject. In some configurations, CD8+ T cells be obtained from PBMC samples from the subject, and CD8+ T cells that recognize the at least one neoantigen are isolated using cell sorting. In various configurations, the cell sorting can comprise using an affinity column or affinity beads. In some configurations, sorted CD8 + T cells that recognize neoantigens can be expanded using methods as described herein. In some configurations, the expanded T cells can then be administered to the subject.

In various configurations, the present teachings include a series of analytical steps for identification of neo-antigens from somatic tumor missense mutations, as illustrated in FIG. 2. In various embodiments, DNA isolated from tumor and matched PBMC can be subjected to exome sequencing in order to identify tumor somatic missense mutations. For example, in melanoma and lung cancer high number of missense mutations (>200) can be identified per tumor genome. Prediction algorithms such as, without limitation, PePSSI (Bui, H. H., et al., Proteins 63, 43-52, 2006) can be used for the identification of candidate tumor neoantigen epitopes presented in the context of the patient's HLA class I molecules. In various configurations, analysis of tumor transcriptome data can used for the identification and selection, among predicted candidates, of those epitopes that are expressed by the tumor.

### Methods

The methods and compositions described herein utilize laboratory techniques well known to skilled artisans, and can be found in laboratory manuals such as Sambrook, J., et al., Molecular Cloning: A Laboratory Manual, 3rd ed. Cold Spring Harbor Laboratory Press, Cold Spring Harbor, N.Y., 2001; Methods In Molecular Biology, ed. Richard, Humana Press, NJ, 1995; Spector, D. L. et al, Cells: A Laboratory Manual, Cold Spring Harbor Laboratory Press, Cold Spring Harbor, N.Y., 1998; and Harlow, E., Using Antibodies: A Laboratory Manual, Cold Spring Harbor Laboratory Press, Cold Spring Harbor, N.Y., 1999. Methods also are as described herein and in publications such as Linette, G. P. et al., Clin. Cancer Res. 11, 7692-7699, 2005; Carreno, B. M. et al., J. Immunol. 188, 5839-5849, 2012; and Carreno, B. M., et al., J. Clin. Invest. 123, 3383-3394, 2013.

In order to determine the safety, tolerability and immunological responses to Amino Acid Substitutions (AAS)-peptides formulated in an mDC vaccine, the following protocols were followed.

### Human Subjects

### EXAMPLES 1-10

Human subjects, Eligible adult patients with newly diagnosed treatment naïve (ECOG performance status 0) stage IV cutaneous melanoma are enrolled in this clinical trial. All subjects are HLA-A*0201*, had gp100⁺ biopsy-proven (HMB45⁺, immunohistochemistry) melanoma metastases, have no evidence of autoimmune disorder, and are negative for HIV, HBV, and HCV. Leukapheresis was performed to obtain PBMCs from patients and healthy donors through the Barnes Jewish Hospital blood bank. For trial patients, leukapheresis is performed prior to treatment and after D3 and D6. Patients are not prescreened for IL-12p70 DC production prior to treatment. Prior to treatment, baseline imaging is performed by MRI scan of brain and CT scan of the chest/abdomen/pelvis with i.v. contrast.

### EXAMPLES 11-15

All patients were enrolled in clinical trial (NCT00683670, BB-IND 13590) and signed informed consents that had been approved by the Institutional Review Board of Washington University. All subjects were HLA-A*02:01*, had no evidence of autoimmune disorder and were negative for HIV, HBV, and HCV. Leukapheresis was performed, prior to treatment and after the 3rd mature dendritic cell (DC) vaccination, at Barnes Jewish Hospital blood bank (Saint Louis, Mo.). Patients were not prescreened for interleukin (IL)-12p70 DC production prior to treatment. Prior to treatment, baseline imaging was performed by MRI scan of brain and CT scan of the chest, abdomen and pelvis with i.v. contrast. Toxicities and adverse effects were graded according to the National Cancer Institute Common Toxicity Scale (version 3.0). Informed consent for genome sequencing was obtained for all patients on protocols approved by the Institutional Review Board of Washington University.

### Patient Information

Patient MEL21 was a 54-year-old man diagnosed with stage 3C cutaneous melanoma of the right lower extremity in 2010. The BRAF V600E mutation was detected. Surgery was performed to excise 2 cm inguinal lymph node and numerous in transit metastases. He developed recurrent in transit metastases and deep pelvic adenopathy in May 2012 and was given ipilimumab (3 mg/kg×4 doses) with stable disease until late 2013. Disease progression was noted with increasing 2 cm external iliac, 1.2 cm inguinal, and 7 mm retrocrural adenopathy. Three surgically resected melanoma lesions (inguinal lymph node Jan. 30, 2011, leg skin May 10, 2012, leg skin Jun. 6, 2013) and PBMC were submitted for genomic analysis in order to identity somatic missense mutations. The patient provided written'informed consent for the study and underwent apheresis, and received cyclophosphamide 4 days prior to administration of the first vaccine dose. He received a <total of three vaccine doses without side effect or toxicity. Re-staging CT showed stable disease and be remains in follow up 9 months later.

Patient MEL38 was a 47-year-old woman diagnosed with stage 3C cutaneous flank melanoma and underwent surgical resection of an axillary lymph node in 2012. The BRAF V600E mutation was detected. She developed recurrent disease in the skin and axilla that was surgically resected. A few months later, CT imaging confirmed metastatic disease in the right lung and axilla and she was given ipilimumab (3 mg/kg×4 doses) in May 2012 with complications of grade 2 autoimmune colitis requiring prednisone taper and later, grade 3 hypophysitis requiring replacement therapy with levothyroxine and hydrocortisone. Disease progression was noted 12 months later with new lung and skin metastases. Vemurafenib was administered for two months with no response in August 2013. Three surgically resected melanoma lesions (axilla lymph node Apr. 19, 2012, skin breast Feb. 14, 2013, skin abdominal wall Apr. 16, 2013) and PBMC were submitted for genomic analysis in order to identify somatic missense mutations. Further disease progression was evident with 3 lung nodules measuring 12 mm, 5 mm, and 5 mm in diameter. The patient provided written informed consent for the study and underwent apheresis, and received cyclophosphamide 4 days prior to the first vaccine dose. She received a total of three vaccine doses without side effect or toxicity. Re-staging CT showed 30% tumor reduction; however, the following CT examination 12 weeks later showed interval increase of tumor size back to baseline dimensions with no new sites of disease. The patient remains with stable disease for the past 8 months.

Patient MEL218 was a 52-year-old man diagnosed with stage 3C cutaneous melanoma on the left lower extremity in 2005. The BRAF mutation V600E mutation was detected when tested later on archived tumor. He underwent surgical resection and received adjuvant interferon for 6 months but had disease recurrence that was surgically resected on several occasions. In 2008, he developed disease progression with extensive in transit and subcutaneous metastases on the left leg with bulky inguinal nodal metastasis deemed unresectable. He received ipilimumab (10 mg/kg×14 doses) on clinical trial from 2008-2012 with complete response. One surgical specimen (inguinal lymph node Apr. 4, 2005) and PBMC were submitted for genomic analysis to identify somatic missense mutations. The patient provided written informed consent for the study and underwent apheresis, and received cyclophosphamide 4 days prior to the first vaccine dose. He received a total of three vaccine doses administered in the adjuvant setting without side effect or toxicity. Re-staging PET-CT imaging confirms no evidence of recurrent or metastatic disease. The patient remains in complete remission and continues in follow up.

Patient MEL69 was a 61-year-old man diagnosed with stage 3C cutaneous melanoma in 2012. Surgery was performed to excise the primary site and the axillary adenopathy. A total of 3 lymph nodes contained metastatic melanoma. The BRAF V600E mutation was detected. The patient received adjuvant Interferon for 5 months but this was discontinued after progression and development of metastatic disease. The patient was given vemurafenib for 10 months but progressed with new sites of disease. Dabrafenib and trametinib combination systemic therapy was administered for 7 additional months until progression. Several new sites of metastatic disease including a solitary brain lesion were resected. His subsequent course was complicated by malignant pericardial effusion and deep venous thrombosis. After appropriate treatment, he improved. Two surgically resected melanoma lesions (MEL69A2, limb and MEL69B2, scalp) and PBMC were submitted for genomic analysis in order to identify somatic missense mutations. The patient provided written informed consent, underwent apheresis, and then received cyclophosphamide 4 d prior to the first vaccine dose. He received a total of 2 vaccines doses without side effect or toxicity. Re-staging CT examination confirmed disease progression and the patient was removed from the study and enrolled in hospice care.

Patient MEL66 was a 43-year-old female diagnosed initially with stage 3B cutaneous melanoma in 2013. Surgery was performed to excise in transit metastases and the BRAF V600E mutation was detected. Subsequent imaging confirmed metastatic disease in the lung and retroperitoneal cavity deemed unresectable. She received several doses of ipilimumab and developed grade 3 autoimmune colitis treated with corticosteroids. After her recovery, disease progression was noted and combination therapy with dabrafenib/trametinib was begun. Disease progression was noted after 6 months of treatment. Surgical resection of several metastatic lesions was performed to render the patient disease-free. Two surgically resected melanoma lesions (ME1-66A, skin and. MEL66D, soft tissue) and PBMC were submitted for genomic analysis in order to identify somatic missense mutations. The patient provided written informed consent, underwent apheresis, and then received cyclophosphamide 4 d prior to the first vaccine dose. She received a total of 3 vaccine doses without side effect or toxicity. Re-staging Ct confirmed no evidence of disease recurrence and the patient remains in remission with no evidence of disease 4 months in follow up with no additional therapy.

### Cyclophosphamide Treatment and DC Preparation (Examples 1-10)

Cyclophosphamide (300 mg/m²) was given 72 hours prior to D1 with the intention of eliminating Tregs (Hoons, D. S., et al., Cancer Res., 50, 5358-5364, 1990). All mature dendritic cell (mDC) vaccine doses were prepared at the time of immunization from either freshly isolated (D1) or cryopreserved (D2-D6) PBMCs (all derived from the same leukapheresis collection). A GMP-grade CD40Lexpressing K562 cell line (referred to as K463H), used for maturation of DCs, is generated, selected, and maintained under serum-free (Stemline, S1694 media) conditions. For each vaccine dose, monocyte-derived immature dendridic cells (iDCs) were generated as described previously (Linette, G. P., et al., Clin. Cancer Res., 11, 7692-7699, 2005) by culturing the PBMC adherent fraction in RPMI 1640 with 1% human AB-serum (DC media) supplemented with 100 ng/ml GM-CSF (Berlex) and 20 ng/ml IL-4 (CellGenix). 6 days after culture initiation, iDCs were harvested, washed in PBS, and cultured for an additional 24 hours in DC media (iDC control) or DC media with irradiated (100 Gy) K463H (5:1 DC/K463H ratio) and 100 U/ml IFN-γ (Actimmune; InterMune Inc.) to generate mDCs. 2 hours prior to infusion, mDCs were pulsed with (50 μg/10⁶ cells/ml) peptide. For infusion, mDCs were resuspended in 50 ml normal saline supplemented with 5% human serum albumin and administered over 30 minutes by i.v. infusion after premedication with 650 mg acetaminophen.

### DC Immunizations (Examples 1-10)

mDC infusions were given i.v. every 3 weeks for 6 doses in the outpatient clinic. A restaging CT scan of the chest/abdomen/pelvis with i.v. contrast was performed after D3 and D6 and then every 2 months thereafter until disease progression. If clinical or radiographic disease progression was evident, the patient was removed from the study. For D1, patients received 1.5+10⁷ DCs per peptide (6×10⁷ DCs total); for D2-D6, patients received 5×10⁶ DCs per peptide (2×10⁷ DCs total). Patients underwent clinical evaluation prior to each mDC infusion. Toxicities and adverse effects were graded according to the National Cancer Institute Common Toxicity Scale (version 3.0). Clinical response was assessed by measurement of assessable metastatic deposits by CT scan, MRI scan, or direct measure of cutaneous deposits. The RECIST (v1.0) group system was used (Therasse, P., et al., J. Nat'l. Cancer Inst., 92, 205-216, 2000).

Immunologic monitoring (Examples 1-10). Immunologic analysis to evaluate the kinetics and magnitude of T cell response to gp100 peptides was performed using PBMCs collected weekly (prior to vaccination and until week 21. Fresh PBMCs obtained by Ficoll-Hypaque gradient centrifugation were adjusted to 2×10⁶ cells/ml in Stemline media (Sigma-Aldrich) containing 5% human AB-serum, and dispersed at 1 ml/well in 24-well plates. Cultures were set up for the gp100 peptides and the CMV pp65 peptide (positive peptide control). Cultures were pulsed with 40 μg/ml peptide and 50 U/ml IL-2 fed starting at 48 hours and every other day thereafter. On day 12 (peak of response; the inventors' unpublished observation), cultures were harvested, counted, and stained for flow cytometry analysis. To assess the antigen-specific T cell frequency, cells were stained with HLAA*0201/peptide tetramers (Beckman Coulter) for 30 minutes at room temperature, followed by addition of FITC-conjugated CD4, CD14, CD19, and CD56 and allophycocyanin-conjugated CD8 (Invitrogen) for 15 minutes at 4° C. Cells were washed and resuspended in FACS buffer, and 7AAD was added 5 minutes before analysis. Control CMV pp65-specific CD8+ T cells were detected in all CMV-seropositive patients before and after immunization. A negative HLA-A*0201/HIV gag peptide tetramer control was included. 25,000 events in the CD8+ gate were collected using a hierarchical gating strategy that included FSC/SSC and excluded 7AAD+ (dead) cells and CD4+CD14+CD19+CD56+ cells. Data were acquired and analyzed using Flow-Jo software.

### DC Manufacturing and Vaccine (Examples 11-15)

Cyclophosphamide (300 mg/m²) was given 96 h prior to the first DC dose with the intention of eliminating Tregs. All mature DC (mDC) vaccine doses were prepared at time of immunization from either freshly isolated (D1) or cryopreserved (D2-3) PBMC (all derived from same leukapheresis collection). For each vaccine, dose, monocyte-derived immature DCs were generated in 100 ng/mL granulocyte-macrophage colony-stimulating factor (GM-CSF, Berlex) and 20 ng/mL IL-4 (Miltenyi Biotec) as described (Carreno, B. M., et al., J. Clin. Invest. 123, 3383-3394, 2013; Linette G P, et al., Clin. Cancer Res 11, 7692-7699, 2005) by culturing the PBMC adherent fraction in RPMI 1640 with 1% human AB-serum (DC media) supplemented with 100 ng/ml GM-CSF (Berlex) and 20 ng/ml IL-4 (CellGenix). Six days after culture initiation, immature DCs were cultured with irradiated (10,000 rad) GMP-grade CD40L-expressing K562 cells (Carreno, B. M., et al., J. Clin. Invest., 123, 3383-3394, 2013), 100 u/mL IFN-γ (Actimmune, InterMune Inc.), poly I:C (Invivogen, Inc) and R848 (Invivogen, Inc.) for 16 h to generate mDC. Two hours prior to infusion, mDC were pulsed (50 μg/10⁶ cells/mL) separately with each peptide (7 AAS-peptides and 2 gp100 peptides, G209-2M and G280-9V) and, for dose 1 only, influenza virus vaccine (Fluvirin Novartis) was added to provide a source of recall antigen for CD4+ T cells. IL-12p70 production by vaccine DC was measured by ELISA (eBioscience) in accordance to the manufacturer's instructions. The initial priming dose was 1.5×10⁷ DC per peptide (1.35×10⁸ DC total), in remaining doses, patients received 5×10⁶ DC per peptide (4.5×10⁷ DC total). mDC were resuspended in 50 mL normal saline supplemented with 5% human serum albumin and administered over 30 min by intravenous infusion after premedication with acetaminophen 650 mg. Patients underwent clinical evaluation prior to each mDC infusion.

Cytokine Production

DC IL-12p70 and IL-12p40 production is measured by ELISA (eBioscience) according to the manufacturer's instructions. Production of additional cytokines and chemokines by DCs is determined using MILLIPLEX map Human Cytokine Panels I and II (EMD Millipore). For production of cytokines by T cells, G280-9V-specific T cells are expanded using mDCs and AT-SCT as described previously (infra and Carreno, B. M., et al., J. Immunol. 188, 5839-5849, 2012). The frequency of antigen-specific T cells after secondary stimulation is 2%-52%, as determined by HLA-A*0201/peptide tetramers (NIH tetramers Facility or Beckman Coulter). T cells are restimulated as described infra (Carreno, B. M., et al., J. Immunol. 188, 5839-5849, 2012), supernatants are collected at 24 hours, and production of cytokines is determined using MILLIPLEX® map Human Cytokine Panel I (EMD Millipore).

### Generation and Expansion of Ag-Specific T Cells

CD8+ T cells were isolated from PBMCs using a CD82 negative-selection kit (Miltenyi Biotec, Auburn, Calif.). Purified CD8+ T cells were cultured at a 20:1 ratio with irradiated (2500 rad) autologous mature DC (mDC) pulsed with peptide in Stemline media (S1694; Sigma-Aldrich, St. Louis, Mo.) supplemented with pooled human sera (Stemline-5), Human IL-2 (10-50 U/ml; Chiron, Emeryville, Calif.) was added every 2 d starting 48 h after culture initiation. Fourteen days after DC stimulation, T cell cultures were harvested, characterized for neo-antigen specific frequencies using HLA/peptide tetramers (see below), and restimulated with irradiated (10,000 rad) Single Chain Trimers (SCT; U.S. Pat. No. 8,518,697; U.S. Pat. No. 8,895,020; Carreno, B. M., et al., J. Immunol., 188, 5839-5849, 2012) or amino-terminal extended peptide MHC class I single-chain trimer (AT-SCT)-expressing K562 cells at a 1:1 ratio. Cultures were initiated in either six-well plates (10⁶ each T and SCT or AT-SCT) or T25 flask (5×10⁶ each) using Stemline-5. Twenty-four hours after stimulation, cultures were supplemented with IL-2 (500 U/ml), and viable cell counts were performed daily.

Cell concentrations were maintained at 5×10⁵/ml throughout the culture period. For large-scale expansion, T cells were cultured in gas-permeable Lifecell bags (Nexell Therapeutics, Emeryville, Calif.). On days 10-14 of secondary stimulation, the percentage of tetramer+ cells and the number of viable cells were used to determine tetramer yields and tetramer folds.

For analysis of cytokines secreted by T cells upon SCT activation, cultures were activated 14 d after SCT or AT-SCT stimulation, T cells were restimulated with SCT at 1:1 ratio in RPMI 1640 supplemented with 5% pooled human sera (RPMI-5), supernatants were collected 24 h after activation and characterized using a MILLIPLEX® cytokine kit (Millipore, Billerica, Mass.), per the manufacturer's instructions.

qRT-PCR

qRT-PCR was performed as described previously (Carreno, B. M., et al., Immunol. Cell Biol. 87: 167-177, 2009). cDNAs were prepared (2 μg total RNA), and cDNA samples were amplified in triplicate using a GeneAmp 5700 sequencer detector (Applied Biosystems). Primers used are IL-12p35 (Hs00168405_m1) and ITGAX (integrin alpha X, referred to herein as CD11c; Hs01015070_m1). Transcript levels were calculated using the relative standard curve method, using CD11c transcript levels to normalize values.

### ⁵¹Cr Release and T2 Assays

⁵¹Cr release assays to measure specific lysis have been described previously (Carreno, B. M., et al., Immunol. Cell Biol., 87: 167-177, 2009; Linette, G. P. et al., Clin. Cancer Res. 11, 7692-7699, 2005). Melanoma cell lines DM6 (HLAA2+ gp100+) and A375 (HLA-A2+gp100−) were labeled with 25 μCi ⁵¹Cr for 1 hour, washed, and tested as targets in a standard 4-hour assay. Effectors were generated using PBMCs collected after D3 and cultured for 12 days in the presence of peptide (40 μg/ml) and IL2 (50 U/ml every other day). Vaccine-induced antigen-specific T cells were characterized using HLAA*0201/peptide dextramers (Immudex). To determine the avidity (effective concentration at 50% maximal lysis) of vaccine-induced T cells for antigen, T2 cells were pulsed with titrated G209-2M or G280-9V peptide concentrations for 1 hour in serum-free media followed by 51Cr (25 μCi) labeling for 1 hour, washed twice, and tested using vaccine-induced gp100-specific T cells in a standard 4-hour assay.

### Statistics

Student's t tests are 2-tailed (GraphPad Prism software, version 5.0). Data are presented as mean±1 SD, unless otherwise indicated. Cox regression analysis followed by likelihood-ratio test is used to evaluate whether (loge) IL-12p70 (sum) production added statistically significant information to a model of time to progression (TTP). Kaplan-Meier TTP model is used to test whether cytokine ratios added statistically significant information to a model of TTP. Wilcoxon matched-pairs analysis is used to compare IL-12p70 production between patients and healthy donors (GraphPad Prism software, version 5.0). All P values less than 0.05 were considered significant, except the Cox proportional hazard model, which used a lower threshold of significance (P<0.048) to adjust for 1 interim analysis of this endpoint.

### Peptides

Peptides were obtained lyophilized from American Peptide Company (>95% purity), dissolved in 10% DMSO in sterile water and tested for sterility, purity, endotoxin and residual organics. Peptide binding to HLA-A*02:01 was determined by T2 assay (Elvin et al. 1993 J. Immunol. Methods 158, 161) or using a fluorescence polarization assay (Pure Protein, L.L.C.) (Buchli, R., et al., Biochemistry 44, 12491-12507, 2005). The affinity scale of this latter assay is: high binders: log (IC₅₀ nM)<3.7; intermediate binders: log (IC₅₀ nM) 3.7-4.7; low binders: log (IC₅₀ nM) 4.7-5.5; and very low binders: log (IC₅₀ nM)≧6.0 (11).

### Computer Algorithm

Burrows-Wheeler Aligner (BWA; Li, H. and Durbin R., Bioinformatics 25, 1754-1760, 2009) is a reference-directed aligner that is used for mapping low-divergent sequences against a large reference genome, and consists of separate algorithms designed for handling short query sequences up to 100 bp, as well as longer sequences ranged from 70 bp to 1 Mbp.

Picard (Broad Institute, Cambridge, Mass.) is a set of Java-based command-line tools for processing and analyzing high-throughput sequencing data in both Sequence Alignment/Map (SAM) text format and SAM binary (BAM) format. The ‘MarkDuplicates’ utility within Picard examines aligned records in the supplied SAM or BAM file to locate duplicate molecule and can be used to flag and/or remove the duplicate records.

SAMtools (Li, H., et al., Bioinformatics, 25, 2078-2079, 2009) is a suite of programs for interacting with and post-processing alignments in the SAM/BAM format to perform a variety of functions like variant calling and alignment viewing as well as sorting, indexing, data extraction and format conversion.

Somatic Sniper (Larson, D. E., et al., Bioinformatics, 28, 311-317) is used to identify single nucleotide positions that are different between tumor and normal BAM files. It employs a Bayesian comparison of the genotype likelihoods in the tumor and normal, as determined by the germline genotyping algorithm implemented in the MAQ and then calculates the probability that the tumor and normal genotypes are different.

VarScan (Koboldt D. C., et al., Genome Research, 22, 568-576, 2012; Koboldt, D. C., et al., Bioinformatics 25, 2283-2285, 2009,) is a software program that detects somatic variants (SNPs and indels) using a heuristic method and a statistical test based on the number of aligned reads supporting each allele using an input SAMtools pileup/mpileup file. For tumor-normal pairs, it further classifies each variant as Germline, Somatic, or LOH, and also detects somatic copy number changes.

Strelka (Saunders, C. T., et al., Bioinformatics 28, 1811-1817, 2012) is an analysis package designed to detect SNVs and small indels from the sequencing data of matched tumor-normal samples. It is specifically designed to detect somatic variants at lower frequencies typically encountered in tumors due to high sample impurity or sub-clone variation, while maintaining sensitivity.

TopHat (Trapnell. C., et al., Bioinformatics, 25, 1105-1111, 2009; Kim, D., et al., Genome Biol., 14, R36, 2013) is a fast splice junction mapper for RNA-Seq reads that aligns reads to mammalian-sized genomes in order to identify exon-exon splice junctions. It uses the ultra high-throughput short read aligner Bowtie, and then analyzes the mapping results to identify splice junctions between exons.

Cufflinks (Trapnell, C., et al., Nat. Protoc., 7, 562-578, 2012) is a software program for transcriptome assembly and differential expression analysis for RNA-Seq data. It assembles transcripts from aligned RNA-Seq reads, estimates their abundances based on how many reads support each one, taking into account biases in library preparation protocols, and then tests for differential expression and regulation in RNA-Seq samples.

Flexbar (Dodt, M., et al., Biology (Basel), 1, 895-905, 2012) is a software package that preprocesses high-throughput sequencing data efficiently by demultiplexing barcoded runs and removing adapter sequences. Additionally, it supports trimming as well as filtering features; thereby aiming to increase read mapping rates and improve genome and transcriptome assemblies.

NetMHC 3.4 server (Nielsen, M., et al., Protein Sci., 12, 1007-1017, 2003; Lundegaard, C., et al., Nucleic Acids Res., 1, W509-512, 2008) makes high-accuracy predictions of major histocompatibility complex (MHC): peptide binding to a number of different HLA alleles. The predictions are based on artificial neural networks trained on different datasets (human and non-human) from several MHC alleles and position-specific scoring matrices (PSSMs).

In terms of additional filtering of variants from DNA/RNA data that would pass to analysis for identifying peptides, the following filters were used on coverage for tumor and normal, below which a variant is discarded from further consideration:

>=5× Normal coverage

>=10× Tumor coverage

<=2% Normal VAF

>=30% Tumor VAF

FPKM>1 (this is the only RNA-based filter).

In silico work flow.

The present inventors have developed an in silico automated pipeline for neoantigen prediction (pVAC-Seq) that can utilize several types of data input from next-generation sequencing assays. First a list of nonsynonymous mutations is identified by a somatic variant-calling pipeline using exomic sequencing and transcript sequencing of both normal and tumor tissue. This variant list can then be annotated with amino acid changes and transcript sequence. The HLA-haplotypes of the patient, can be derived through clinical genotyping assays or in silico approaches. These data can be input into the pVAC-Seq workflow which implements three steps: performing, epitope prediction, integrating sequencing-based information and lastly, filtering neoantigen candidates. The following paragraphs describe the analysis methodology from preparation of inputs to the selection of neoantigen vaccine candidates via pVAC-Seq.

Prepare Input Data: HLA-Typing, Alignment, Variant Detection and Annotation

As described above, pVAC-Seq utilizes input data generated from the analysis of next-generation sequence data that includes annotated nonsynonymous somatic variants that have been translated into mutant amino acid changes, as well as patient-specific HLA haplotypes. While these data could be obtained from any appropriate variant calling, annotation and HLA typing pipeline, the inventors' approach as disclosed herein utilized the following analysis methods for preparing these input data. In brief, BWA (version 0.5.9) (Li, H. and Durbin, R., Bioinformatics, 25, 1754-1760, 2009) was used as the aligner of choice with default parameters except the number of threads was set to 4 (−t 4) for faster processing, and the quality threshold for read trimming to 5 (−q 5). The resulting alignments were de-duplicated via Picard MarkDuplicates (version 1.46; Broad Institute, Cambridge, Mass.).

In cases where clinically genotyped HLA haplotyping calls were not available, the inventors used in silico HLA typing by HLAminer (Version1)(Warren, R. L., et al., Genome Med., 4, 95, 2012) to provide HLA haplotypes from either whole genome sequence data or RNA-seq data, or by Athlates (Liu, C., et al., Nucleic Acids Res, 41, e142, 2013) when exome data were available. Typing was performed on samples of the patient's normal cells, rather than cells from the tumor sample. The two software tools were >85% concordant in the inventors' test data; both algorithms were used in order to break ties reported by HLAminer (see below).
1. HLAminer for in silico HLA-typing using WGS data: When predicting HLA class I alleles tram WGS data, the inventors used HLAminer in de novo sequence alignment mode using TASR (Warren, R. L. and Holt, R. A., PLoS One., 6, e19816, 2011) (params: −i 1 −m 20) by running the script HPTASRwgs_classI.sh, provided in the download. (The download includes detailed instructions for customizing this script, and the scripts on which it depends, for the user's computing environment.) For each of the three HLA loci, HLAminer reports predictions ranked in decreasing order by score, where “Prediction #1” and “Prediction #2” are the most likely alleles for a given locus. When ties were present for Prediction 1 or Prediction 2, the inventors used all tied predictions downstream neo-epitope prediction. However, it should be noted that most epitope prediction algorithms, including NetMHC (Lundegaard, C., et al., Nucleic Acids Res., 36, 509-512, 2008; Nielsen, M., et al., Protein Sci., 12, 1007-1017, 2003), only work with an algorithm-specific subset of HLA alleles, so we are constrained to the set of NetMHC-compatible alleles. The current version NetMHC v3.4 supports 78 human alleles.
II. Athlates for in silico HLA-typing using exome sequence data: The inventors diverged from the recommended procedure to run Athlates at two points in the procedure: 1) they performed the alignment step to align exome sequence data (corresponding to the normal tissue sample) against the HLA allele sequences present in the IMGT/HLA database (Robinson, J., et. al., Nucleic Acids Res., 41, D1222-D1227, 2013), using BWA with zero mismatches (params: bwa aln −e 0 −o 0 −n 0) instead of NovoAlign (Hercus. C., Novocraft short read alignment package, 2009) with one mismatch, and 2) in the subsequent step, sequence reads that matched, for example, any HLA-A sequence from the database were extracted from the alignment using bedtools (Quinlan, A. R. and Hall, I. M., Bioinformatics 26, 841-842, 2010) instead of Picard. This procedure is resource-intensive, and may require careful resource management. Athlates reports alleles that have a Hamming distance of at most 2 and meet several coverage requirements. Additionally, it reports “inferred allelic pairs,” which are identified by comparing each possible allelic pair to a longer list of candidate alleles using a Hamming distance-based score. The inventors typically used the inferred allelic pair as input to subsequent steps in the neo-epitope prediction pipeline.

After alignments (and optional HLA typing) were completed, somatic mutation detection was performed using the following series of steps. (1) Samtools (Li, H., et al., Bioinformatics, 25, 2078-2079, 2009; Li, H. Bioinformatics, 27, 2987-2993, 2011) mpileup v0.1.16 was run with parameters ‘−A −B’ with default setting for the other parameters. These calls were filtered based on GMS ‘snp-filter v1’ and were retained if they met all of the following rules: (a) Site is greater than 10 bp from a predicted indel of quality 50 or greater, (b) The maximum mapping quality at the site is ≧40, (c) Fewer than 3 SNV calls are present in a 10 bp window around the site, (d) The site is covered by at least 3 reads and less than 1×109 reads, and (e) Consensus and SNP quality is ≧20. The filtered Samtools variant calls were intersected with those from Somatic Sniper version 1.0.2 (Larson, D. E., et al., Bioinformatics, 28, 311-317, 2012) (params: −F vcf q 1 −Q 15), and were further processed through the GMS ‘false-positive filter v1’ (params: -bam-readcount-version 0.4-bamreadcount-min-base-quality 15-min-mapping-quality 40-min-somatic-score 40). This filter used the following criteria for retaining variants: (a) ≧1% of variant allele support comes from reads sequenced on each strand, (b) variants have ≧5% Variant Allele Fraction (VAF) (c) more than 4 reads support the variant, (d) the average relative distance of the variant from the start/end of reads is greater than 0.1, (e) the difference in mismatch quality sum between variant and reference reads is less than 50, (f) the difference in mapping quality between variant and reference reads is less than 30, (g) the difference in average supporting read length between variant and reference reads is less than 25, (h) the average relative distance to the effective 3′ end of variant supporting reads is at least 0.2, and (i) the variant is not adjacent to 5 or more bases of the same nucleotide identity (e.g. a homopolymer run of the same base), (2) VarScan Somatic version 2.2.6 (Koboldt, D. C., et al., Bioinformatics, 25, 2283-2285, 2009; Koboldt, D. C., et al., Genome Res., 22, 568-576, 2012) was run with default parameters and the variant calls were filtered by GMS filter ‘varscan-high-confidence filter version v1’. The ‘varscan-high-confidence v1’ filter employed the following rules to filter out variants (a) p-value (reported by Varscan) is greater than 0.07, (5) Normal VAF is greater than 5%, (c) Tumor VAF is less than 10% or (d) less than 2 reads support the variant. The remaining variant calls were then processed through false-positive filter v1 (params: -bam-readcount-version 0.4-bamreadcount-min-base-quality 15) as described above. (3) Strelka version 1.0.10 (Saunders, C. T., et al., Bioinformatics, 28, 1811-1817, 2012) (params: isSkipDepthFilters=1).

The consolidated list of somatic mutations identified from these different variant-callers was then annotated using our internal annotator as part of the GMS pipeline. This annotator leverages the functionality of the Ensembl database (Flicek, P, et al., Nucleic Acids Res., 41, D48-55, 2013) and Variant Effect Predictor (VEP)(McLaren, W., et al., Bioinformatics, 26, 2069-2070, 2010).

From the annotated variants, there are two components that are needed for pVAC-Seq: amino acid change and transcript sequence. Even a single amino acid change in the transcript arising from missense mutations can alter the binding affinity of the resulting peptide with the MHC Class I molecule. Larger insertions and deletions, such as, for example, those arising from frameshift and truncating mutations, splicing aberrations or gene fusions can also result in potential neoantigens. However, for the present iterations of pVAC-Seq, the inventors chose to focus their analysis on only missense mutations.

One feature of the inventor's pipeline is the ability to compare the differences between tumor neo-antigens and normal peptides in terms of the peptide binding affinity. Additionally, it leverages RNA-Seq data to incorporate isoform-level expression information and to quickly cull variants that are not expressed in the tumor. To integrate RNA-Seq data, both transcript ID as well as the entire wild-type transcript amino acid sequence can be used as part of the annotated variant file.

### Perform Epitope Prediction

One component of pVAC-Seq is predicting epitopes that result from mutations by calculating their binding affinity against the Class I MHC molecule. This process involves the following steps for effectively preparing the input data as well as parsing the output.

### Generate FASTA File of Peptide Sequences:

Peptide sequences are an input to the MHC binding prediction tool, and the existing process to compare the germline normal with the tumor can be very onerous. To streamline the comparison, the inventors first build a FASTA file that consists of two amino acid sequences per variant site—wild-type (normal) and mutant (tumor). The FASTA sequence can be built using approximately 8-10 flanking amino acids on each side of the mutated amino acid. However, if the mutation is towards the end or beginning of the transcript, then the preceding or succeeding 16-20 amino acids can be taken respectively, as needed, to build the FASTA sequence. Subsequently, a key file can be created with the header (name and type of variant) and order of each FASTA sequence in the file. This can be done to correlate the output with the name of the variant protein, as subsequent epitope prediction software strips off each name.

### Run Epitope Prediction Software:

To predict high affinity peptides that bind to the HLA class I molecule, the standalone version of NetMHC 3.4 is used. The input to this software is the HLA type of the patient, determined via genotyping or using in silico methods, as well as the FASTA file generated in the previous step comprised of mutated and wild-type 17-21-mer sequences. Typically, antigenic epitopes presented by MHC class I molecules can vary in length from 8 to 13 or 8 to 11 amino acids. Therefore, specifying the same range when running epitope prediction software is recommended.

### Parse and Filter the Output:

Starting with the output list of all possible epitopes from the epitope prediction software, the inventors apply specific filters to choose the best mutant peptide incorporating candidates. First, further consideration is restricted to strong to intermediate binding peptides by focusing on candidates with a mutant (MT) binding score of less than 500 nM or less than 250 nM. Second, epitope binding calls are evaluated only for those peptides that contain the mutant amino acid (localized peptides). This filter eliminates any wild-type (WT) peptides that may overlap between the two FASTA sequences. The pVAC-seq workflow enables screening across multiple lengths and multiple alleles very efficiently. If predictions are run to assess multiple epitope lengths (e.g., 9-mer, 10-mer, etc.), and/or to evaluate all different patient HLA allele types, the inventors review all localized peptides and choose the single best binding value representative across lengths (9aa, 10aa, etc.) based on lowest binding score for MT sequence. Furthermore, they choose the ‘best candidate’ (lowest MT binding score) per mutation between all independent HLA allele types that were used as input.

### Integrate Expression and Coverage Information

Subsequently several filters are applied to ensure that the predicted neoantigens are expressed as RNA variants, and are predicted correctly based on coverage depth in the normal and tumor tissue data sets. Specifically, gene expression levels from RNA-Seq data measured as Fragments per kilobase of exon per million reads mapped (FPKM) provide a method to filter only the expressed transcripts. We used the tuxedo suite—Tophat (Trapnell, C. et al., Bioinformatics, 25, 1105-1111, 2009; Kim, D., et al., Genome Biol., 14, R36, 2013) and Cufflinks (Trapnell, C., et al., Nat. Protoc., 7, 562-578, 2012) as part of the GMS to align RNA-Seq data and subsequently infer gene expression for our in-house sequencing data. Depending on the type of RNA prep kit, OVATION® RNA-Seq System V2 (NuGEN Technologies, Inc. San Carlos, Calif.) or TRUSEQ® Stranded Total RNA Sample Prep kit (ILLUMINA®, Inc. San Diego, Calif.), used, Tophat was run with the following parameters: Tophat v2.0.8 ‘-bowtie-version-2.1.0’ for OVATION®, and ‘-library-type fr-firststrand-bowtie-version=2.1.0’ for TRUSEQ®. For OVATION® data, prior to alignment, paired 2×100 bp sequence reads were trimmed with Flexbar version 2.21 (Dodt, M., et al. Biology (Basel), 1, 895-905, 2012.) (params: -adapter CTTTGTGTTTGA (SEQ. ID NO: 474)-adapter-trim-end LEFT-nono-length-dist-threads 4-adapter-min-overlap 7-maxuncalled 150-min-readlength 25) to remove single primer isothermal amplification adapter sequences. Expression levels (FPKM) were calculated with Cufflinks v2.0.2 (params-max-bundle-length=10000000-num-threads 4).

For selecting unique vaccine candidates, targeting the best ‘quality’ of mutations is an important factor for prioritizing peptides. Sequencing depth as well as the fraction of reads containing the variant allele (VAF) are used as criteria to filter or prioritize mutations. This information was added in our pipeline via bam-readcount (Larson, D., The Gnome Institute at Washington University). Both tumor (from DNA as well as RNA) and normal coverage are calculated along with the VAF from corresponding DNA and RNA-Seq alignments.

### Filter Neoepitope Candidates

Since manufacturing antigenic peptides can be one of the most expensive steps in vaccine development and efficacy depends on selection of the best neoantigens, the inventors filter the list of predicted high binding peptides to the most highly confident set, primarily with expression and coverage based filters.

### The Filters can be Employed as Follows:

Depth based filters: any variants with normal coverage <=5× and normal VAF of >=2% can be filtered out. The normal coverage cutoff can be increased up to 20× to eliminate occasional misclassification of germline variants as somatic. Similarly, the normal VAF cutoff can be increased based on suspected level of contamination by tumor cells in the normal sample. For tumor coverage from DNA and/or RNA, a cutoff can be placed at >=10× with a VAF of >=10% or 30%. This can ensure that neoantigens from the major clones in the tumor are included, but the tumor VAF can be lowered to capture more variants, which may or may not be present in all tumor cells. Alternatively, if the patients are selected based on a pre-existing disease-associated mutation such as BRAF V600E in the case of melanoma, the VAF of the specific presumed driver mutation can be used as a guide for assessing clonality of other mutations.

Expression based filters: as a standard, genes with FPKM values of greater than zero are considered to be expressed. The inventors slightly increase this threshold to 1, to eliminate noise. Alternatively, the FPKM distribution (and the corresponding standard deviation) can be analyzed over the entire sample, to determine the sample-specific cutoffs for gene expression. Spike-in controls can also be added to the RNA-Seq experiment to assess quality of the sequencing library and to normalize gene expression data. This filtered list of mutations can be manually reviewed via visual inspection of aligned reads in a genome viewer like IGV (Robinson, J. T., et al., Nat Biotechnol., 29, 24-26, 2011; Thorvaldsdottir, H., et al. Brief Bioinform., 14, 178-192, 2013) to reduce the retention of obvious false positive mutations.

### Analysis of T Cell Responses

For functional characterization, neoantigen-specific T cell lines were generated using autologous mDC and antigen loaded artificial antigen presenting cells at a ratio of 1:1 as previously described (Carreno, B. M., et al., J. Immunol., 188, 5839-5849, 2012). To determine the peptide avidity (effective concentration at 50% maximal lysis, EC50) of neoantigen-specific T cells, T2 cells were pulsed with titrated peptide concentrations for 1 h, followed by ⁵¹Cr (25μCi) labeling for 1 h, washed twice and tested in a standard 4 h ⁵¹Cr release assay using neoantigen-specific cells as effectors. For production of cytokines, neoantigen-specific T cells were restimulated using artificial antigen presenting cells in the presence or absence of peptide, supernatants collected at 24 h and cytokine produced determined using MILLIPLEX® MAP Human Cytokine Panel I (EMD Millipore).

### Overview of the Present Teachings

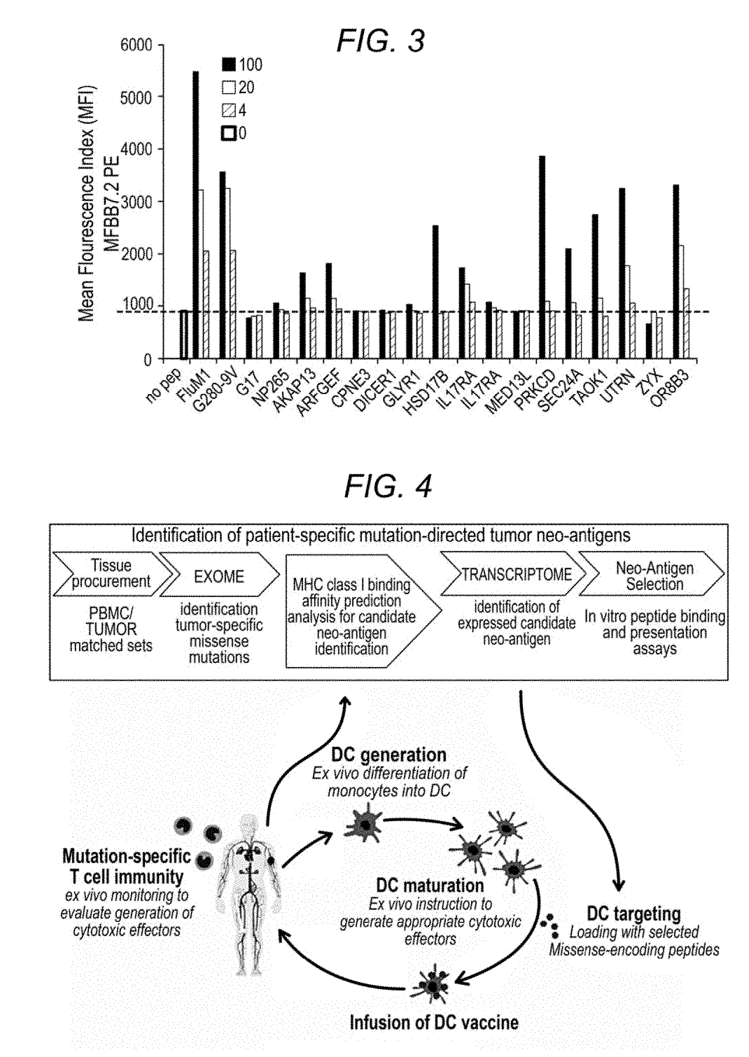

FIG. 4 illustrates a scheme showing neo-antigen identification and its incorporation into a personalized dendritic cells vaccine. The upper diagram depicts a pipeline for neoantigen identification. Tumor cells and matched peripheral blood mononuclear cells (PBMC) are subjected to whole exome sequencing to identify somatic missense mutations. Missense mutations are evaluated as peptides (8-13 aa long) through MHC class I binding and algorithms to identify potential candidate neoantigens and the expression of transcripts encoding mutated protein is confirmed by transcriptome sequencing. Synthetic peptides encoding candidate neoantigens can be tested experimentally for MHC class I binding and vaccine candidates can be selected using characteristics described infra. The lower diagram represents a vaccination process whereby dendritic cells (DC) can be generated from monocytes using GM-CSF and IL-4, and matured using CD40L/IFN-g/poly IC and R848. Mature DC can be pulsed with candidate neoantigen peptides and infused in order to generate mutation (missense)-specific T cells.

### EXAMPLES

The present teachings it descriptions that are not intended to limit the scope of any aspect or claim. Unless specifically presented in the past tense, an example can be a prophetic or an actual example. The examples and methods are provided to further illustrate the present teachings. Those of skill in the art, in light of the present disclosure, will appreciate that many changes can be made in the specific embodiments that are disclosed and still obtain a like or similar result without departing from the spirit and scope of the present teachings.

### Example 1

This example illustrates the clinical use of common cancer antigen peptides and the difficulties of using matured dendritic cells in cancer vaccines.

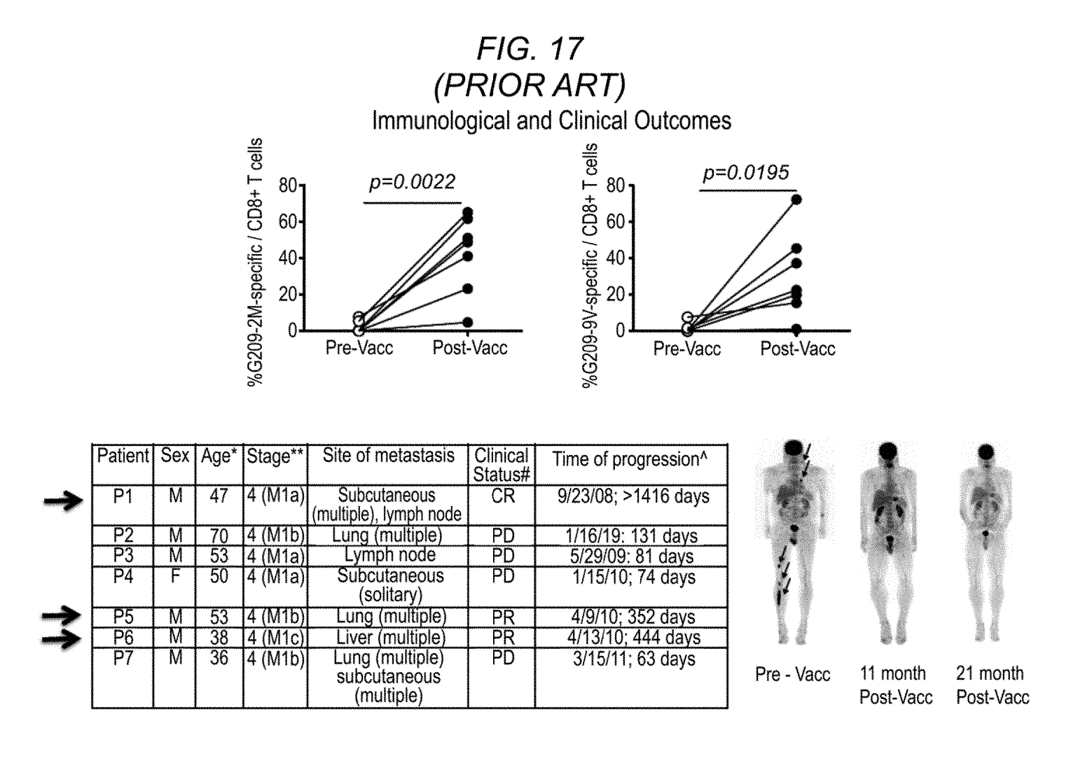

Vaccination was performed with HLA-A*0201-restricted gp100 melanoma antigen-derived peptides (G209-2M, and G280-9V) (Carreno, B. M., et al., J. Clin. Investigation, 123, 3383-3394, 2013; Kawakami, Y., et al., J. Immunol., 154, 3961-3968, 1995; Skipper, J. C., et al., Int. J. Cancer, 82, 669-677, 1999) using autologous peptide-pulsed, CD40L/IFN-γ-activated mature DCs (mDCs). The top of FIG. 17 illustrates the comparison of gp100 (G209-2M and G280-9V)-specific T cell frequencies observed pre- and post-vaccine. Statistical assessment was performed using paired two-tail t-test; p values are indicated in figure. The table on FIG. 17 bottom left summarizes the characteristics of patients enrolled in the trial and details their clinical outcomes: CR, complete response; PR, partial response: PD, progressive disease.

The bottom left of FIG. 17 illustrates radiologic studies (FDG-PET/CT imaging) that were obtained on Patient 1 before vaccination, 11 months and 21 months after treatment. Coronal whole body PET images show complete regression of left supra-clavicular and hilar lymph nodes as well as multiple subcutaneous lesions on the right leg. P1 remains in remission as of December 2012.

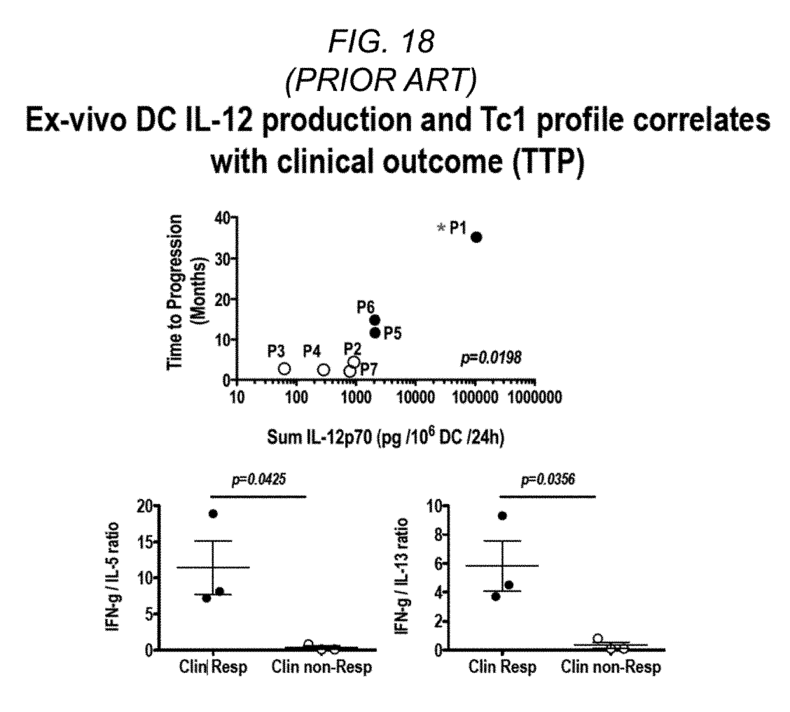

FIG. 18 illustrates that ex-vivo dendritic cell (DC) IL-12 production and Tc1 profile correlates with clinical outcome (TTP, time to progression) (Carreno, B. M., et al., J. Clin. Invest., 123, 3383-3394, 2013). A Cox regression analysis followed by likelihood-ratio test revealed a positive correlation between IL-12 production and TTP (FIG. 18, top; p=0.0198, log rank). Filled (dark) circles indicate patients that had a confirmed clinical response (P1, CR; P5 and P6, PR; FIG. 17, bottom left) with disease progression observed at or after 11.5 months of treatment initiation. The open (white) circles represent patients with rapid disease progression. The analysis was performed on Aug. 5, 2012, P1 remains in complete remission 4 years after initiation of treatment. No correlation was observed between IL-12 production and immune response or immune response and clinical outcome. Cytokine ratios differed among clinical responders (Clin Resp) and non-responder (Clin non-Resp) patients and demonstrate a Tc1 profile (FIG. 18, bottom; high IFN-g, low IL-5 or IL-13) among responders. p values are indicated in figure, unpaired two-tailed t-test.

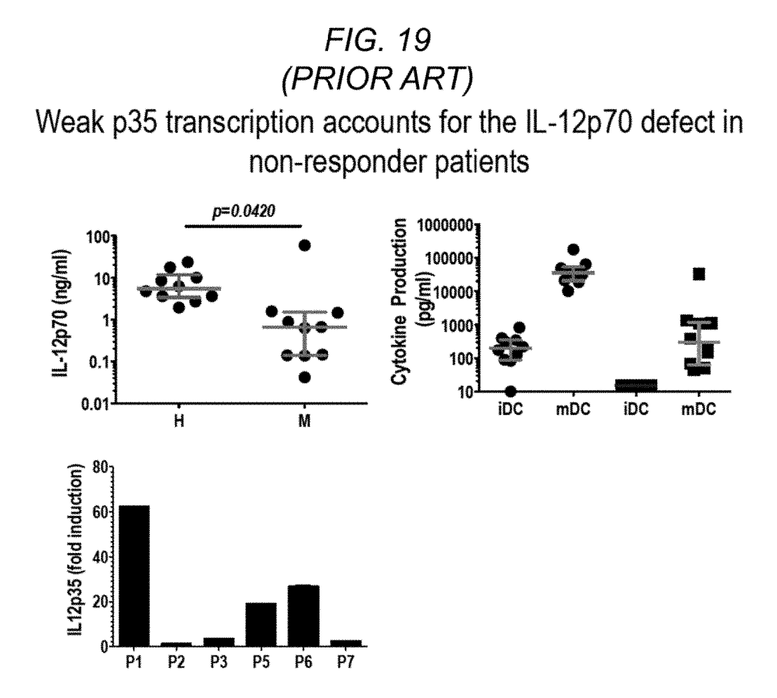

FIG. 19 illustrates that weak p35 transcription accounts for the IL-12p70 defect in clinical non-responder patients (Carreno, B. M., et al., J. Clin. Invest. 123, 3383-3394, 2013). FIG. 19 top, left DC from age and gender matched healthy (H) donors and melanoma (M) patients were activated with CD40L/IFN-γ for 24 h, supernatants harvested and assayed for IL-12 production by ELISA. Horizontal lines and whiskers indicated median and interquartile range. p=0.0420, Wilcoxon matched-pairs test. Healthy individuals;produced on average ˜10× more IL12p70 than melanoma patients. Patient DC were activated with CD40L/IFN-γ for 24 h, supernatants were collected and IL-12p40 (circles) and IL-12p70 (squares) production measured by ELISA (FIG. 19, top right). Results are shown for 10 melanoma patients. Horizontal lines and whiskers indicated median and interquartile range. Results demonstrate a defect on IL-12p70 (p40/p35) but not in IL-12p40 suggesting defect lies in induction of IL-12p35. To examine IL-12p35 gene activation, DC were activated with CD40L/IFN-γ for 6 h, cells harvested, washed and total RNA prepared. Total RNA was also prepared from immature DC. Using p35 and CD11c (DC lineage marker) specific primers, qRT-PCR was performed and analyzed using the relative standard method. Values shown in FIG. 19 (bottom) were normalized to expression CD11c and p35 fold induction in mature DC calculated relative to immature DC. Results decreased IL-12p35 induction in clinical non-responding patients (P2, P3, P7).

### Example 2

This example illustrates techniques of maturing DC that overcome the limitations discussed in Example 1.

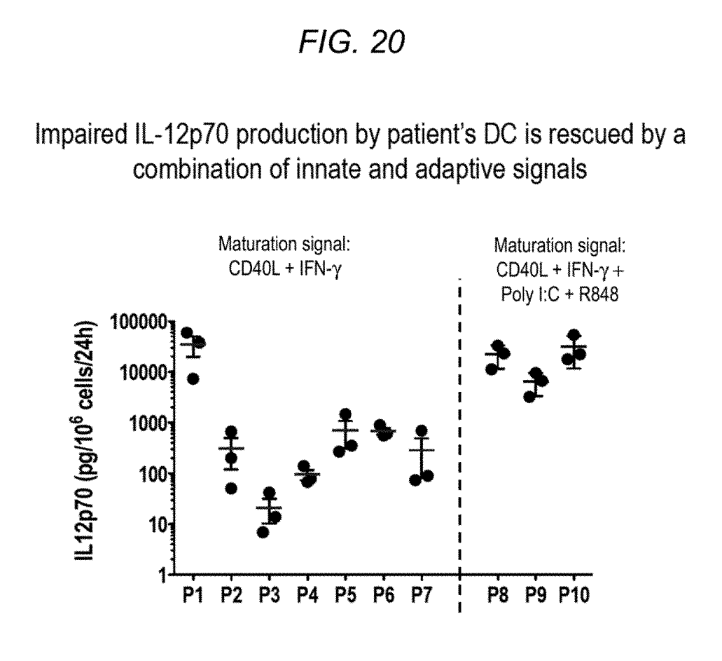

Based on the results obtained in Example 1, different DC maturation techniques were required to increase clinical response to cancer antigens. The inventors therefore tested maturation signals for dendritic cells. Immature DC were stimulated with a combination of CD40L/IFN-γ plus poly I:C (30 ug/mL, TLR3 agonist) and R848 (5 μg/mL, TLR8 agonist) (P8-P10) for 24 h and supernatants assayed for IL-12. As a control, data from immature dendritic cells stimulated with CD40L/IFN-γ(patients P1-P7; Carreno, B. M., et al., J. Clin. Invest. 123, 3383-3394, 2013) were plotted on the same graph. The results depicted in FIG. 20 demonstrate that a combination of all 4 signals enhances IL-12p70 production to levels similar to those observed m healthy individuals (see FIG. 19 top left for the baseline).

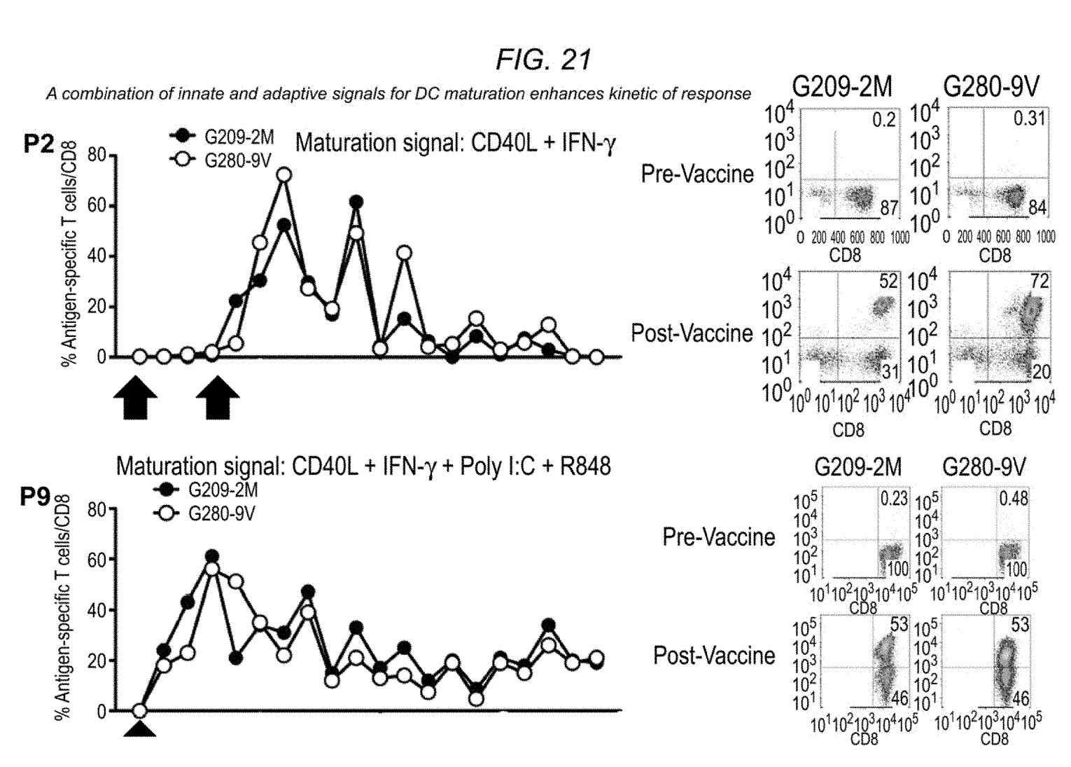

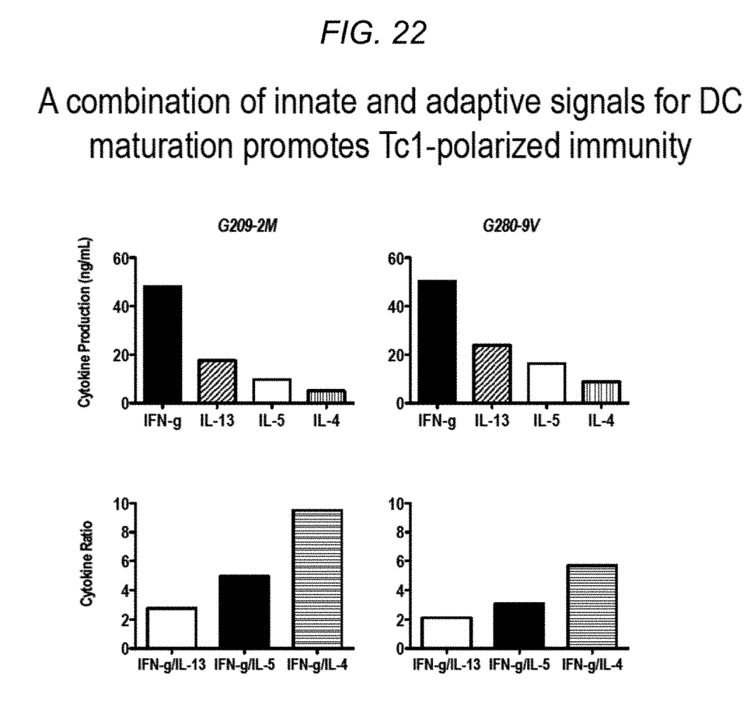

A combination of innate and adaptive signals for DC maturation enhances the kinetics of the immune responses to gp100 (g209-2M and G280-9V) antigens. FIG. 21, left demonstrates that gp100-specific T cell responses can be detected in patients vaccinated with CD40L/IFN-γ/TLR3/8 agonist-matured DC as early as one week after vaccination (bottom left). In contrast, two vaccinations with CD41/IFN-g matured DCs are required for detection of gp100-specific cell responses (FIG. 21, top left). Time is recorded in weeks. Antigen-specific numbers were calculated based on dextramer percentage and total live cell yields. The dot plots (FIG. 21, right) depict frequencies of gp100-specific T cells in ex-vivo expanded peripheral blood mononuclear cells obtained pre- and post-vaccination. FIG. 22 illustrates that a combination of innate and adaptive signals for DC maturation promotes Tc1-polarized immunity. Purified CD8+ T cells were stimulated twice in vitro and antigen-specific frequencies determined by peptide/HLA-A*0201 tetramers. T cells were adjusted to 10⁶ cell/mL, stimulated with antigen and supernatants harvested at 20 h. Cytokine production was determined using MILLIPLEX® MAP Human Cytokine Panel I (FIG. 22, top). To compare production of Tc1 (IFN-γ) and Tc2 (IL-5, IL-13) cytokines among patients, a cytokine ratio was derived by dividing pg/mL IFN-γ by pg/mL IL-5 or IL-13. Ratios>1 indicate a Tc1 phenotype (FIG. 21, bottom).

### Example 3

This example illustrates in silico analysis of missense mutations found in melanoma tumors.

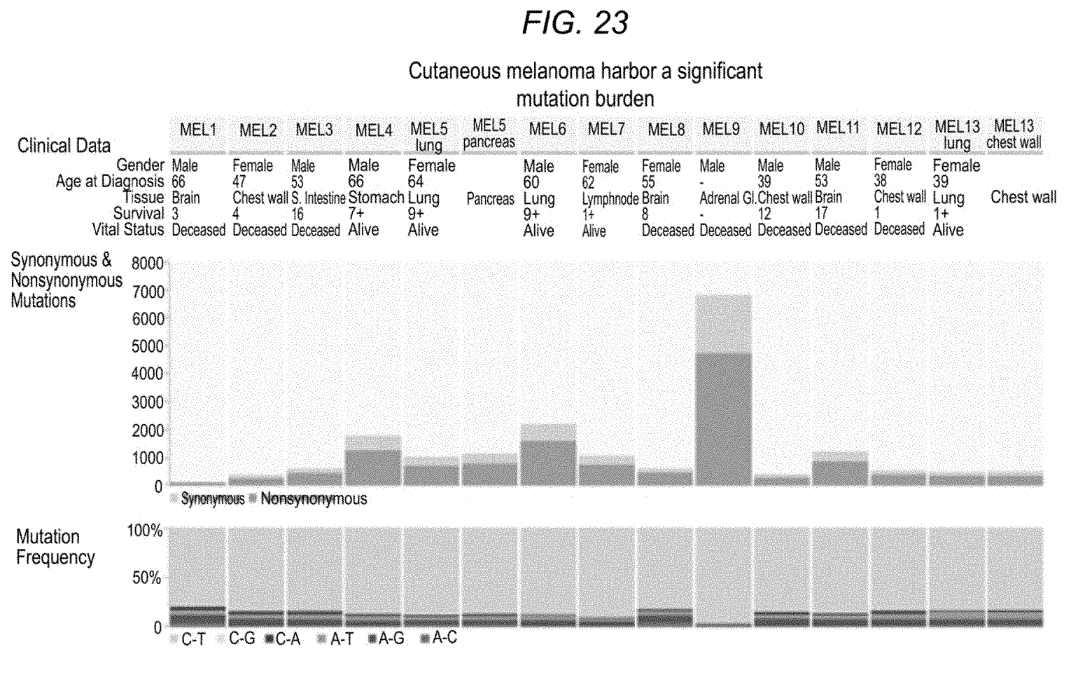

FIG. 23 illustrates that cutaneous melanoma harbors a significant mutation burden and hence continues a cancer model to study tumor somatic mutations as neoantigens. Mutation pattern, spectrum and clinical features in 15 metastases from 13 WGS melanoma cases are illustrated. Numbers and frequencies of Tier 1 transitions and transversions events identified in all 15 tumors are shown. Hence, melanoma patients were chosen for further study of personalized vaccines.

The diagram in FIG. 2 illustrates an example derived from analysis of a tumor/PBMC matched pair derived from a melanoma patient. As depicted multiple candidate patient-specific tumor-derived epitopes can be identified per HLA-class 1 molecule; in this particular case, those presented by HLA-A*0201 are shown. The analysis depicted here can be performed for each of the HLA class I alleles (n=3-6) expressed by the patient.

In various embodiments, the present teachings include analysis of missense mutations by prediction algorithms for binding to HLA-A*0201. Table 1 shows the chromosomal (CHR) location, genomic alignment position and nucleotide change encoding missense mutation in metastases (breast, abdominal wall) derived from a patient. Exomic variant allele fraction (under exome column) for each mutation as well as gene encoding mutation and amino acid change are shown. One mutation in OR5K2 is unique to breast metastasis, while mutations in CCDC57 and IL17Ra are unique to abdominal wall metastasis. Proteins encoding missense mutations were analyzed using the NetMHC and NetMHCstab algorithms in order to predict mutation-containing peptides (9-11 amino acid in length) that may bind to any of patient's HLA-class I molecules. Candidate peptides to consider for a vaccine are selected based on variant frequencies (exome, transcriptome>10), expression (FPKM>1) and HLA class I affinity (<250 nM0 and stability (>2 h). In Table 1, mutated peptides fulfilling these criteria are highlighted in bold. NR=not recorded.

### Example 4

This example illustrates the in vitro binding of neoantigen peptides to HLA class I molecules.

In some embodiments, the present teachings disclose HLA class I binding capacity of peptides containing tumor-specific missense mutations. The binding capacity of missense mutation-containing peptides is experimentally evaluated using a flow cytometric assay. Peptide binding to cell surface HLA class I can lead to stable peptide/HLA class I complexes that can be detected using a HLA-class I allele specific antibody. Four control peptides can be included in the assay, two known HLA-A*0201 binding peptides (FluM1,G280-9V) and 2 negative controls (G17, NP265). In the graph shown in FIG. 3, binding of mutation-containing peptides to HLA-A*0201 expressed on the surface of T2 cells is examined. Nine of the 15 mutation-containing peptides tested bound to HLA-A*0201 and all these peptides show affinities<250 nM.

### Example 5

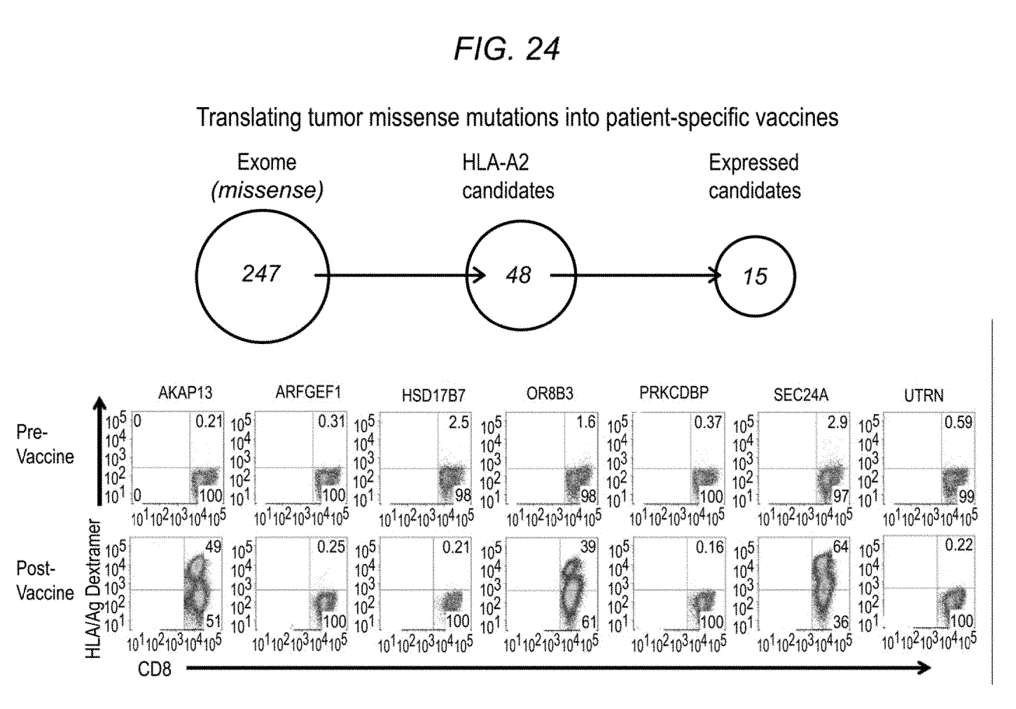

This example illustrates the translation of tumor missense imitations into patient-specific vaccines. FIG. 24 (top) illustrates the distribution of somatic missense mutations identified in a melanoma patient (MEL38) tumor. HLA-A*02:01-binding candidate peptides were in silico identified among amino acid substituted peptides and expression of gene encoding mutated protein determined from cDNA capture data. FIG. 24 (bottom) illustrates the immune-monitoring of neoantigen-specific CD8+ cell responses. Results are derived from PBMC isolated before DC vaccination (Pre-vaccine) and at peak (Post-Vaccine). PBMCs were cultured in vitro in the presence of peptide and IL-2 for 10 days followed by HLA-A*02:01/neoantigen-peptide dextramer assay. This immune monitoring strategy allows the reliable detection, as well as, the assessment of replicative potential of vaccine-induced T cell responses. Numbers within dot plots represent percent neoantigen-specific T cells in lymph+/CD8+ gated cells. A pre-existing response to one neoantigen (SEC24A) was observed; vaccination enhance this response and reveal two additional ones (AKAP13 and OR8B3). Demonstrating that tumor somatic mutations can be immunogenic and that vaccination can expand the antigenic diversity of such response.

### Example 6

This example illustrates CD8+ T cell response to mutation containing peptides.

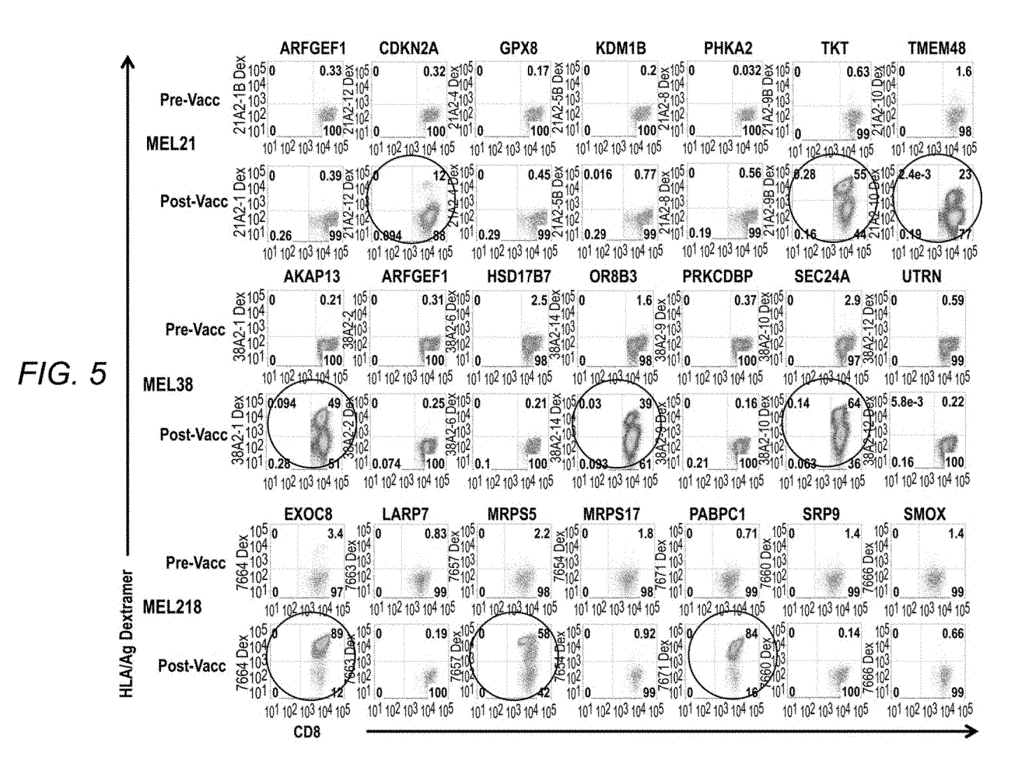

In some embodiments, the present teachings include vaccination with tumor-specific missense mutations to elicit CD8+ T cell immunity. As shown in FIG. 5, a dextramer assay (Carreno, B. M., et al., J. Clin. Invest., 123, 3383-3394, 2013) was used to monitor development of CD8+ T cell immunity to mutation-containing peptides. Dot plots show frequencies of CD8+ T cells specific for the mutation-containing peptides prior to vaccination (pre-vacc) and after 2-3 vaccinations (post-vacc). In all 3 patients, responses to 3 of the 7 peptides are observed as demonstrated by an increase in the frequency of dextramer+ T cells.

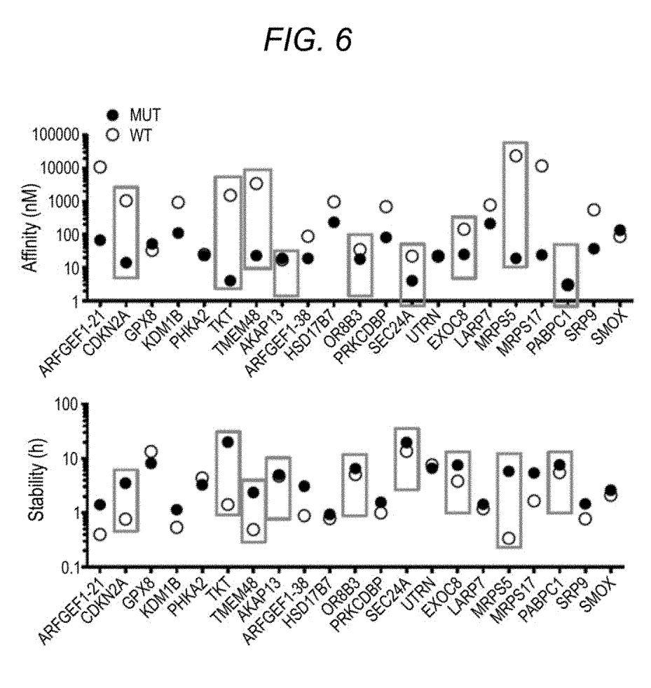

In some embodiments, predicted affinities (FIG. 6 top) and stabilities (FIG. 6 bottom) of mutated peptides and their wild-type counterparts can be compared. In FIG. 6, mutated peptides (two-antigens) that elicited CD+ T cell immunity are indicated by rectangles. All immunogenic peptides display HLA-A*0201 affinities of <50 nM and stabilities>3 h. These characteristics can be important as determinants of immunogenicity. These characteristics can be taken into consideration when choosing mutation-containing peptides to incorporate in a vaccine.

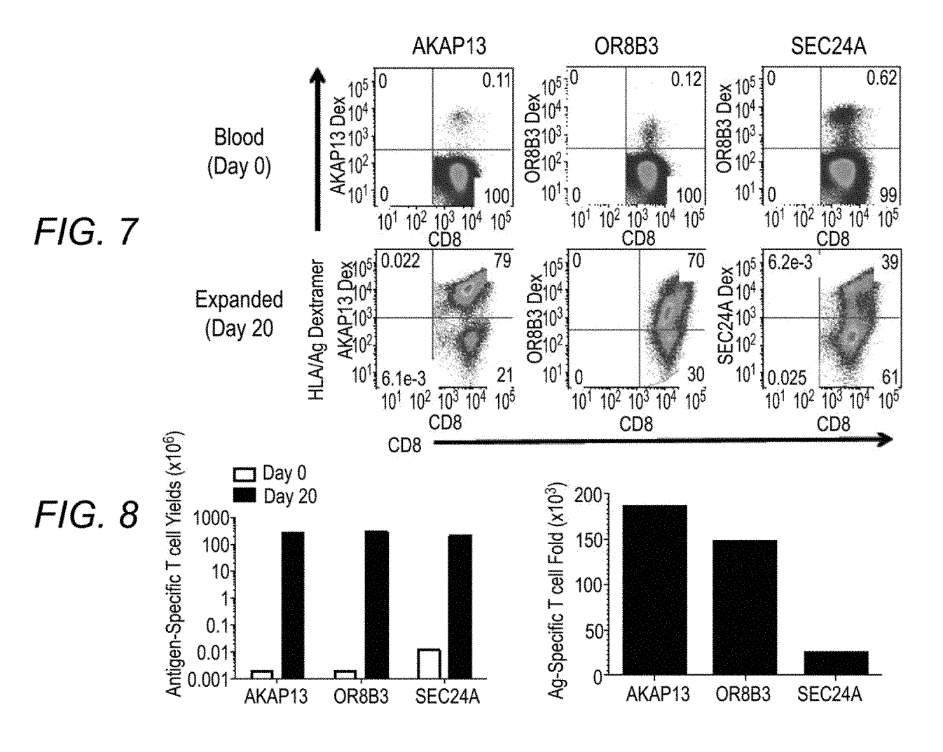

In some embodiments, the present teachings include vaccine-induced CD8+ T cells directed at tumor missense mutations display high replicative potential. As shown in FIG. 7 and FIG. 8, after 3 DC vaccinations, leukapheresis was performed in patients in order to obtain PBMC. CD8+ T cells purified from PBMC were stimulated with neo-antigen-peptide pulsed autologous DC and cultured in the presence of IL-2 for 10 days. These primary cultures were re-stimulated with peptide-pulsed K562-expressing HLA-class I single-chain dimer (SCD) as described (Carreno, B. M., et al., J. Immunol., 188, 5839-5849, 2012). Cultures were maintained for an additional 10 day period in the presence of IL-2. FIG. 7 depicts results from the dextramer assay, the frequencies (%) neo-antigen specific T cells found in the CD8+ T cell population at initiation of cultures (Blood, day 0) and after DC/SCD stimulation (Expanded, day 20) were determined. FIG. 8 illustrates that based on viable cell counts and antigen-specific T cell frequencies, at initiation and termination of cultures, antigen-specific T cell yields and expansion folds were calculated. Antigen-specific yields were calculated as the % of HLA/Ag dextramer+ CD8+ T cells×total CD8+ T cell numbers at day 20. Antigen-specific cell folds represented (% of HLA/Ag dextramer+ CD8+ T cells×total CD8+ T cell numbers at day 20)/(% of HLA/Ag dextramer+ CD8+ T cells×total CD8+ T cell numbers at day 0). Results demonstrated that this method allows the expansion of vaccine-induced T cells over 10⁴ fold (FIG. 8, right panel). A 10⁴ fold expansion yields 10⁸ antigen-specific T cells from a starting population with <10⁴ antigen-specific T cells.

### Example 7

This example illustrates the specificity of neoantigen peptide recognition by CD8+ T cells.

In various embodiments, the present teachings include disclosure of discrimination between mutated and wild-type sequences by vaccine-induced CD8+ T cells.

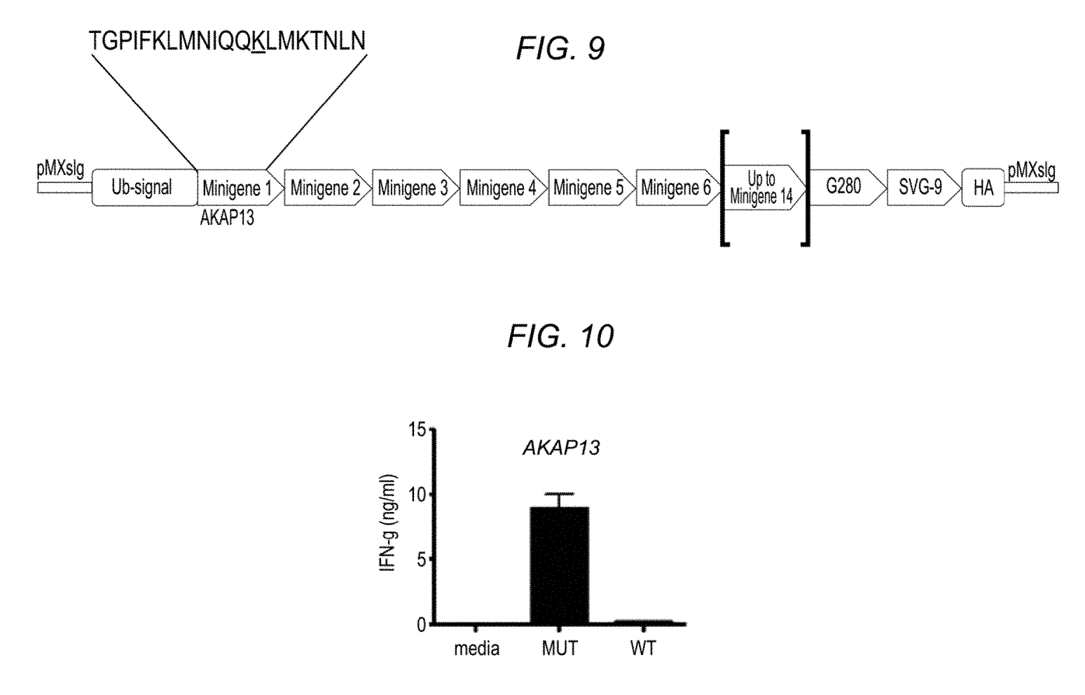

As illustrated in FIG. 9 and FIG. 10, to determine whether vaccine-induced T cells could recognize naturally processed antigen, the melanoma tumor cell line DM6 was transduced with a multi-mini-gene construct encoding mutated (MUT) or wild-type (WT) sequences of peptides incorporated into a vaccine. FIG. 9 illustrates that each minigene consists of 21 aa encoding either the MUT or WT sequences. A scheme depicting minigene construct characteristics and a representative MUT 21-mer aa sequence encoded in construct is shown. Vaccine-induced T cells, specific for AKAP13 containing the Q285K mutation, were incubated with MUT or WT expressing DM6 cells, supernatants collected after 24 h of incubation, and IFN-γ produced by T cells was measured in supernatants by ELISA (FIG. 10). Results indicate that the AKAP13 (Q285K) neo-antigen is processed, presented and recognized by vaccine-induced T cells. The results indicate that a vaccine comprising mutation-containing peptides plus autologous DC can induce cells that will recognize processed and presented antigens on the tumor cell surface.

For therapeutic use of vaccine-induced T cells, it can be important to determine whether responses elicited by MUT peptides can cross-react with WT sequences. T cell responses that cannot discriminate between MUT and WT sequences may have adverse effects if given to patients as part of adoptive cell therapy.

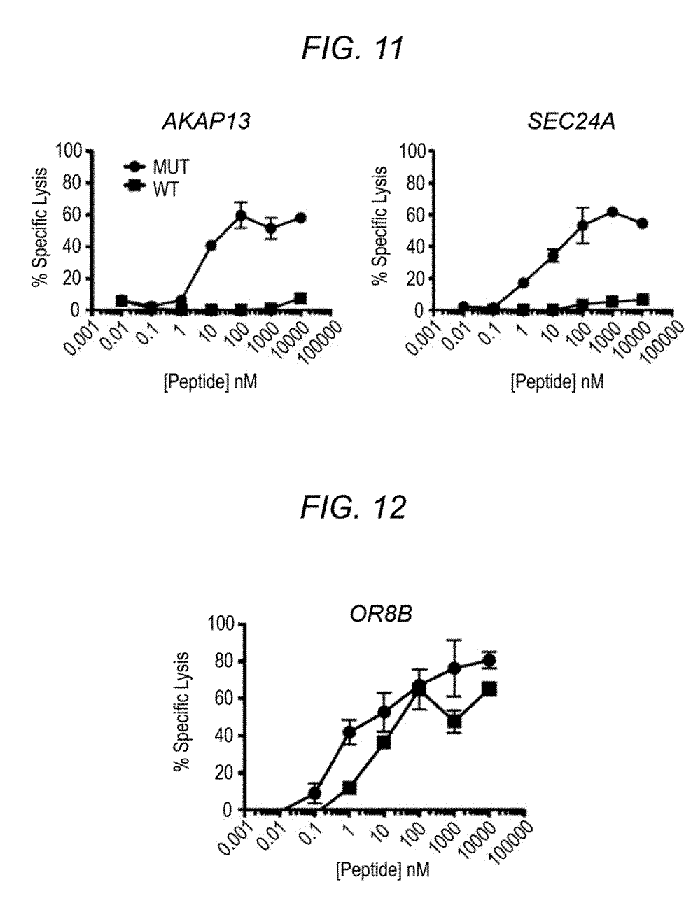

To examine cross-reactivity, T2 cells were pulsed with MUT or WT peptide at the indicated concentrations, labeled with ⁵¹CR-chromium and used as target in a cytotoxic assay. Vaccine-induced T cells were incubated with peptide-pulsed T2 cells and ⁵¹Cr-Chromium release measured at 4 h. Results obtained with T cell lines specific for 3 mutated peptides are shown in FIG. 11-12. The results indicate that T cells can display exquisite antigen specificity and can discriminate between peptide sequence containing single an changes, as shown for AKAP13 and Sec24A (FIG. 11). Only peptides containing the mutated aa can induce lysis of targets. On the other hand, other T cell lines cannot discriminate between MUT and WT sequences as shown for responses directed at OR8B3 (FIG. 12). Thus, screening for cross reactivity can be important in the selection of mutation-specific vaccine-induced T cells to be incorporated in adoptive T cell therapies, only those free of reactivity to WT sequences should be considered.

### Example 8

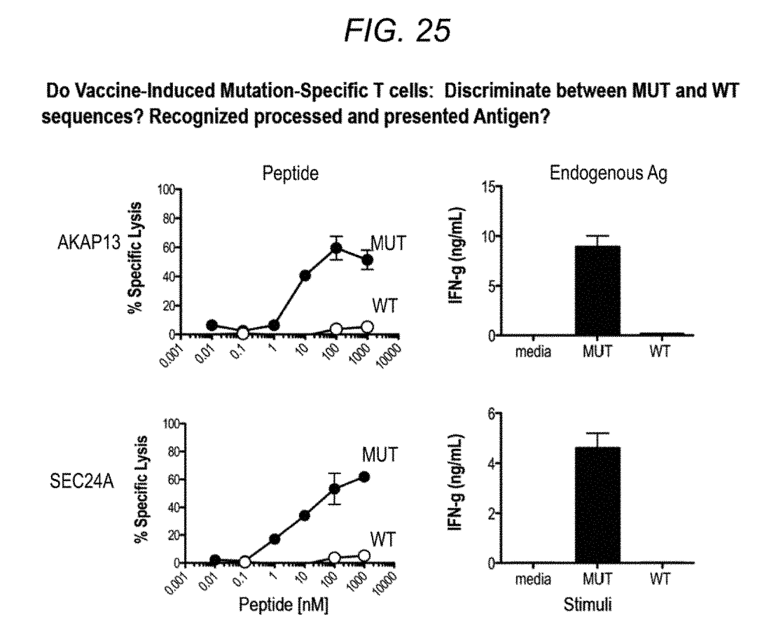

This example illustrates that vaccine-induced mutation-specific T cells discriminate between mutated (MUT) and wild type (WT) sequences and recognized processed and presented antigens. Neoantigen-specific T cells recognition of mutated (closed circles) and wild type (open circles) peptides was determined in a standard 4 h ⁵¹Cr-release assay using peptide titrations on T2 (HLA-A*02:01) cells. Percent specific lysis of triplicates (mean±standard deviation) is shown in FIG. 25 (left) for each peptide concentration; spontaneous lysis was <5%. Results are shown at 10:1 E:T ratio. T cells generated against mutated sequences do not recognize wild-type sequences. Thus, T cells induced by vaccine demonstrate an exquisite specificity for mutated antigen. Neoantigen-specific T cells were co-cultured with DM6 expressing mutated—(closed rectangles) or wild type—(closed circles) tandem mini-gene constructs in a 4 h ⁵¹Cr-release assay. Media represent lysis obtained with parental DM6 cells. Percent specific lysis of triplicates (mean±standard deviation) is shown in FIG. 25 (right) for each E:T ratio; spontaneous lysis was <5%. Therefore, immunization with autologous mature IL-12p70 producing DC elicits shared self-antigen specific T cell responses in humans with cancer. Collectively, these data show that clinical benefit correlates with IL-12p70 which dictates lineage commitment to type-I T cell immunity.

### Example 9

This example illustrates cytokine production in response to neoantigen peptides.

In various embodiments, a vaccine of the present teachings can induce CD8+ T cells to display a Tc1 profile.

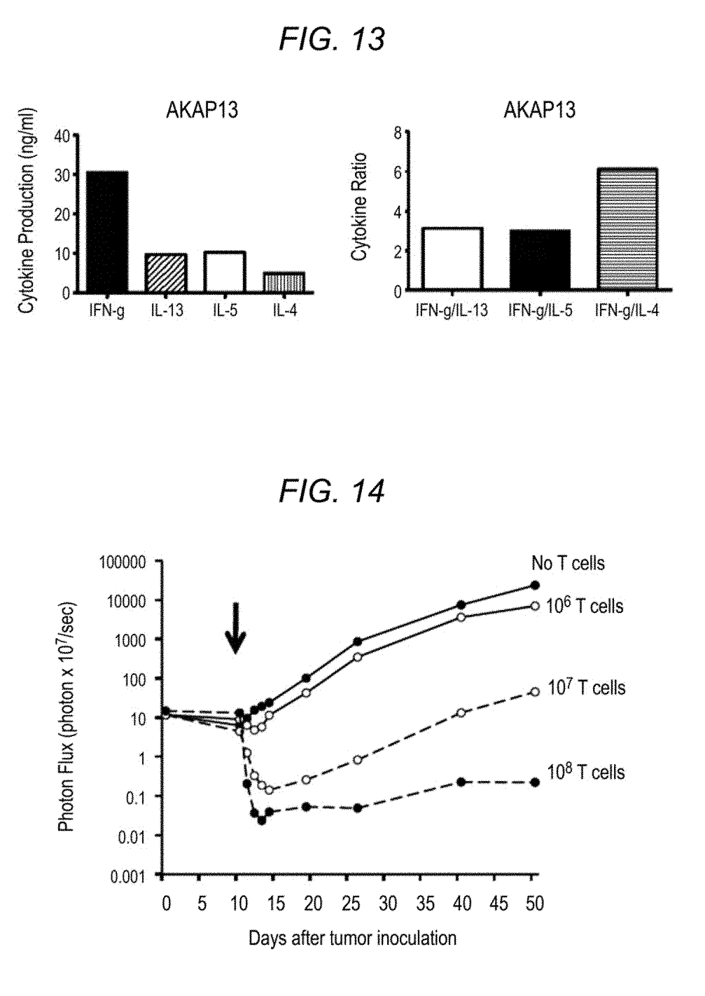

Substantial evidence supports the hypothesis that Th2/Tc2 immune polarization correlates with worse disease outcome in patients with cancer (Fridman, W. H., et al., Nat. Rev. Cancer, 12, 298-306, 2012). In our previous study (Carreno, B. M., et al., J. Clin. Invest., 123, 3383-3394, 2013) the inventors demonstrated that patients presenting vaccine-induced T cells displaying a Tc1 (high IFN-γ, low IL-4, -5, -13 production) benefited from vaccine as determined by an increased time to progression. Thus, we determined production of cytokines upon antigen stimulation as described above. In these studies, neo-antigen-specific AKAP13 (Q285K) T cells were incubated with peptide-pulsed SCD-expressing cells and supernatants collected 24 h after stimulation. Cytokine production was determined using a multi-plex bead assay. Results illustrated in FIG. 13 indicate that vaccine-induced T cells produced large amounts of IFN-γ relative to IL-4, -5 and -13 and hence display a Tc1 phenotype.

### Example 10

This example illustrates successful treatment of melanoma in mice using a vaccine of the present teachings.

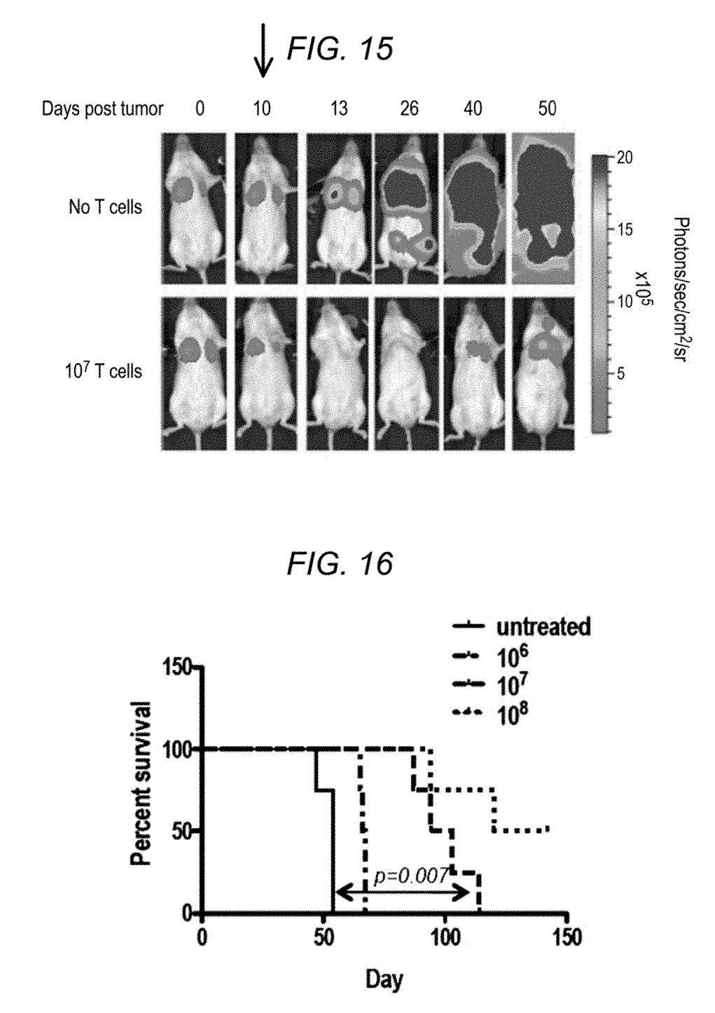

In some embodiments, the present teachings disclose that adoptive transfer of human antigen-specific T cells can lead to melanoma rejection. In investigations by the inventors, humanized mice were inoculated i.v. with luciferase-expressing melanoma. Ten days later (indicated by vertical arrows FIG. 14-15) mice received a single dose of melanoma-specific human T cells (n=5 mice/treatment). FIG. 14 depicts tumor regression monitored by luciferase (photon flux). As shown in FIG. 14 and FIG. 15, in untreated mice luciferase signal increases with time as a result of tumor growth. Conversely in mice treated with T cells, a decrease in luciferase signal was observed. This signal decrease is proportional to the number on cells transferred. These data demonstrate the T cell transfer can result in tumor regression. Importantly, tumor regression can lead to increased survival (FIG. 16). In some configurations, concentration of >10⁷ T cells/mouse can lead to significant changes in survival rates in this model. Adoptive transfer of mutation-specific T cells can lead to tumor regression in this animal model. Furthermore, these pre-clinical results can translate into therapeutic benefit for cancer patients.

### Example 11

This example illustrates selection of neoantigens for further study.

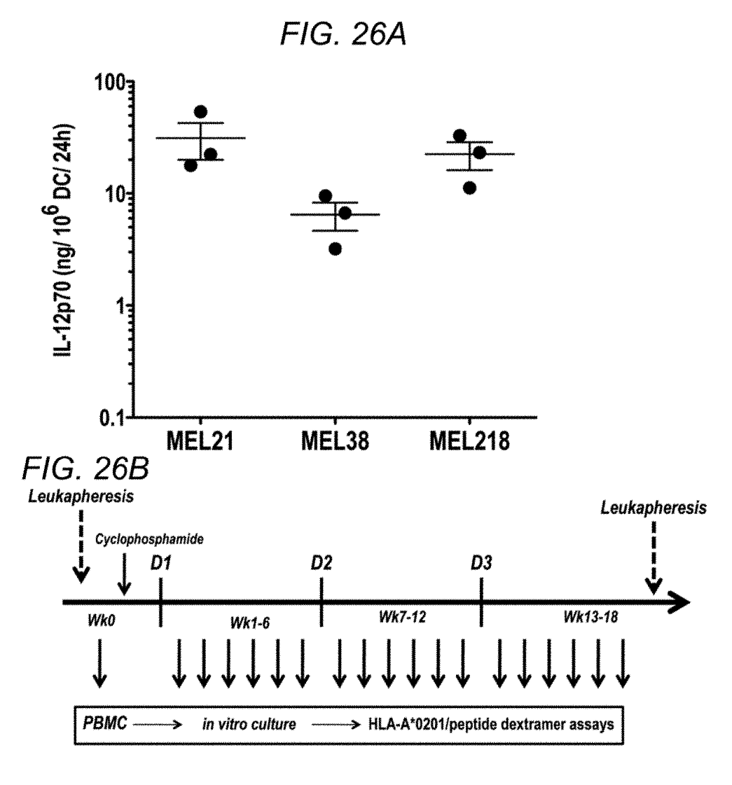

Tumor missense mutations (MM), translated into amino acid substitutions (AAS), may provide a form of antigens that the immune system perceives as foreign, which elicits tumor-specific T cell immunity (Wölfel, T., et al., Science, 269, 1281-1284, 1995; Coulie, P. G., et al., Proc. Nat'l. Acad. Sci. USA 92, 7976-7980, 1995; van Rooij, N. et al., J. Clin. Oncol., 31, e439-e442, 2013; Robbins, P. F., et al., Nat. Med., 19, 747-752, 2013). In these experiments, three patients (MEL21, MEL38 and MEL218) with stage III resected cutaneous melanoma were consented for genomic analysis of their surgically excised tumors and subsequently enrolled in a phase 1 clinical trial with autologous, functionally mature, interleukin (IL)-12p70-producing dendritic cell (DC) vaccine (FIG. 26A-B) (Carreno, B. M., et al., J. Clin. Invest., 123, 3381-3394, 2013). FIG. 26A illustrates that dendritic cells (DC) were matured with CD40L, IFN-γ plus TLR3 (poly I:C) and TLR8 (R848) agonists in order to optimize the production of IL-12p70. Results shown are the ex-vivo IL-12p70 levels produced by patient-derived mature DC used for manufacturing vaccines doses D1-D3 (each symbol represents a vaccine dose). DC supernatants were harvested 24 h after activation and IL-12p70 production levels determined by ELISA. Results represent mean±SEM. FIG. 26B illustrates that study timelines depicting cyclophosphamide treatment (300 mg/m² i.v), DC vaccinations (D1-D3), PBMC sampling for immune monitoring and leukapheresis collections. The vaccine dosing schedule was altered from every 3 weeks to every 6 weeks based on the kinetics of the T cell response previously reported (Carreno, B. M., et al., J. Clin. Invest., 123, 3383-3394, 2013).

All tumor samples were flash frozen except one from MEL 21 (skin, Jun. 6, 2013), which was formalin-fixed paraffin embedded. Peripheral blood mononuclear cells (PBMC) were cryopreserved as cell pellets. DNA samples were prepared using QIAAMP® DNA Mini Kit (Qiagen) and RNA using High Pure RNA Paraffin kit (Roche), DNA and RNA quality was determined by NANODROP® 2000 and quantitated by the QUBIT® Fluorometer (Life Technologies). For each patient, tumor/PBMC (normal) matched genomic DNA samples were processed for exome sequencing with one normal and two tumor libraries, each using 500 ng DNA input (Service, S. K. et al., P.L.o.S. Genet., 10, e1004147, 2014). Exome sequencing was performed to identify somatic mutations in tumor samples.

Tumor M M, translated as AAS-encoding nonamer peptides, were filtered through in silico analysis to assess HLA-A*02:01 peptide binding affinity (Nielsen, M. et al., Protein Sci., 12, 1007-1017, 2003). Alignment of exome reads was performed using the inventors' Genome Modeling System (GMS) processing-profile. This pipeline uses BWA (version 0.5.9) for alignment with default parameters except for the following: ‘−t 4 −q 5’. All alignments were against GRCh37-lite-build37 of the human reference genome and were merged and subsequently de-duplicated with Picard (version 1.46). Detection of somatic mutations was performed using the union of three variant callers: 1) SAMtools version r963 (params: −A −B) filtered by snp-filter v1 and further intersected with Somatic Sniper version 1.0.2 (params: −F vcf q 1 −Q 15) and processed through false-positive filter v1 (params: -bam-readcount-version 0.4-bamreadcount-min-base-quality 15 min-mapping-quality 40-min-somatic-score 40) 2) VarScan Somatic version 2.2.6 filtered by varscan-high-confidence filter version v1 and processed through false-positive filter v1 (params, -bam-readcount-version 0.4bamreadcount-min-base-quality 15), and 3) Strelka version 1.0.10 (params: isSkipDepthFilters=1). Amino acid substitutions (AAS) corresponding to each of the coding missense mutations (MM) were translated into a 21-mer amino acid FASTA sequence, with ideally 10 amino acids flanking the substituted amino acid on each side.

Each 21-mer amino acid sequence was then evaluated through the HLA class I peptide binding algorithm NetMHC 3.4 to predict high affinity HLA-A*02:01 nonamer peptides for the AAS—as well as the WT sequence to calculate differences in binding affinities (8, 32). Any peptides with binding affinity IC₅₀ value<500 nM were considered for further analysis.

Experimental expression of genes encoding predicted HLA-A*02:01 peptide candidates was determined by cDNA capture. All RNA samples were DNase-treated with TURBO DNA-FREE™ kit (Invitrogen) according to the manufacturer's instructions; RNA integrity and concentration were assessed using Agilent Eukaryotic Total RNA 6000 assay (Agilent Technologies) and QUANT-IT™ RNA assay kit on a QUBIT™ Fluorometer (Life Technologies Corporation).

Given the dynamic nature of genomic technologies, multiple overlapping methods were tested. However, results for tumors within a patient (Tables 2-4) are consistent with one methodology: NuGen OVATION® V2 for MEL38 and MED218, Illumina TRUSEQ® Stranded for MEL21. The MicroPoly(A)PURIST™ Kit (Ambion) was used to enrich for poly(A) RNA from MEL218 and MEL38 DNAse-treated total RNA; MEL21 RNA was ribo-depleted using the RIBO-ZERO™ Magnetic Gold Kit (EpiCeture, Madison Wis.) following the manufacturer protocol. The inventors used either the OVATION® RNA-Seq System V2 (NuGen, 20 ng of either total or polyA RNA), or the OVATION® RNA-Seq FFPE System (NuGen, 150 ng of DNase-treated total RNA) or the TRUSEQ® Stranded Total RNA Sample Prep kit (Illumina, 20 ng ribosomal RNA-depleted total RNA) for cDNA synthesis. All NuGen cDNA sequencing libraries were generated using NEBNEXT® ULTRA™ DNA Library Prep Kit for ILLUMINA® with minor modifications.

All NuGEN generated cDNA was processed as described previously (Cabanski, C. R., et al., J. Mol. Diagn., 16, 440-451, 2014). Briefly, 500 ng of cDNA was fragmented, end-repaired, and adapter-ligated using IDT synthesized “dual same index” adapters. The TRUSEQ® stranded cDNA was also end-repaired and adapter-ligated using IDT synthesized “dual same index” adapters. These indexed adapters, similar to Illumina TRUESEQ® HT adapters, contain the same 8 bp index on both strands of the adapter. Binning reads requires 100% identity from the forward and reverse indexes to minimize sample crosstalk in pooling strategies. Each library ligation reaction was PCR-optimized using the Eppendorf Epigradient SqPCR instrument, and PCR-amplified for limited cycle numbers based on the Ct value in the optimization step.

Libraries were assessed for concentration using the QUANT-IT™ dsDNA HS Assay (Life Technologies) and for size using the BioAnalyzer 2100 and the Agilent DNA 1000 Assay (Agilent Technologies). The ILLUMINA®-ready libraries were enriched using the Nimblegen SeqCap EZ Human. Exome Library v3.0 reagent. The targeted genomic regions in this kit cover 63.5 Mb or 2.1% of the human reference genome, including 98.8% of coding regions, 23.1% of untranslated regions (UTRs), and 55.5% of miRNA bases (as annotated by Ensembl version 73 (Flicek. P., et al., Nucleic Acids Res., 41, D48-55, 2013)). Each hybridization reaction was incubated at 47° C. for 72 hours, and single-stranded capture libraries were recovered and PCR-amplified per the manufacturer's protocol. Post-capture library pools were sized and mixed at a 1:0.6 sample: Ampure XP magnetic head ratio to remove residual primer-dimers and to enrich for a library fragment distribution between 300 and 500 bp. The pooled capture libraries were diluted to 2 nM for Illumina sequencing.

For cDNA-capture data were aligned with Tophat v2.0.8 (params: version=2.1.0 for OVATION®; -library-type fr-firststrand-bowtie-version=2.1.0 for TRUSEQ®). For OVATION® data, prior to alignment, paired 2×100 bp sequence reads were trimmed with flexbar v 2.21 (params: -adapter CTTTGTGTTTGA (SEQ ID NO: 474-adapter-trim-end LEFT-nono-length-dist-threads 4-adapter-min-overlap 7-maxuncalled 150-min-readlength 25) to remove single primer isothermal amplification adapter sequences. In seqcap, the relative expression of a transcript is proportional to the number of cDNA fragments that originate from it. Therefore, expression levels expressed as fragments per kilobase of exon per million fragments mapped (FPKM) were calculated with Cufflinks v2.0.2 (Trapnell et al. 2010, Nature Biotechnology 28, 511; params-max-bundle-length=10000000-num-threads 4). A visual review step of cDNA capture data was performed to evaluate for expression of MM identified by exome data. Both cDNA-capture and FPKM values were considered for candidate prioritization.

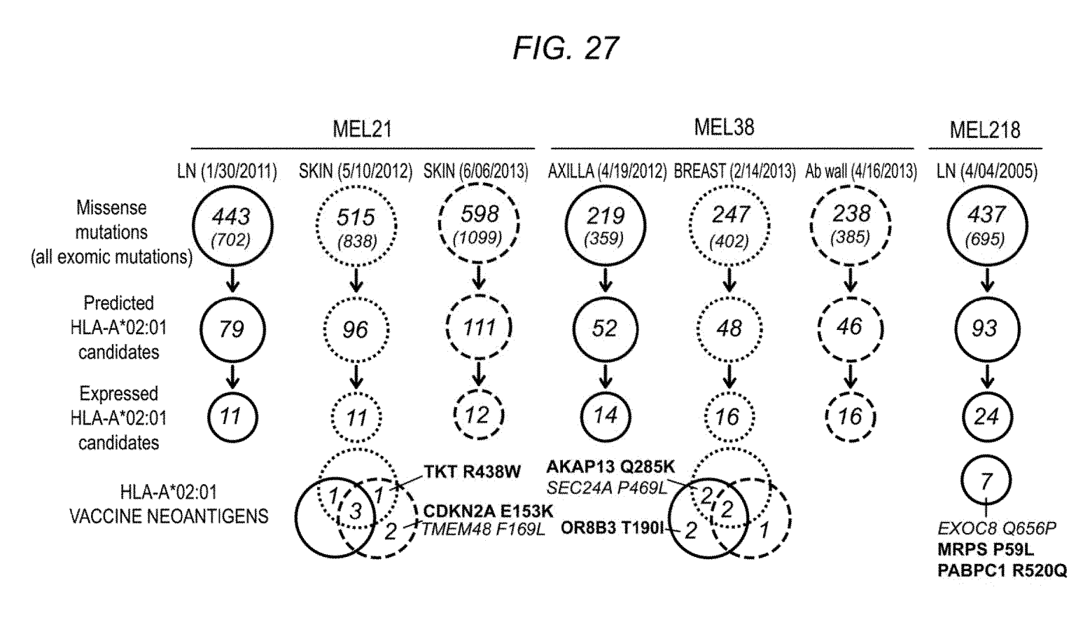

FIG. 27 illustrates distribution of somatic (exomic and missense) mutations identified in patients MEL21 and MEL38 metachronous tumors (anatomical location and date of collection indicated) and patient MEL218 tumor are shown. HLA-A*02:01-binding candidate peptides were identified among AAS and expression of gene encoding mutated protein determined from cDNA capture data (Tables 2-4) as discussed supra. Venn diagrams show expression, among metachronous tumors, of mutated genes encoding vaccine neoantigens. The identities of the three immunogenic neoantigens identified in each patient are depicted in diagrams; type style identifies naturally occurring (italics) and vaccine-induced (bold) neoantigens.

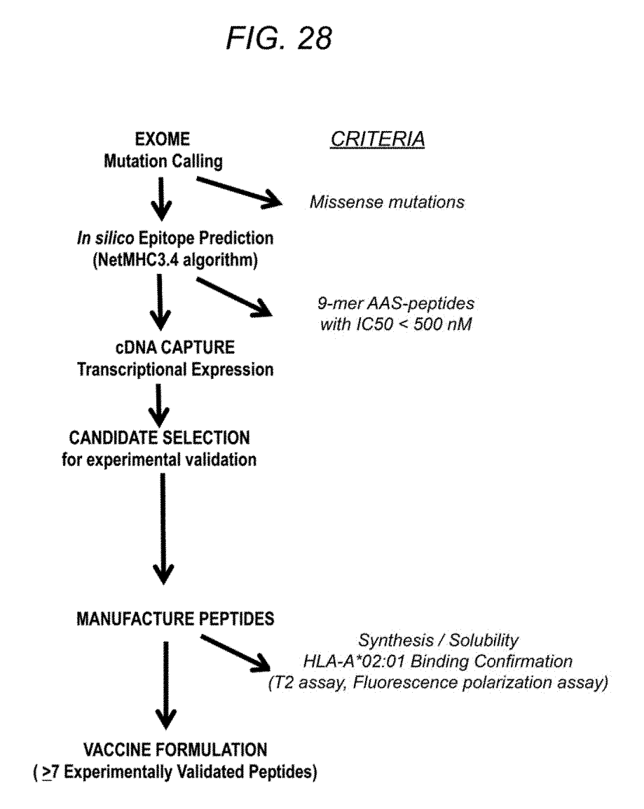

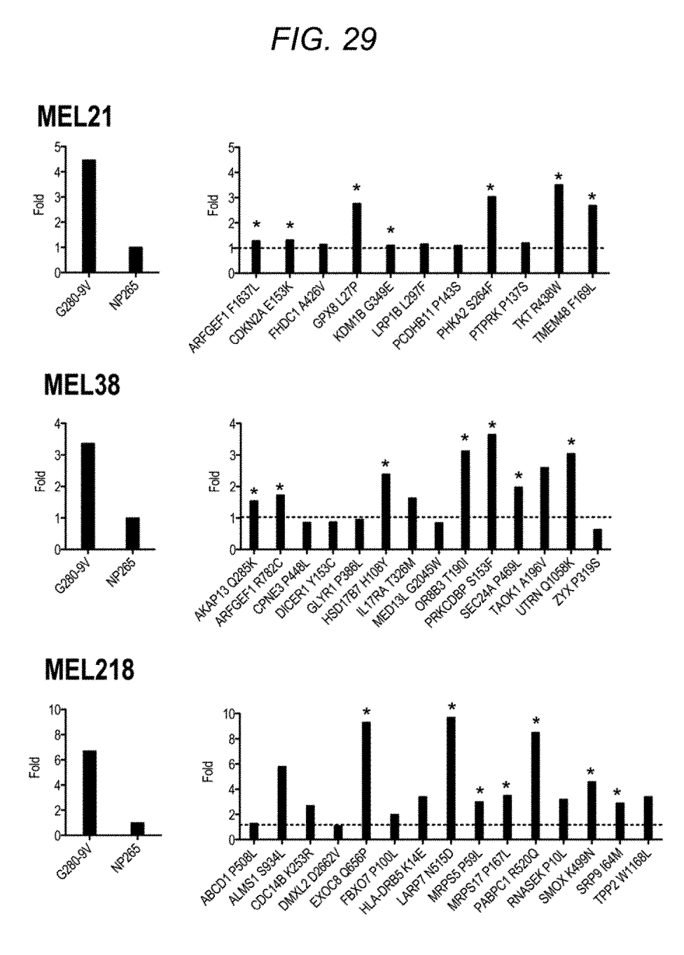

Peptide candidates for experimental validation were selected according to the strategy described in FIG. 28: Tumor-specific missense mutations (MM) in melanoma samples were detected using exome sequencing and identified using the union of three variant calling algorithms. BRAF allelic frequency (Tables 2-4) was considered the upper limit variant allelic fraction fix each tumor and used as a comparator to assess the clonality of other MM-encoding genes. Amino acid substitutions (AAS) corresponding to each of the coding MM were translated into a 21-mer amino acid FASTA sequence and evaluated through the HLA class I peptide binding algorithm NetMHC 3.4 to predict HLA-A*02:01 nonamer AAS-encoding peptides with EC₅₀<500 nM. Transcriptional status of genes encoding AAS candidates was determined by cDNA-capture and their expression levels determined using Cufflinks. Filters were applied to deprioritize those with low cDNA-capture (Alt_reads<5) and prioritized those with high numbers of Atl_reads and/or FPKM>1. For MEL21 and MEL38 patients, candidates were prioritized if expressed by more than one metachronous tumor. For experimental validation, candidates were further prioritized on the basis of predicted HLA-A*02:01 binding affinity and/or HLA-A*02:01 affinity differential between AAS- and WT-peptide (Tables 2-4). Only those peptides with confirmed HLA-A*02:01 binding as determined by T2 assay (FIG. 29) and fluorescence polarization assay [log(IC₅₀ nM)<4.7, Table 5]were prioritized for vaccine formulation.

HLA-A*02:01 binding was evaluated using the T2 assay (See Analysis of T cell responses) (FIG. 29) (Elvin, J., et al., J. Immunol. Methods, 158, 161-171, 1993) and confirmed in the fluorescence polarization-based competitive peptide binding assay (Buchli, R., et al., Biochemistry, 44, 12491-12507, 2005). FIG. 29 illustrates AAS-encoding peptide binding to HLA-A*02:01. T2 cells were incubated with 100 uM of the indicated peptide for 16 h, washed and stained with PE-conjugated anti-HLA-A*02:01 (clone BB7.2) monoclonal antibody. Melanoma G280-9V and Influenza NP265 peptides represent positive and negative controls, respectively. Binding fold are calculated as MFI experimental peptide/MFI NP265 peptide. Data are representative of 3 independent experiments. Peptides selected for incorporation in the vaccine formulation are indicated with an asterisk. Per patient, 7 AAS peptide candidates were selected among validated HLA-A*02:01 binders (Table 5) for incorporation into a personalized vaccine formulation along with the melanoma gp100-derived peptides G209-2M and G280-9V (as positive controls for vaccination) (Carreno, B. M., et al., J. Clin. Invest., 123, 3383-3394, 2013). The expression pattern of mutated genes encoding vaccine candidates is shown in Venn diagrams in FIG. 27.

### Example 12

This example illustrates the effectiveness of personalized dendritic vaccines.

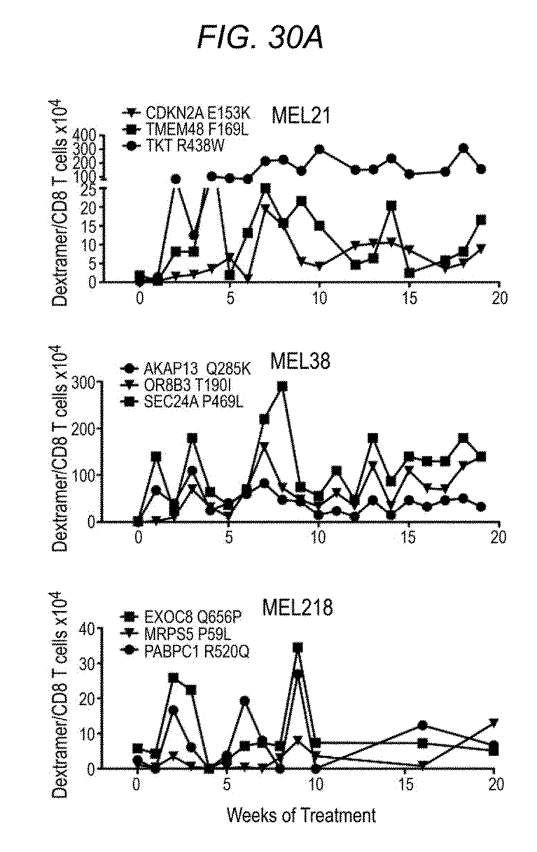

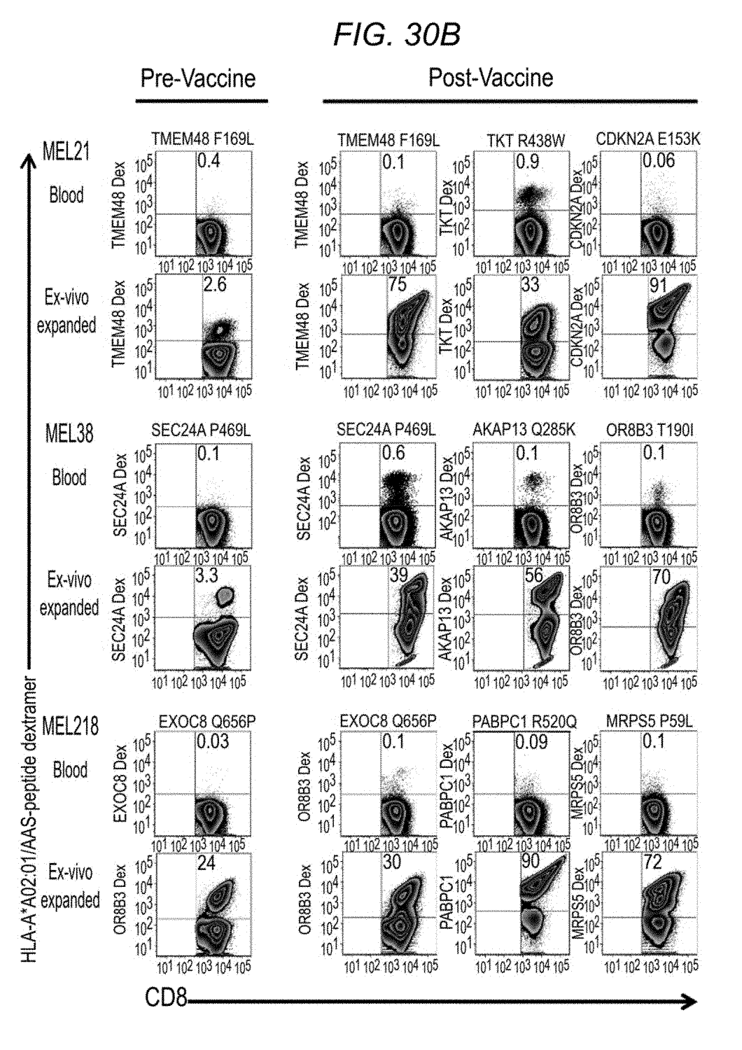

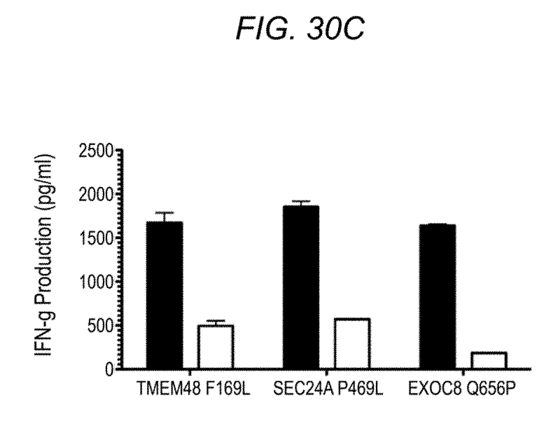

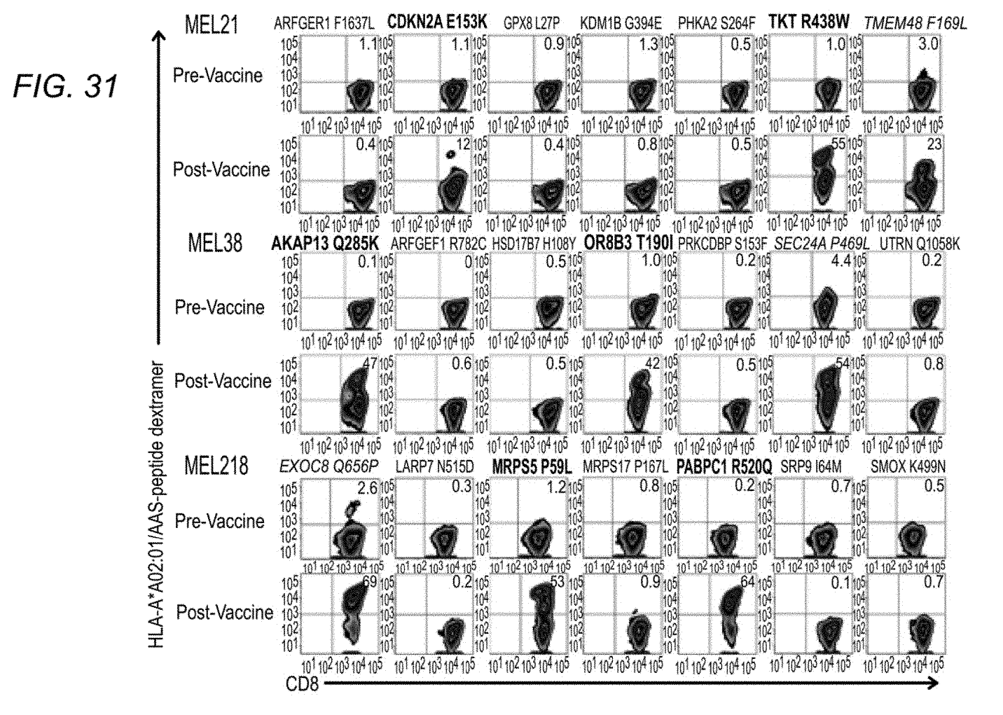

To examine the kinetics and magnitude of T cell immunity to AAS peptides upon vaccination, peripheral blood mononuclear cells (PBMC) were collected prior to vaccination and weekly thereafter. The CD8+ T cell response to each peptide was analyzed using a HLA-A*02:01/AAS-peptide dextramer assay after a single round of in vitro stimulation. FIG. 30A illustrates kinetics of immune responses to neoantigens. Time is recorded in weeks (0 indicates pre-vaccination). Culture conditions and staining details are described infra. Antigen-specific numbers were calculated based on dextramer percentage and total live cell yields. Immunologic analysis to evaluate the kinetic and magnitude of T cell response to AAS-encoding and gp100-derived peptides was performed using PBMC collected weekly, starting before DC vaccination (Pre-vaccine in the figures) as described (Carreno, B. M., et al., J. Clin. Invest., 123, 3383-3394, 2013). Briefly, fresh PBMC obtained by Ficoll-Paque PLUS gradient centrifugation were cultured with 40 ug/mL peptide and IL-2 (50 U/mL). On day 10 (peak of response, unpublished data, labeled “Post-Vaccine” in the figures), neoantigen specific T cell frequencies were determined by staining with HLA-A*02:01/peptide dextramers (Immudex), followed by addition of FITC-CD4, -CD14, -CD19 (Invitrogen) and ALEXA® 488-CD56 (BD Pharmigen), APC-CD8 (Invitrogen). Cells were washed, resuspended in FACS buffer containing 7AAD. Twenty five thousand events in the CD8+ gate were collected using a hierarchical gating strategy that included FSC/SSC and excluded 7AAD-positive (dead cells) and CD4/14/19/56-positive cells. PBMC/CD8+ T cells derived from an unrelated HLA-A*02:01 patient were used as negative controls for assessing specificity of HLA-A*02:01/AAS-peptide dextramers (data not shown). Data were acquired and analyzed using Flow-Jo software. Immune monitoring demonstrated that in each patient, T cell immunity to one AAS peptide could be detected in pre-vaccine PBMC samples after in vitro stimulation (FIG. 31, MEL21:TMEM48 F169L; MEL38; SEC24A P469L and MEL218: EXOC8 Q656P, type style identifies naturally occurring (italics) and vaccine-induced (bold) neoantigens) although not directly from the blood. FIG. 30B illustrates the frequency of neoantigen specific T cells in CD8+ populations isolated directly from PBMC samples and after ex-vivo expansion using autologous DC and artificial antigen presenting cells. For dominant neoantigens TMEM48 F169L, SEC24A P469L and EXOC8 Q656, results are shown for samples obtained before vaccination (Pre-vaccine) and after 3 vaccine doses (Post-vaccine). For remaining neoantigens, results obtained with post-vaccine PBMC samples are shown. Percentage of neoantigen-specific CD8+ T cells is indicated in the right upper quadrant of the plot. A representative experiment of two performed is shown. Pre-existing immunity to these three neoantigens was confirmed in ex-vivo expanded pre-vaccine purified CD8+ T cells using dextramer assay (FIG. 30B) and interferon (IFN)-γ production. FIG. 30C illustrates ex-vivo expanded pre-vaccine neoantigen-specific T cells (dextramer % shown in FIG. 30B) were stimulated with artificial antigen presenting cells in the presence (closed bar) or absence (open bar) of AAS-peptide and supernatants were harvested at 24 h. IFN-γ production was determined using ELISA assay. Mean values+/−standard deviation (SD) of duplicates are shown. Cytokine production by T cells in the absence of any stimuli was <100 pg/mL.

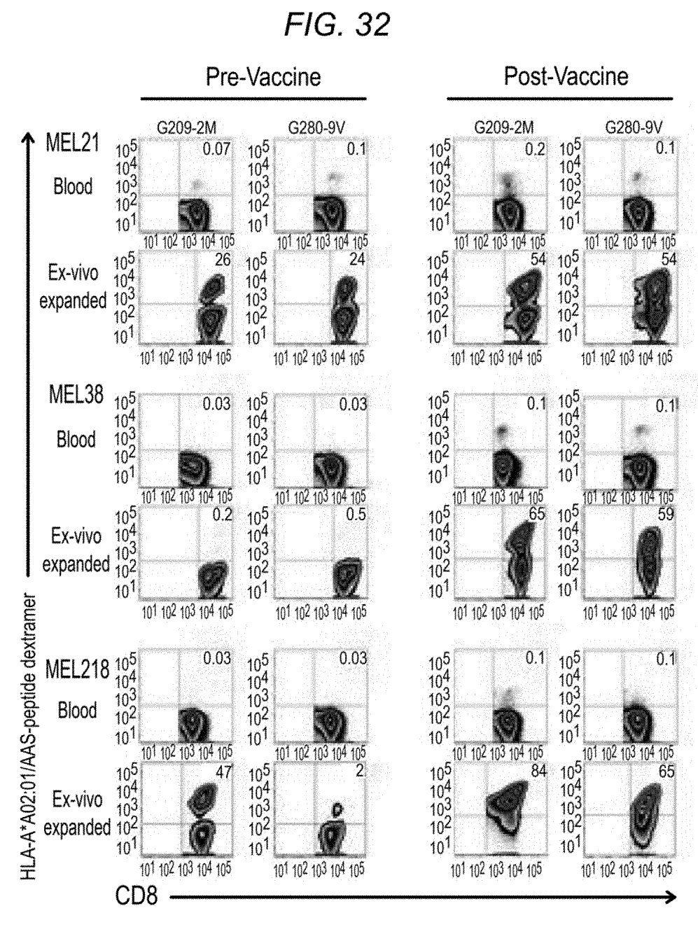

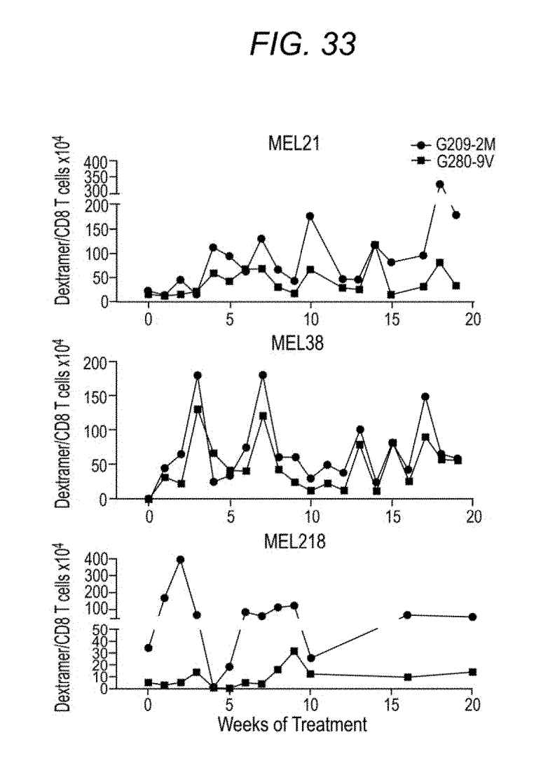

Vaccination augmented the cell response to these neoantigens with observed frequencies of 23% TMEM48 F169L+ CD8+ T cells, 64% SEC24A P469L+ CD8+T cells and 89% EXOC8 Q656P+ CD8 T cells detected, upon culture, at the peak of response (FIG. 31). Immune monitoring also revealed vaccine-induced T cell immunity to two additional neoantigens per patient: TKT R438W and CDKN2A E153K (55% and 12%, respectively) in patient MEL21; AKAP13 Q285K and OR8B3 T190I (47% and 42%, respectively) in patient MEL38, and MRPS5 P59L and PABC1 R520Q (58% and 84%, respectively) in patient MEL218 (FIG. 31). Two (MEL21 and MEL218) of the three patients had pre-existing immunity to G209-2M and G280-9V peptides, as determined by the presence of gp100-specific T cells in pre-vaccine PBMC samples and their ex-vivo expansion upon antigen stimulation. FIG. 32 illustrates the frequency of G209-2M- and G280-9V-specific T cells in CD8+ populations isolated directly from PBMC samples and after ex-vivo expansion using autologous DC and artificial antigen presenting cells. Results are shown for samples obtained before vaccination (Pre-vaccine) and at peak post vaccination (Post-vaccine). Percentage of antigen-specific CD8+ T cells is indicated in the right upper quadrant of the plot. A representative experiment of three performed is shown. Upon vaccination, these T cell responses were enhanced in patients MEL21 and MEL218 and revealed in patient MEL38. FIG. 33 illustrates the kinetics of immune responses to G209-2M and G280-9V peptides. Time is recorded in weeks (0 indicates prevaccination). Culture conditions and staining details are described supra. Antigen specific numbers were calculated based on dextramer percentage and total live cell yields. No T cell immunity was detected to the remaining 12 AAS peptides. Overall, robust neoantigen T cell immunity was detectable as early as week 2 and peaked at week 8-9 after the initial vaccine dose (FIG. 30A). Neoantigen-specific CD8+ T cells are readily identified by dextramer assay directly in post-vaccine PBMC samples (FIG. 30B) and memory T cells are detected up to 4 months after the final vaccine dose.

Analysis of T cell reactivity among the three patients indicated no preferential skewing towards AAS at specific positions in the peptide sequence—that is towards TCR, contact residues or primary anchor residues (Kim, Y., et al., J. Immunol. Methods, 374, 62-69, 2011). Rather, in each patient, T cell immunity appeared to focus on the 3 AAS candidates exhibiting the highest HLAA*02:01 binding affinity while the remaining medium-high affinity peptides were nonimmunogenic (Table 5) (Nielsen M., et al., Protein Sci., 12, 1007-1017, 2003; Buchli, R., et al., Biochemistry, 44, 12491-12507, 2005). Immunogenic AAS peptides (FIG. 27) were not preferentially derived from genes with high allelic frequency or expression levels (Tables 2-4).

To characterize the function of vaccine-induced neoantigen-specific T cells, short-term expanded CD8+ T cell lines were established and antigen specificity confirmed by dextramer assay (FIG. 30B) (Carreno B. M., et al., J. Clin. Invest., 123, 3383-3394, 2013; Carreno, B. M. et al., J. Immunol, 188, 5839-5849, 2012). Neoantigen-specific T cell lines were generated using autologous mDC and antigen loaded artificial antigen presenting cells at a ratio of 1:1 as previously described (Carreno, B. M. et al., J. Immunol. 188, 5839-5849, 2012); antigen-specific frequencies in cell lines are shown in FIG. 30B. To determine the peptide avidity (effective concentration at 50% maximal lysis, EC₅₀) of neoantigen-specific T cells, T2 cells were pulsed with titrated peptide concentrations for 1 h, followed by ⁵¹Cr (25μCi) labeling for 1 h, washed twice and tested in a standard 4 h ⁵¹Cr release assay using neoantigen-specific T cells as effectors. For production of cytokines, neoantigen-specific T cells were restimulated using artificial antigen presenting cells in the presence or absence of peptide, supernatants collected at 24 h and cytokine produced determined using MILLIPLEX® MAP Human Cytokine Panel I (EMD Millipore).

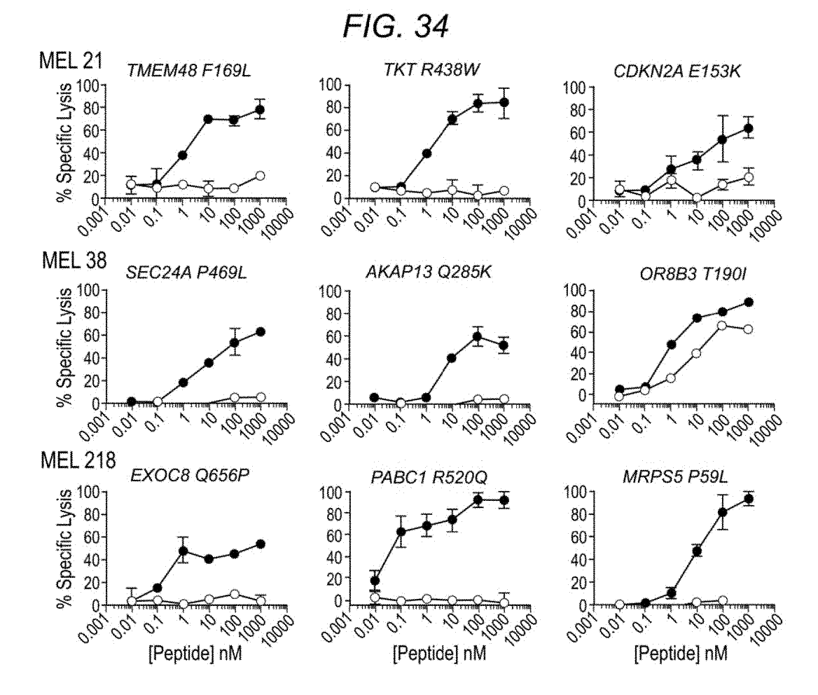

FIG. 34 illustrates that neoantigen-specific T cells recognition of AAS (closed circles) and WT (open circles) peptides was determined in a standard 4 h ⁵¹Cr-release assay using peptide titrations on T2 (HLAA*02:01) cells. Percent specific lysis of triplicates (mean+standard deviation) is shown for each peptide concentration; spontaneous lysis was <5%. Results are shown at 10:1 E:T ratios for all cell lines except TMEM48 F169L and CDKN2A E153K cells which are shown at 60:1 E:T ratio. A representative experiment of two independent evaluations is shown. Neoantigen-specific T cells displayed significant levels of cytotoxic activity at AAS peptide concentrations of 1 to 10 nM, a finding that is consistent with high avidity cell recognition of antigen (FIG. 34). OR8B3 T190I-specific T cells could not discriminate between AAS and wild-type (WT) peptide when presented on T2 cells, while all of the remaining cell lines showed clear specificity for AAS peptide sequences (FIG. 34).

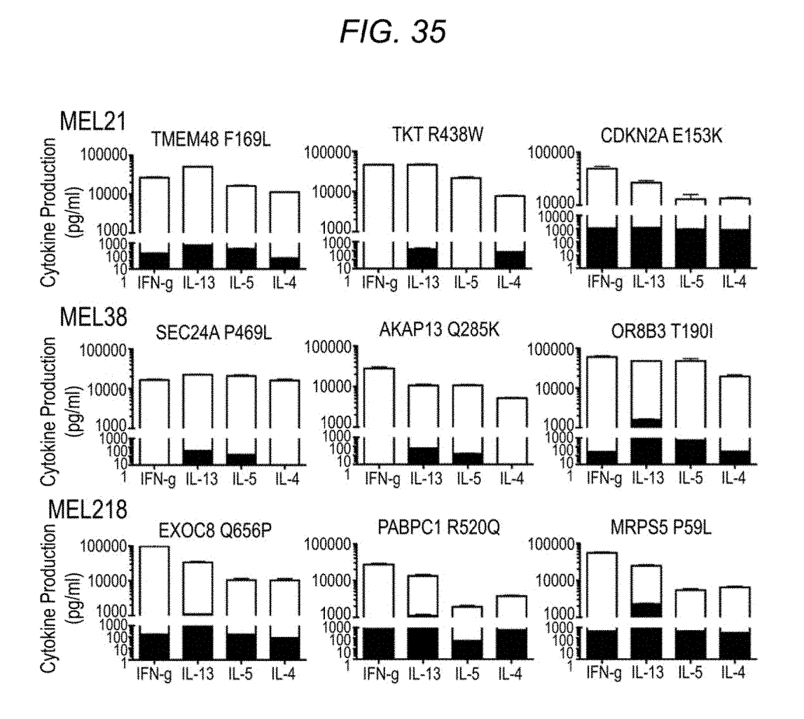

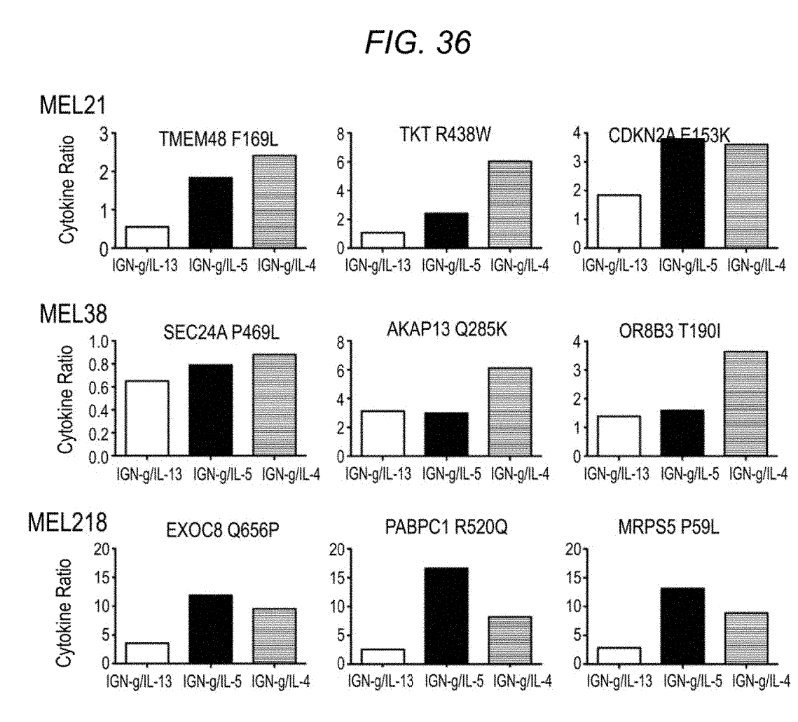

The cytokine production profile of these cells was characterized as previously described (Carreno, B. M., et al., J. Clin. Invest., 123, 3383-3394, 2013; Fridman, W. H., et al., Nat. Rev. Cancer, 12, 298-306, 2012). This characterization is illustrated in FIG. 35: Neoantigen-specific T cells were stimulated with artificial antigen presenting cells in the presence (open bar) or absence (close bar) of AAS-peptide and supernatants were harvested at 24 h. Cytokine production was determined using MILLIPLEX® MAP Human Cytokine Panel I. Mean values+/−SD of duplicates are shown. Cytokine production by T cells in the absence of any stimuli was <100 pg/mL. A representative experiment of 2 performed is shown. FIG. 36 illustrates a comparison of production of Type 1 (IFN-γ) and Type 2 (IL-4, IL-5, IL-13) cytokines among neoantigen-specific T cells, a cytokine index was derived by dividing pg/mL IFN-γ by pg/mL IL-13, IL-5 or IL-4. IFN-γ/IL-13, IFN-γ/IL-5 and IFN-γ/IL-4 ratios above 1 are indicative of Type 1 phenotype. Results are representative of two experiments. Upon antigen stimulation, most vaccine-induced neoantigen-specific T cells produced high amounts of IFN-γ relative to IL-4, IL-5 and IL-13, a pattern that is indicative of a type 1 phenotype (FIG. 35-36). However, SEC24A P469L specific cells exhibited a type 2-skewed phenotype (high IL-4, IL-5 and IL-13 levels relative, to IFN-γ), and TMEM48 F169L specific T cells showed a mixed phenotype with only higher IL-13 (hut not IL-4 or IL-5) levels relative to IFN-γ (FIG. 35-36).

### Example 13

This example illustrates the in vitro detection of neoantigens that are presented to immune cells in vivo.

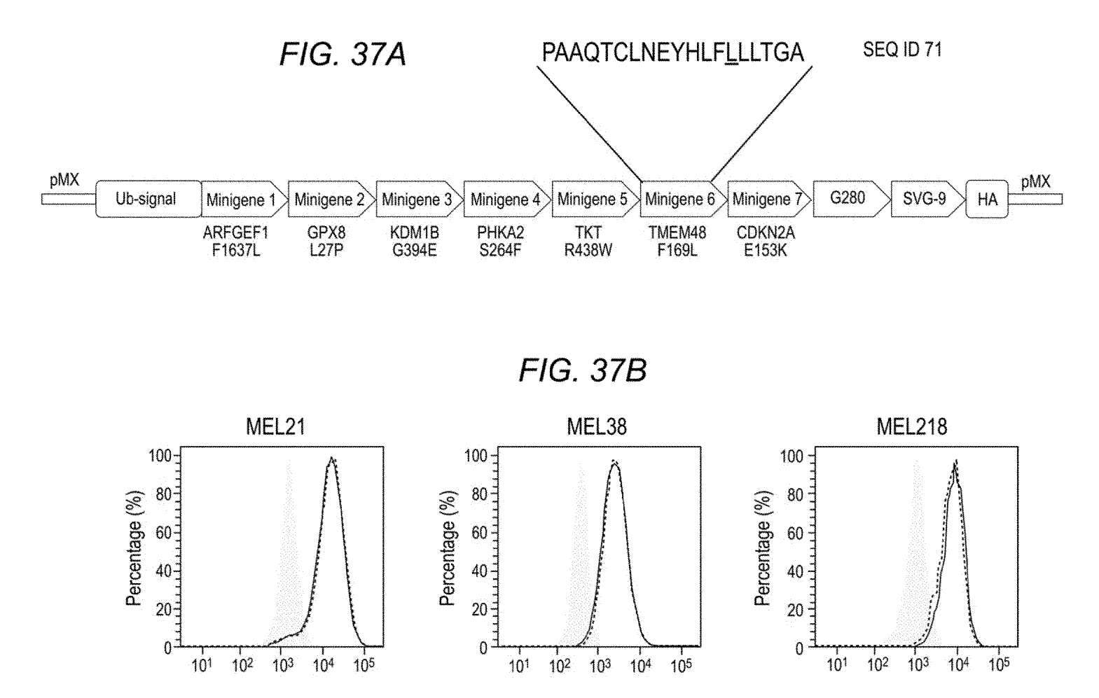

Tandem mini-gene constructs (TMC) were used for evaluating processing and presentation of neoantigens. The structure of a representative TMC (MEL21 AAS sequences) is shown in FIG. 37A. All constructs were 19-21-mers encoding AAS- or WT-sequences for peptides included in vaccine. No spacers are present between sequences. A ubiquitination signal and two mini-gene controls (encoding G280 and WNV SVG9 peptides) were included to monitor processing and presentation. The amino acid sequence of a 21-mer encoding TMEM48 F169L is shown with mutated amino acid residue underlined. TMC also encoded the West Nile Virus (WNV) SVG9 (McMurtrey, C. P., et al., P.N.A.S., 105, 2981-2986, 2008) and melanoma G280 (Cox, A. L., et al., Science, 264, 716-719, 1994) antigenic determinants as controls (Table 6).

TMC were cloned into pMX (GFP+), expressed as retrovirus and used to transfect the HLA-A*02:01+ melanoma lines DM6 (Darrow, T. L., et al., J. Immunol., 142, 3329-3335, 1989) or A375 (obtained from ATCC and mycoplasma free). TMC expressing cells were selected by sorting for GFP+ cells expressing cell surface HLA-A*02:01/SVG9 peptide complexes as detected by a T cell receptor mimic (TCRm) monoclonal antibody (Kim S., et al., J. Immunol., 184, 4423-4430, 2010). AAS- and WT-TMC reactivity with the HLA-A*02:01/SVG9 peptide complex specific TCRm monoclonal antibody validated expression of the mini-gene constructs. FIG. 37B demonstrates that expression of AAS- and WT-TMC constructs was determined using a TCR-mimic monoclonal antibody that detects HLAA*02:01/SVG9 (SVGGVFTSV SEQ ID NO: 31) complexes Kim S., et al., J. Immunol., 184, 4423-4430, 2010). Results are shown for parental DM6 (shaded histogram) and DM6 cells expressing AAS—(dashed line) and WT (solid line) TMC constructs. A representative experiment of four performed is shown.

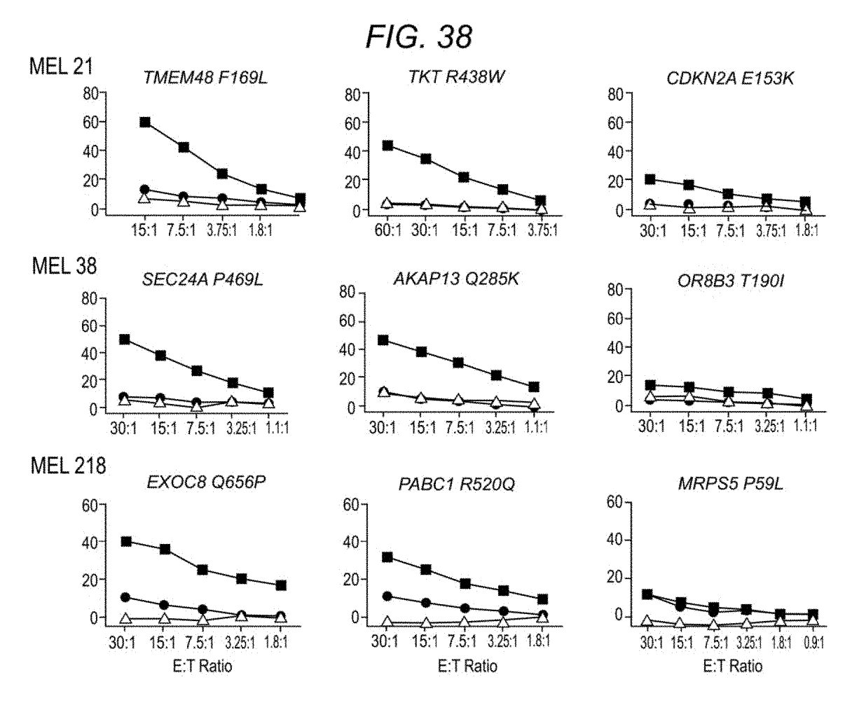

DM6 cells expressing TMC were labeled with 25μCi ⁵¹Cr for 1 h, washed and tested as targets in a standard 4 h assay using neoantigen-specific T cells as effectors (Carreno B. M. et al. 2012 J Immunol 188, 5839). DM6 cells expressing AAS—(closed rectangles) or WT—(closed circles) TMC were co-cultured with neoantigen-specific T cells at a 1:1 ratio, supernatants harvest at 16 h and IFN-γ production evaluated by ELISA as described (Carreno, B. M., et al., J Immunol., 188, 58395849, 2014; plots in FIG. 38). Open triangles represent lysis obtained with parental DM6 cells. Percent specific lysis of triplicates (mean+standard deviation) is shown for each E:T ratio; spontaneous lysis was <5%. A representative experiment of two independent evaluations is shown.

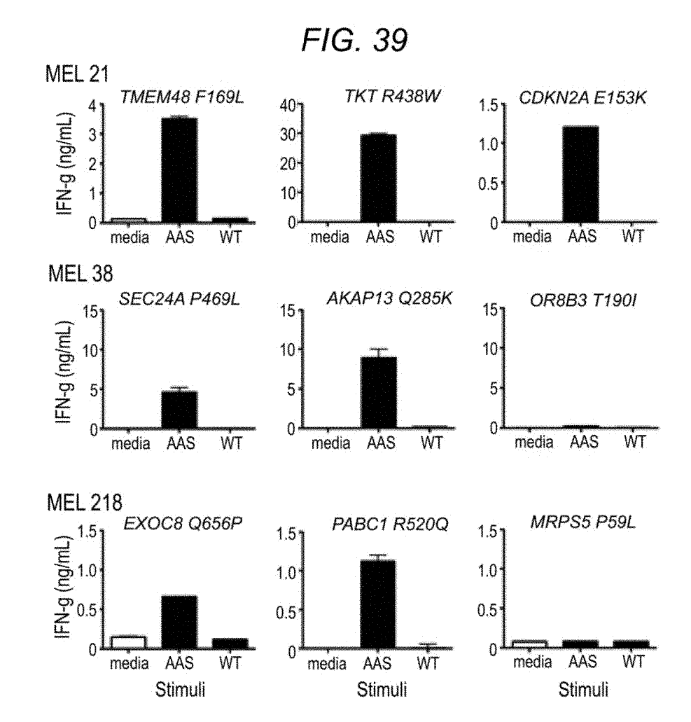

FIG. 39 illustrates that neoantigen-specific CD8 T cells were co-cultured with DM6 expressing AAS- or WT-encoding TMC for 20 h and IFN-γ production determined by ELISA. T cells cultured with parental DM6 cells are indicated as media. Mean values+/−SD of duplicates are shown. Results are representative of 2 experiments performed. Seven (TMEM48 F169L, TKT R438W, CDKN2A E153K, SEC24A P469L, AKAP13 Q285K, EXOC8 Q656P and PABPC1 R520Q) of the nine immunogenic neoantigens are processed and presented as evidenced by cytotoxic activity (FIG. 38) and IFN-γ production (FIG. 39) by corresponding neoantigen-specific T cells upon co-culture with DM6 expressing AAS-encoding TMC. In contrast, neither cytotoxic activity (FIG. 38) nor IFN-γ production (FIG. 39) was observed upon co-culture of OR8B3 T190I- and MRPS5 P59L-specific T cells with DM6 expressing AAS-encoding TMC showing that these neoantigens are not processed and presented from endogenously expressed protein. None of the neoantigen-specific T cells recognized WT-encoding TMC (FIGS. 38 and 39). Based on these findings and the immune monitoring results (FIG. 31), the nine neoantigens identified in this study fall into three distinct antigenic determinant categories (Sercarz, E. E., et al., Annu. Rev. Imunol., 11, 729-766, 1993; Assarsson, E., et al., J. Immunol., 178, 7890-7901, 2007). TMEM48 F169L, SEC24A P469L, and EXOC8 Q656P represent dominant antigens as T cell immunity was detected prior to vaccination (naturally occurring) (FIG. 31 and these neoantigens are processed and presented from endogenously expressed protein (FIG. 38). TKT R438W, CDKN2A E153K, AKAP13 Q285K and PABPC1 R520Q are characterized as subdominant antigens as T cell immunity required peptide vaccination (FIG. 31) and these neoantigens are processed and presented from endogenously expressed protein (FIG. 38). And finally, OR8B3 T190I and MRPS5 P59L constitute cryptic antigens since peptide vaccination elicited T cell immunity but these neoantigens are not processed from endogenously expressed protein.

### Example 14

This example illustrates the use of proteomic techniques to determine which neoantigens are presented to cells in vivo.

To validate neoantigen processing and presentation, proteomic analysis was performed on peptides eluted from soluble HLA-A*02:01 molecules isolated from melanoma cells expressing a TMC encoding AAS candidates from patient MEL218 tumor (Sercarz, E. E., et al., Annu. Rev. Imunol., 11, 729-766, 1993; Assarsson, E., et al., J. Immunol., 178, 7890-7901, 2007). TMC expressing A375 melanoma cells were transfected with soluble HLA-A*02:01(sHLA-A*02:01) and single cell sorted for a high (>1000 ng/ml in static culture) sHLAA*02:01 producing clone. The sHLA-A*02:01 construct includes a C-terminal VLDLr epitope purification tag (SVVSTDDDLA SEQ ID NO. 32) that is recognized by the anti-VLDLr mAb (ATCC CRL-2197). This antibody was also used for quantification of sHLA production as the capture antibody in a sandwich ELISA, with an antibody directed against β2-microglobulin (Dako Cytomation) as the detector antibody. Cells were grown in roller bottles and sHLA/peptide complexes were purified from supernatants by affinity chromatography with the anti-VLDLr antibody (Kaabinejadian, S., et al., P.L.o.S. One, 8, e66298, 2013). Eluate fractions containing sHLA/peptide complexes were brought to a final acetic acid concentration of 10%, pooled, and heated to 78° C. in a water bath. Peptides were purified through a 3 kDa molecular weight cutoff cellulose membrane (EMD Millipore) and lyophilized.

Synthetic peptides corresponding to the mutant sequences were resuspended in 10% acetic acid in water at 1 μM, and fractionated by RP-HPLC with an acetonitrile gradient in 10 mM ammonium formate at pH 10. Peptide-containing fractions were dried and resuspended in 25 ul of 10% acetic acid and subjected to nanoscale RP-HPLC at pH 2.5 utilizing an Eksigent nanoLC coupled to a TripleTOF 5600 (AB Sciex) quadrupole time-of-flight mass spectrometer (LC/MS). Information dependent acquisition (IDA) was used to obtain MS and MS/MS fragment spectra for peptide ions. The sequence of each peptide was determined by observed mass and fragment ions, and the 1st dimension fraction number and LC/MS retention times were recorded.

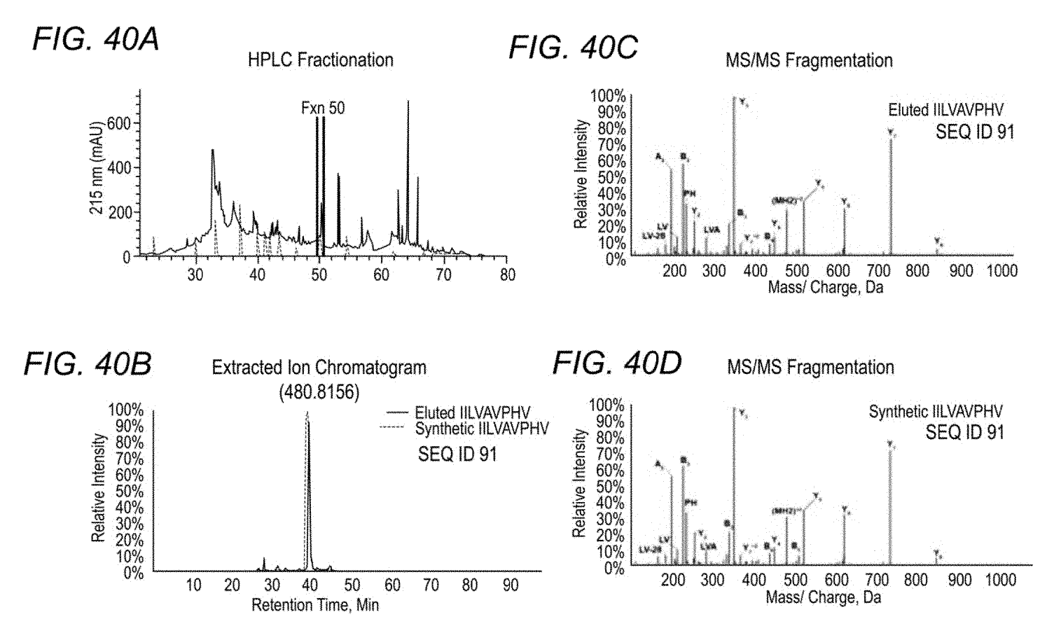

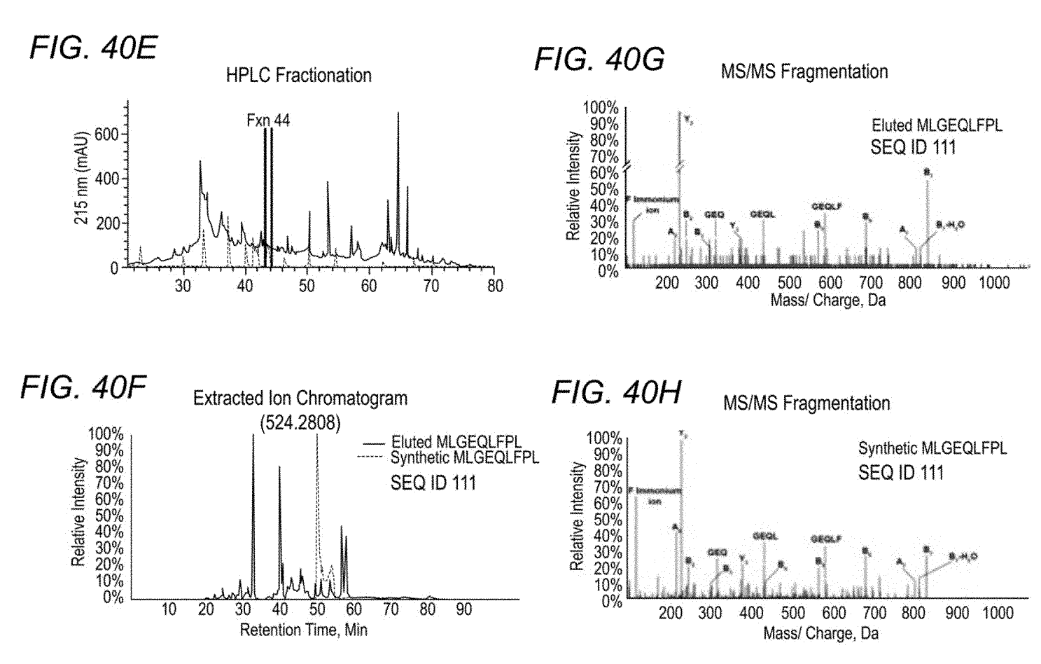

Next, peptides purified from TMC expressing A375 melanoma cells were resuspended in 10% acetic acid and HPLC fractionated under the same conditions and gradient method. Reverse phase HPLC was used to reduce the complexity and determine the elution profile of the pool of soluble HLA-A*02:01 restricted peptides presented by melanoma cells, as well as, the synthetic AAS peptide mixture. FIGS. 40A and 40E illustrate RP-HPLC fractionation of HLA-A*02:01 peptides eluted from the AAS-TMC expressing melanoma cell line (solid trace) and the synthetic peptide mixture containing MEL218 neoantigen candidates (dashed trace), with fraction 50 (FIG. 40A) and fraction 44 (FIG. 40E) indicated. The HPLC fractions corresponding to those containing the synthetic peptides were then subjected to the same LC/MS conditions. Resulting spectra were found positive for the presence of the mutant peptides if the following criteria were met: 1. The observed fragment ions were in the same RP-HPLC fraction as the synthetic, 2. LC/MS elution time was within 2 minutes of the synthetic, and 3. Fragment ion masses matched those of the synthetic with an accuracy of ±25 ppm. PEAKVIEW® Software version: 1.2,0,3 was used for exploring and interpreting of the LC/MS data.

Separation and sequencing of peptides were carried out by two-dimensional liquid chromatography, followed by information dependent acquisition (IDA) generated tandem MS (MS/MS). For the first dimension, the peptide sample was loaded on a reverse-phase C¹⁸ column (pore size, 110 Å; particle size, 5 μm; 2 mm i.d. by 150 mm long Gemini column; Phenomenex) with a Michrom BioResources Paradigm MG4 high performance liquid chromatograph (HPLC) with UV detection at 215 nm wavelength. Elution was at pH 10 using 10 mM ammonium formate in 2% acetonitrile/98% water as solvent A and 10 mM ammonium formate in 95% acetonitrile/5% water for solvent B. The 1st dimension HPLC column was preequilibrated at 2% solvent B, then the peptide sample, dissolved in 10% acetic acid/water, was loaded at a flow rate of ˜120 μl/min over an 18 minute period. Then a two segment gradient was performed at 160 μl/min; the 1st segment was a 40 minute linear gradient from 4% B to 40% B, followed by an eight minute linear gradient from 40% B to 80% B. Forty peptide-rich fractions were collected and dried by vacuum centrifugation.

For the second dimension chromatography, each dried fraction was resuspended in 10% acetic acid and subjected to nano-scale RP-HPLC (Eksigent nanoLC415, AB Sciex). The second dimension nano-HPLC setup included a C¹⁸ trap column (350 μm i.d. by 0.5 mm long; ChromXP (Eksigent) with 3 μm particles and 120 Å pores and a ChromXP, C¹⁸ separation column with dimensions of 75 μm i.d. by 15 cm long packed with the same medium. A two-solvent system was utilized, where solvent A is 0.1% formic acid in water and solvent B contains 0.1% formic acid in 95% acetonitrile/5% water. Samples were loaded at 5 μL/min flow rate on the trap column and at 300 nL/min flow rate on the separation column that was equilibrated in 2% solvent B. The separation was performed by a program with two linear gradients: 10% to 40% solvent B for 70 min and then 40% to 80% solvent B for 7 min. The column effluent was connected to the nanospray III ion source of an AB Sciex TripleTOF 5600 quadrupole-time of flight mass spectrometer with the source voltage set to 2400 v.

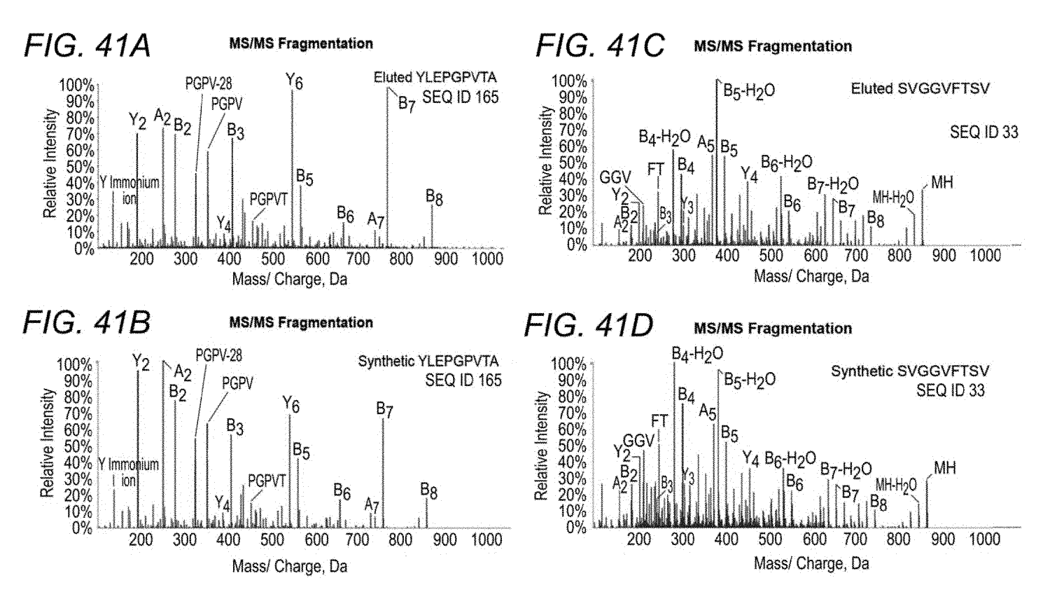

Extracted ion chromatograms revealed the presence of an eluted peptide with a retention time within 2 minutes of synthetic EXOC8 Q656P peptide in fraction 50. FIG. 40B illustrates an extracted ion chromatogram of the parent ion with the theoretical m/z of 480.8156 (+2) in HPLC fraction 50 from the HLA-A*02:01 eluted peptides (solid line) overlaid with the EXOC8 Q656P synthetic peptide (dashed line). MS/MS fragmentation pattern comparison of the eluted and the synthetic peptides ensured EXOC8 Q656P sequence identity and confirmed HLA-A*02:01 presentation of this dominant neoantigen. The eluted EXOC8 Q656P peptide MS/MS fragmentation pattern is illustrated in FIG. 40C and that of the corresponding synthetic peptide is illustrated in FIG. 40D. A similar analysis of fraction 44 demonstrated the HLA-A*02:01 presentation of subdominant neoantigen PABPC1 R520Q. FIG. 40F illustrates the extracted ion chromatogram of the parent ion (depicted in FIG. 40E, supra) with the theoretical m/z 524.2808 (+2) in HPLC fraction 44 from the HLA-A*02:01 elated peptides (solid line) overlaid with the PABPC1 R520Q synthetic peptide (dashed line). The MS/MS fragmentation pattern of the eluted peptide is shown in FIG. 40G and that of the corresponding synthetic peptide is shown in 3H. Altogether, these results show that two of the 7 neoantigens included inpatient MEL218 vaccine, along with antigen controls WNV SVG9 and G280, are processed and presented in the context of HLA-A*02:01 molecules. MS/MS fragmentation pattern of the peptide elated from HLA-A*02:01 identified as YLEPGPVTA (SEQ ID No. 165) (FIG. 41A), and the corresponding G280 synthetic peptide. MS/MS fragmentation pattern (FIG. 41C) of the peptide elated from HLA-A*02:01 identified as SVGGVFTSV (SEQ ID No. 33) (FIG. 41B), and the corresponding WNV SVG9 synthetic peptide (FIG. 41D).

### Example 15

This example illustrates characterization of the composition and diversity of neoantigen-specific T cells and the effect vaccination can have on these repertoires.

Short-term ex-vivo expanded neoantigen-specific T cells were purified to 97-99% purity by cell sorting in a Sony SY3200 BSC (Sony Biotechnology) fitted with a 100 um nozzle, at 30 psi, using 561 nm (585/40) and 642 nm (665/30) lasers and cell pellets were prepared. DNA isolation and TCRβ sequencing was performed by Adaptive Biotechnologies and The Genome Institute at Washington University. Sequencing was performed at either survey (for neoantigen-specific TCRβ reference libraries) or deep (for pre- and post-vaccine CD8+ T cell populations) level (Robins, H., et al., J. Immunol. Methods, 375, 14-19, 2012; Carlson, C. S., et al., Nat. Commun., 4, 2680, 2013). TCRβ V-, D-, J-genes of each CDR3 regions were defined using IMGT (ImMunoGeneTics)/Junctional algorithms and data uploaded into the ImmunoSeq Analyzer (Adaptive Biotechnologies) for analysis. Complete amino acid identity between the reference library and pre- and post-vaccine CD8 samples was required for assigning a TCRβ match. In the reference library, TCRβ clonotypes with frequencies above 0.1% (>100-fold sequencing depth) were set as a threshold for identification of neoantigen-specific TCRβ CDR3 sequences within pre- and post-vaccine CD8+ T cell populations.

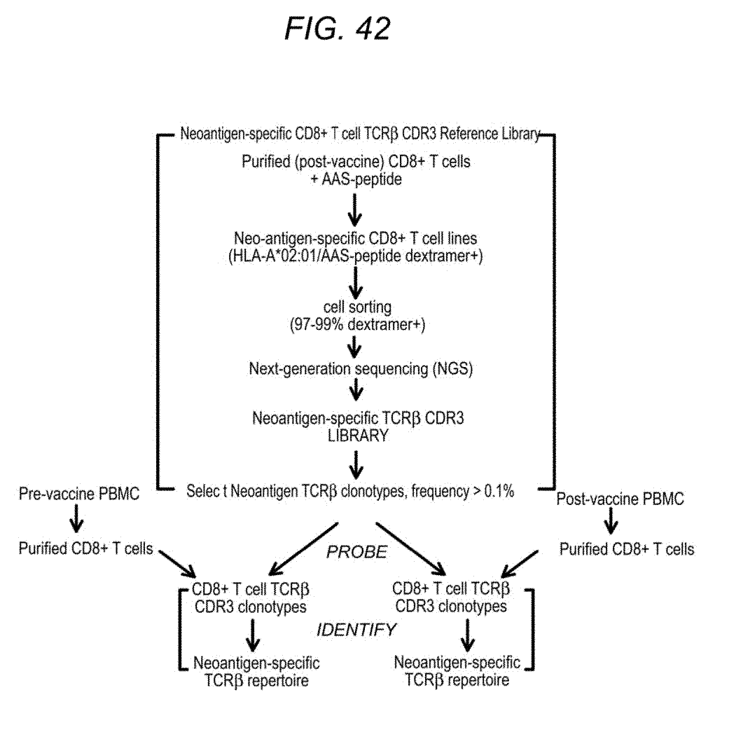

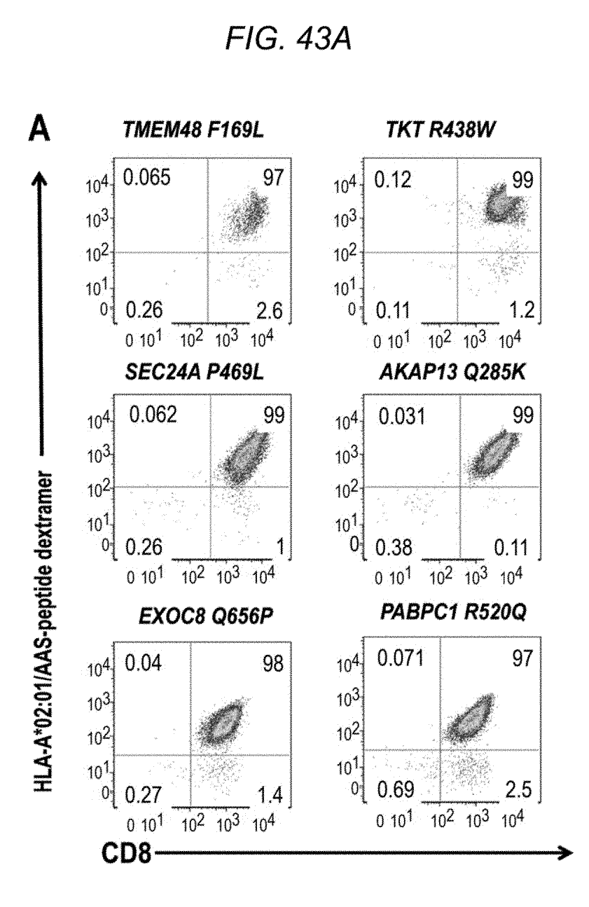

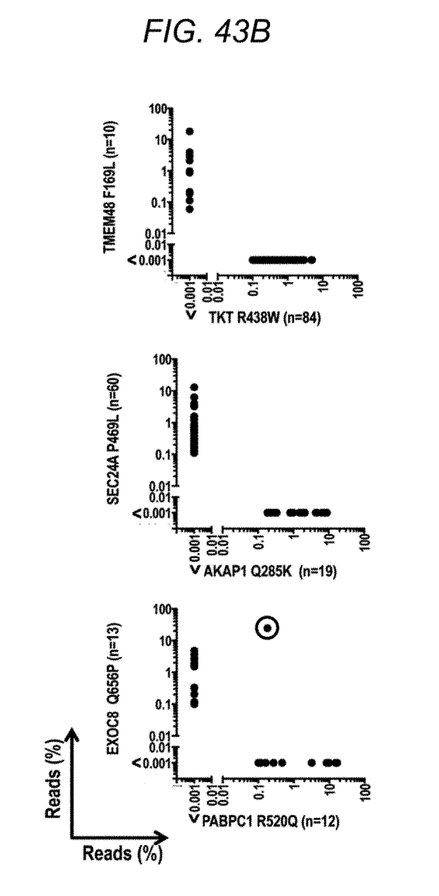

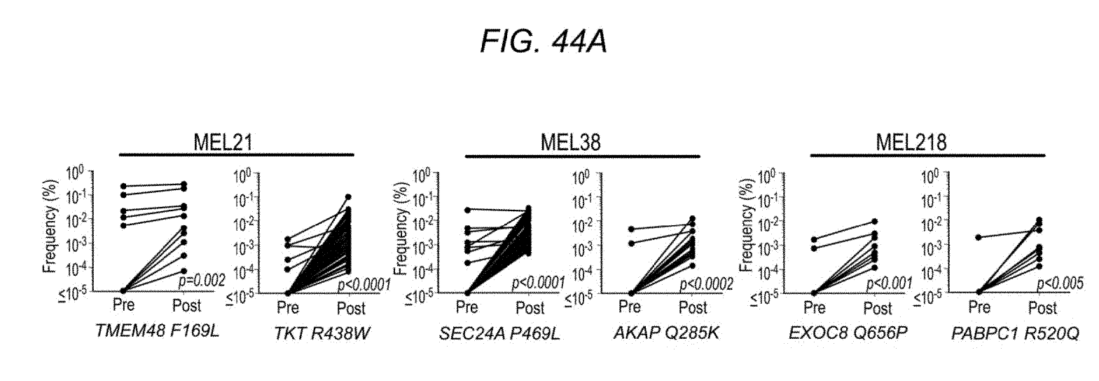

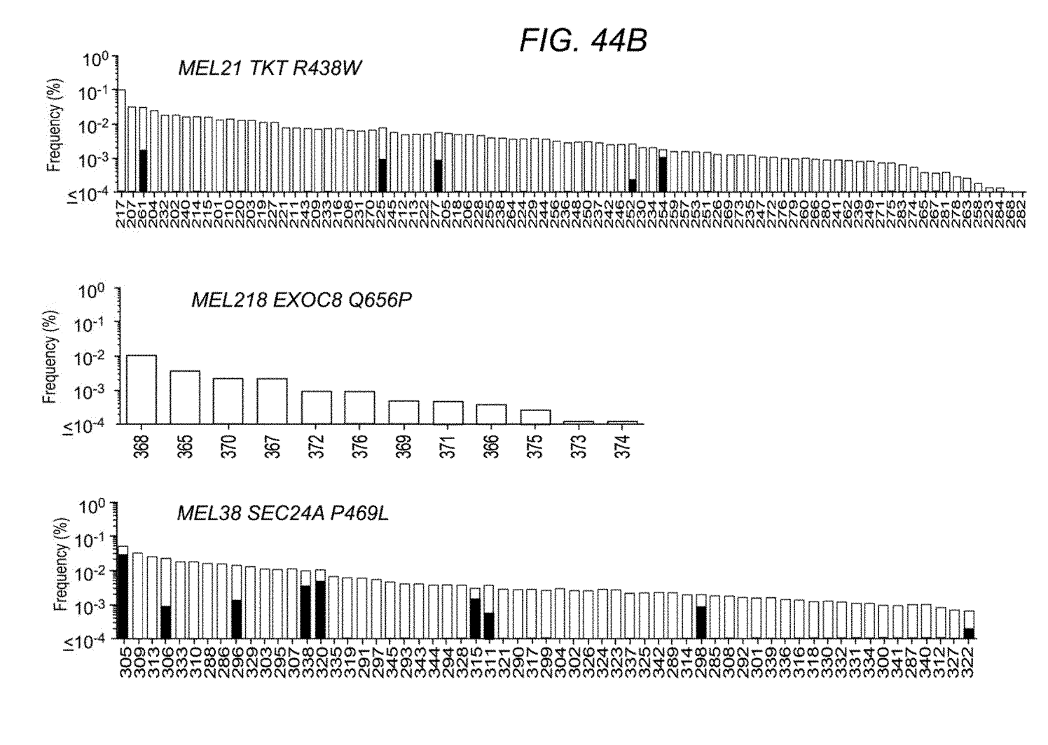

Reference T cell receptor-β (TCRβ) complementarity-determining region 3 (CDR3) sequence libraries (shown schematically in FIG. 42, Tables 7-11) were generated from short-term expanded sorted neoantigen-specific T cells (97-99% dextramer-positive), in Tables 7-11, TCRBV, TCRBD and TCRBJ are shown according to consensus nomenclature and CDR3 sequence for each clonotype indicated. Read counts indicates the number of times a given CDR3 sequence was found in the short term ex-vivo expanded neoantigen population. TCRβ clonotypes with frequencies above 0.1% (>100-fold sequencing depth), in reference library, were set as a threshold for identification of neoantigenspecific TCRβ CDR3 sequences within CD8+ T cell populations isolated from PBMC obtained pre- and post-vaccination. FIG. 43A illustrates profiles purified neoantigen-specific CD8+ T cells used for the generation of TCRβ CDR3 reference libraries. In FIG. 43A, purified CD8+ T cells isolated from PBMC obtained after vaccination were stimulated in an antigen-specific manner as described supra. Cells were stained using HLA-A*02:01/AAS-peptide dextramers and anti-CD8 monoclonal antibody; neoantigen-specific CD8+ cells were sorted in a Sony SY3200 BSC Cell Sorter. Purity of post-sort populations is shown in dot plots (upper right quadrants, 97-99% purity). FIG. 43B illustrates the comparison of clonotype distribution in sorted/expanded dominant and subdominant neoantigen-specific CD8 T cells obtained from each of the indicated patients. These clonotypes represent the TCRβ CDR3 reference libraries used for probing pre- and post-vaccine CD8+ T cell populations. Frequencies are shown as percent of total reads. Reference library comprised clonotypes with frequencies of 0.1 or above (Lossius, A., et al., Eur. J. Immunol., 44, 3439-3452, 2014). The total number of clonotypes in each antigen population is indicated in the x- and y-axis and CDR3 sequences arc listed in Tables 7-11. The one clonotype that overlapped between EXOC8 Q656P and PABPC1 R520Q (indicated by circle) was excluded from analysis. These sequence libraries were used to characterize neoantigen TCRβ clonotypes in purified CD8+ T cells isolated from pre- and post-vaccine PBMC samples (Robins, et al., J. Immunol Methods, 375, 14-19, 2012; Lossius, A., et al., Eur. J. Immunol., 44, 3439-3452, 2014; Robins, H. S., et al., Sci. Transl. Med., 5, 214ra169, 2013). In pre-vaccination CD8+ T cell populations, as few as one and as many as 10 unique TCRβ clonotypes per neoantigen were identified. FIG. 44A summarizes the TCRβ clonotypes identified, using neoantigen-specific TCRβ CDR3 reference libraries (see Tables 7-11), in CD8+ T cell populations isolated from PBMC obtained before and after vaccination. Each symbol represents a unique TCRβ sequence and its frequency (%) in pre- and post-vaccine samples. Wilcoxon-signed rank test was performed and p values indicated in figure. Thus, vaccination increased the frequency of most existing pre-vaccine TCRβ clonotypes and revealed new clonotypes for all 6 neoantigens (FIG. 44A). For both dominant and subdominant neoantigens, the TCRβ repertoire was increased significantly after vaccination. FIG. 44B illustrates TCRβ CDR3 sequence of clonotypes (Tables 7-11) identified in pre—(black bars) and post—(white bars) vaccine CD8+ T cell populations for neoantigens TKT R438W (pre=5, post=84 clonotypes); SEC24A P469L (pre=9, post=61) and EXOC8 Q656P (pre=2, post=12). Frequency of each unique clonotype is reported as percentage of total read counts. 84 clonotypes representing TCRβ families are detected for TKT R438W, 61 clonotypes representing 12 TCRβ families are detected for SEC:24A P469L and 12 clonotypes representing 8 TCRβ families are detected for EXOC8 Q656P (FIG. 44B). Thus, peptide vaccination with functionally mature DC can promote the expansion of a highly diverse neoantigen TCR repertoire.

### Example 16

This example illustrates vaccination of patients using multiple HLA cell types.

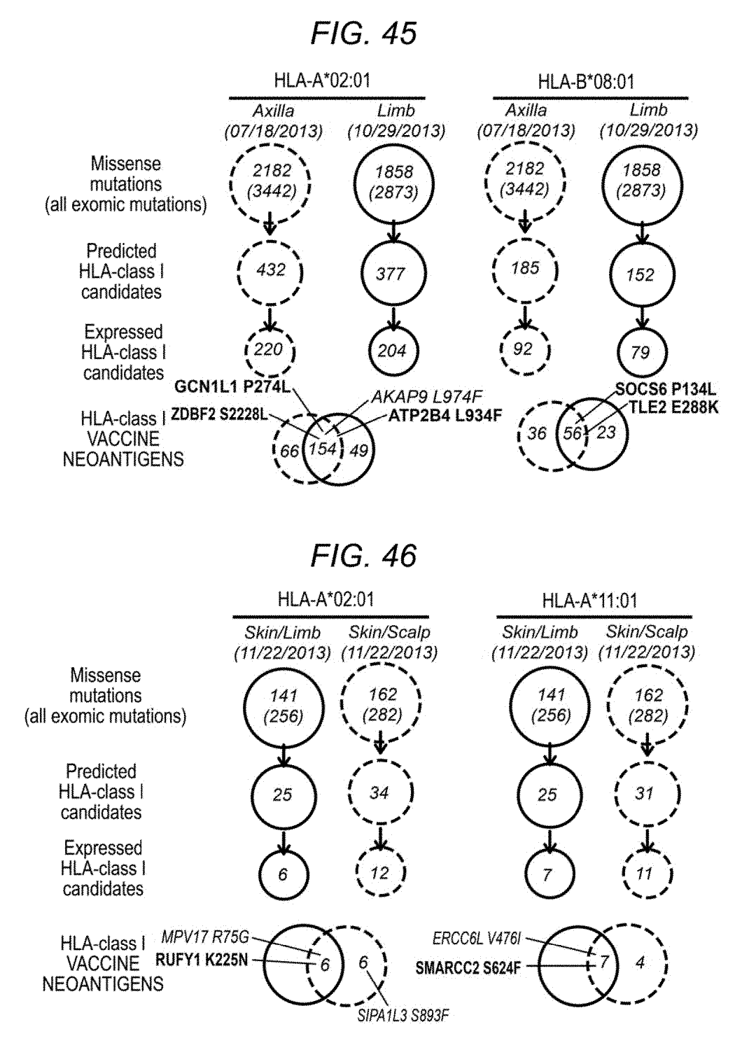

Distribution of somatic (exomic and missense) mutations was identified in metachronous tumors of patients MEL66 is illustrated in FIG. 45 (anatomical location and date of collection indicated). HLA-A*02:01- and HLA-B*08:01-binding candidate peptides were identified in silico according to the methods of the present teachings among amino acid substitutions present in the patient's tumor; expression of genes encoding mutated proteins was determined from cDNA capture data. Venn diagrams show expression, among metachronous tumors, of mutated genes encoding vaccine neoantigens. The identities of the 6 immunogenic neoantigens identified among the 10 included in vaccine are indicated; type style identifies naturally occurring (italics) and vaccine-induced (bold) neoantigens.

Distribution of somatic (exomic and missense) mutations identified in metachronous tumors of patients MEL69 is illustrated in FIG. 46 (anatomical location and date of collection indicated). HLA-A*02:01- and HLA-A*11:01-binding candidate peptides were identified in among amino acid substitutions in the patient's tumor according to a method of the present teachings; expression of genes encoding mutated proteins was determined from cDNA capture data (Table 12). Venn diagrams show expression, among metachronous tumors, of mutated genes encoding vaccine neoantigens. The identities of the 5 immunogenic neoantigens identified among the 10 included in vaccine are indicated; type style identifies naturally occurring (italics) and vaccine-induced (bold) neoantigens.

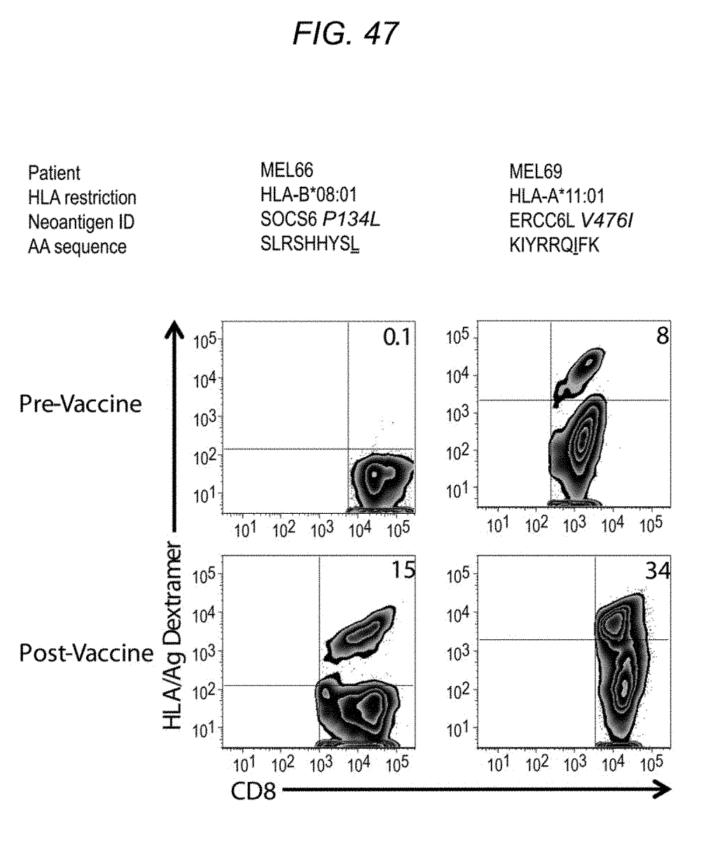

The vaccine for patient MEL66 included neoantigens that bound to HLA-A*02:01 and HLA-B*08:01 molecules. The vaccine for MEL69 included neoantigens that bound to HLA-A*03:01 and HLA-A*11:01 molecules. Both vaccines were prepared by contacting the neoantigens with the patient's own dendritic cells and maturing them prior to administration in accordance with the present teachings. Representative results (dextramer assay) to neoantigens restricted to these alleles are shown (FIG. 47) before DC vaccination (pre-vaccine) and at peak of immune response (post-vaccine). Numbers within dot plots represent percentage neoantigen-specific T cells within the lymph+/CD8+ gated cells. A naturally occurring response to HLA-A*11:01-restricted neoantigen ERCC6L V476I was observed in patient MEL69.

All cited publications are hereby incorporated by reference, each in its entirety.

TABLE 1
Analysis of missense mutations by prediction algorithms for binding to HLA-A*0201

| | | Protein | MUTATED | | | | WILD-TYPE | | | | |
|---|---|---|---|---|---|---|---|---|---|---|---|
| | | AA | | Sequence | Binding | Affinity | | Sequence | Binding | Affinity | |
| CHR | Gene | Change | AA seq | Listing | (nM) | (h) | AA seq | Listing | (nM) | (h) | |
| 15 | AKAP13 | Q285K | KLMNIQQKL | SEQ ID | 19 | 5.02 | KLMNIQQQL | SEQ ID | 17 | 4.72 | |
| | | | | NO: 1 | | | | NO: 16 | | | |
| 8 | ARFGEF1 | R782C | FVSALCMFL | SEQ ID | 19 | 3.09 | FVSALRMFL | SEQ ID | 88 | 0.88 | |
| | | | | NO: 2 | | | | NO: 17 | | | |
| 17 | CCDC57 | R353C | QLCEDASTV | SEQ ID | 352 | 2.77 | QLREDASTV | SEQ ID | 2265 | 1.02 | |
| | | | | NO: 3 | | | | NO: 18 | | | |
| 8 | CPNE3 | P448L | LMSIIIVGV | SEQ ID | 16 | 6.98 | PMSIIIVGV | SEQ ID | 817 | 1.77 | |
| | | | | NO: 4 | | | | NO: 19 | | | |
| 14 | DICER1 | Y153C | LIMTCCVAL | SEQ ID | 46 | 4.99 | LIMTCYVAL | SEQ ID | 43 | 1.88 | |
| | | | | NO: 5 | | | | NO: 20 | | | |
| 16 | GLYR1 | P386L | ALVSGNQQL | SEQ ID | 273 | 1.05 | APVSGNQQL | SEQ ID | 25384 | 0.3 | |
| | | | | NO: 6 | | | | NO: 21 | | | |
| 1 | HSD17B7 | H108Y | YISKCWDYA | SEQ ID | 233 | 0.94 | YISKCWDHA | SEQ ID | 971 | 0.78 | |
| | | | | NO: 7 | | | | NO: 22 | | | |
| 22 | IL17RA | T362M | FIMGISILL | SEQ ID | 4 | 7.46 | FITGISILL | SEQ ID | 24 | 3.58 | |
| | | | | NO: 8 | | | | NO: 23 | | | |
| 1 | KIF14 | G842W | IQLSWVLIA | SEQ ID | 144 | 0.7 | IQLSGVLIA | SEQ ID | 658 | 0.59 | |
| | | | | NO: 9 | | | | NO: 24 | | | |
| 12 | MED13L | G2045W | ILMTWNLHS | SEQ ID | 259 | 0.97 | ILMTGNLHS | SEQ ID | 1243 | 0.78 | |
| | | | | NO: 10 | | | | NO: 25 | | | |
| 1 | OR5K2 | G64E | YIFLENLAL | SEQ ID | 55 | 1.15 | YIFLGNLAL | SEQ ID | 38 | 1.02 | |
| | | | | NO: 11 | | | | NO: 26 | | | |
| 11 | OR8B3 | T190I | QLSCISTYV | SEQ ID | 18 | 6.54 | QLSCTSTYV | SEQ ID | 35 | 5.06 | |
| | | | | NO: 12 | | | | NO: 27 | | | |
| 5 | SEC24A | P469L | FLYNLLTRV | SEQ ID | 4 | 19.62 | FLYNPLTRV | SEQ ID | 6 | 13.57 | |
| | | | | NO: 13 | | | | NO: 28 | | | |
| 17 | TAOK1 | A196V | WMAPEVILV | SEQ ID | 7 | 4.32 | WMAPEVILA | SEQ ID | 40 | 1.32 | |
| | | | | NO: 14 | | | | NO: 29 | | | |
| 6 | UTRN | Q1058K | QLDKCSAFV | SEQ ID | 12 | 6.63 | QLDQCSAFV | SEQ ID | 22 | 7.65 | |
| | | | | NO: 15 | | | | NO: 30 | | | |
| | | | | | | | | | abdominal wall | | |
| | | | breast (Feb. 14, 2013) | | | | | | (Apr. 16, 2013) | | |
| | | Protein | | Transcrip- | | | | Protein | | Transcrip- | |
| | | AA | Exome | tome Var | | | | AA | Exome | tome Var | |
| CHR | Gene | Change | Var Freq | Freq | FPKM | CHR | Gene | Change | Var Freq | Freq | FPKM |
| 15 | AKAP13 | Q285K | 13.97 | 23.49 | NR | 15 | AKAP13 | Q285K | 25.13 | 26 | NR |
| 8 | ARFGEF1 | R782c | 19.17 | 15.07 | 23.73 | 8 | ARFGEF1 | R782C | 11.65 | 10.79 | 17.51 |
| 17 | CCDC57 | R353C | 23.97 | 30.23 | 0.79 | 17 | CCDC57 | R353C | | | |
| 8 | CPNE3 | P448L | 15.49 | 17.46 | 0.29 | 8 | CPNE3 | P448L | 16.11 | 16.87 | 2.27 |
| 14 | DICER1 | Y153C | 39.34 | 49.55 | 7.21 | 14 | DICER1 | Y153C | 31.03 | 31.48 | 8.05 |
| 16 | GLYR1 | P386L | 48.64 | 42.81 | 35.963 | 16 | GLYR1 | P386L | 43.18 | 38.21 | 32.52 |
| 1 | HSD17B7 | H108Y | 17.89 | 19.97 | 0.11 | 1 | HSD17B7 | H108Y | 18.41 | 17.86 | 0.2 |
| 22 | IL17RA | T362M | 30.97 | 26.83 | 0.22 | 22 | IL17RA | T362M | | | |
| 1 | KIF14 | G842W | 20.97 | 22.92 | 3.63 | 1 | KIF14 | G842W | 16.27 | 22.22 | 2.1 |
| 12 | MED13L | G2045W | 44.44 | 43.58 | 13.64 | 12 | MED13L | G2045W | 30.43 | 28.1 | 14.97 |
| 1 | OR5K2 | G64E | 29.67 | 63.64 | 0.47 | 1 | OR5K2 | G64E | | | |
| 11 | OR8B3 | T190I | 60.52 | NR | NR | 11 | 048B3 | T190I | 20.23 | NR | NR |
| 5 | SEC24A | P469L | 37.5 | 42.48 | 1.34 | 5 | SEC24A | P469L | 24.05 | 20.12 | 0.39 |
| 17 | TAOK1 | A196V | 30.8 | 35.31 | 11.32 | 17 | TAOK1 | A196V | 31.57 | 29 | 8.28 |
| 6 | UTRN | Q1058K | 58.33 | 81.5 | 15.94 | 6 | UTRN | Q1058K | 38.98 | 57.43 | 12.56 |

TABLE 2
MEL21

| | | | | | | Predicted Affinity(nM)^{a} | | |
|---|---|---|---|---|---|---|---|---|
| | Hugo | | | wild-type | | | wild- | Amino Acid |
| CHR | Symbol | AAS- peptide | AAS-SEQID | peptide | WT SEQ ID | mutated | tpe | Substitution (AAS) |
| 1 | AGMAT | NLSGNTALL | SEQ ID. 35 | DLSGNTALL | SEQ ID. 36 | 247 | 8129 | D326N |
| 8 | ARFGEF1 | QTIDNIVFL | SEQ ID. 37 | QTIDNIVFF | SEQ ID. 38 | 387 | 10867 | F1637L |
| 9 | CDKN2A | KMIGNHLWV | SEQ ID. 39 | EMIGNHLWV | SEQ ID. 40 | 14 | 1044 | E153K |
| 19 | CYP2S1 | FTMLALQDL | SEQ ID. 41 | FTMLALRDL | SEQ ID. 42 | 287 | 1164 | R136Q |
| 7 | FBXL13 | SLWNAIDFF | SEQ ID. 43 | SLWNAIDFS | SEQ ID. 44 | 414 | 348 | S201F |
| 4 | FHDC1 | ELQDEVYTL | SEQ ID. 45 | ELQDEAYTL | SEQ ID. 46 | 111 | 518 | A426V |
| 5 | GPX8 | LLSIVPCTV | SEQ ID. 47 | LLSIVLCTV | SEQ ID. 48 | 52 | 33 | L27P |
| 6 | KDM1B | IIGAGPAEL | SEQ ID. 49 | IIGAGPAGL | SEQ ID. 50 | 469 | 928 | G394E |
| 13 | LCP1 | NLFNRYLAL | SEQ ID. 51 | NLFNRYPAL | SEQ ID. 52 | 57 | 30 | P375L |
| 2 | LRP1B | WLTRNFYFV | SEQ ID. 53 | WLTRNLYFV | SEQ ID. 54 | 9 | 7 | L297F |
| 18 | NPC1 | MLSSVACSL | SEQ ID. 55 | VLSSVACSL | SEQ ID. 56 | 21 | 55 | V664M |
| 12 | OASL | ILNPADPTL | SEQ ID. 57 | ILDPADPTL | SEQ ID. 58 | 71 | 40 | D305N |
| 5 | PCDHB3 | FLFLVLLFV | SEQ ID. 59 | FLFSVLLFV | SEQ ID. 60 | 6 | 3 | S704L |
| 5 | PCDHB11 | MLLEISENS | SEQ ID. 61 | MLLEIPENS | SEQ ID. 62 | 252 | 210 | P143S |
| X | PHKA2 | LLSIIFFPA | SEQ ID. 63 | LLSIISFPA | SEQ ID. 64 | 23 | 25 | S264F |
| 6 | PTPRK | PLANSIWNV | SEQ ID. 65 | PLANPIWNV | SEQ ID. 66 | 34 | 106 | P137S |
| 5 | SH3RF2 | HIVEISTPV | SEQ ID. 67 | HMVEISTPV | SEQ ID. 68 | 27 | 6 | M320I |
| 3 | TKT | AMFWSVPTV | SEQ ID. 69 | AMFRSVPTS | SEQ ID. 70 | 4 | 1525 | R438W |
| 1 | TMEM48 | CLNEYHLFL | SEQ ID. 71 | CLNEYHLFF | SEQ ID. 72 | 23 | 3442 | F169L |
| 7 | BRAF^{d} | | | | | | | V600E |

| | | Lymph Node (Jan. 30, 2011) | | | | | | |
|---|---|---|---|---|---|---|---|---|
| | Hugo | EXOME | | | cDNA-capture | | | |
| CHR | Symbol | Alt re | Ref re | VAF^{b} | Alt re | Ref re | VAF | FPKM^{c} |
| 1 | AGMAT | 16 | 49 | 24.62 | 1 | 22 | 4.35 | 0.38 |
| 8 | ARFGEF1 | 21 | 129 | 14.00 | 64 | 240 | 20.98 | 31.37 |
| 9 | CDKN2A | 13 | 49 | 20.97 | 162 | 38 | 81.00 | 0.18 |
| 19 | CYP2S1 | 3 | 68 | 4.23 | 0 | 12 | 0.00 | 0.12 |
| 7 | FBXL13 | 12 | 44 | 21.43 | 2 | 6 | 25.00 | 0.00 |
| 4 | FHDC1 | 22 | 93 | 18.97 | 0 | 3 | 0.00 | 0.39 |
| 5 | GPX8 | 7 | 63 | 10.00 | 20 | 62 | 24.39 | 15.02 |
| 6 | KDM1B | 15 | 55 | 21.43 | 23 | 24 | 48.94 | 7.33 |
| 13 | LCP1 | 12 | 82 | 12.77 | 36 | 766 | 4.47 | 49.11 |
| 2 | LRP1B | 11 | 38 | 22.45 | 0 | 5 | 0.00 | 0.00 |
| 18 | NPC1 | 4 | 24 | 14.29 | 54 | 36 | 60.00 | 36.55 |
| 12 | OASL | 3 | 35 | 7.89 | 0 | 23 | 0.00 | 1.62 |
| 5 | PCDHB3 | 46 | 225 | 16.97 | 24 | 2 | 92.31 | 7.05 |
| 5 | PCDHB11 | 0 | 40 | 15.69 | 1 | 7 | 12.50 | 5.25 |
| X | PHKA2 | 13 | 25 | 34.21 | 13 | 21 | 38.24 | 4.60 |
| 6 | PTPRK | 14 | 89 | 13.59 | 118 | 297 | 28.43 | 0.00 |
| 5 | SH3RF2 | 14 | 61 | 18.67 | 49 | 207 | 18.99 | 10.19 |
| 3 | TKT | 10 | 45 | 18.18 | 124 | 190 | 39.49 | 0.64 |
| 1 | TMEM48 | 7 | 40 | 14.89 | 292 | 382 | 43.13 | 0.17 |
| 7 | BRAF^{d} | 10 | 55 | 15.38 | | | | |
| | | Skin (May 10, 2012) | | | | | | |
| | Hugo | EXOME | | | cDNA-capture | | | |
| CHR | Symbol | Alt re | Ref re | VAF | Alt re | Ref re | VAF | FPKM |
| 1 | AGMAT | 51 | 50 | 50.50 | 5 | 2 | 71.43 | 0.14 |
| 8 | ARFGEF1 | 109 | 154 | 41.44 | 140 | 177 | 44.03 | 34.67 |
| 9 | CDKN2A | 30 | 17 | 63.83 | 168 | 26 | 86.60 | 0.05 |
| 19 | CYP2S1 | 41 | 50 | 45.05 | 0 | 1 | 0.00 | 0.05 |
| 7 | FBXL13 | 15 | 50 | 22.39 | 0 | 1 | 0.00 | 1.61 |
| 4 | FHDC1 | 53 | 52 | 50.48 | 0 | 0 | 0.00 | 0.40 |
| 5 | GPX8 | 35 | 27 | 56.45 | 30 | 12 | 71.43 | 6.92 |
| 6 | KDM1B | 35 | 51 | 40.70 | 34 | 28 | 54.84 | 12.67 |
| 13 | LCP1 | 30 | 88 | 25.42 | 2 | 189 | 1.05 | 16.73 |
| 2 | LRP1B | 39 | 50 | 43.82 | 34 | 122 | 21.79 | 9.23 |
| 18 | NPC1 | 0 | 51 | 0.00 | 0 | 255 | 0.00 | 0.103 |
| 12 | OASL | 26 | 19 | 57.78 | 6 | 16 | 27.27 | 2.96 |
| 5 | PCDHB3 | 155 | 124 | 55.36 | 50 | 1 | 98.04 | 10.89 |
| 5 | PCDHB11 | 17 | 40 | 29.82 | 4 | 16 | 20.00 | 5.64 |
| X | PHKA2 | 31 | 5 | 86.11 | 47 | 11 | 81.03 | 6.98 |
| 6 | PTPRK | 61 | 75 | 44.85 | 172 | 144 | 54.43 | 0.02 |
| 5 | SH3RF2 | 43 | 35 | 55.13 | 101 | 71 | 58.72 | 6.82 |
| 3 | TKT | 36 | 25 | 59.02 | 129 | 122 | 51.19 | 128.54 |
| 1 | TMEM48 | 20 | 24 | 45.45 | 430 | 263 | 61.52 | 0.24 |
| 7 | BRAF^{d} | 49 | 48 | 50.52 | | | | |
| | | Skin (Jun. 6, 2013) | | | | | | |
| | Hugo | EXOME | | | cDNA-capture | | | |
| CHR | Symbol | Alt re | Ref re | VAF | Altre | Ref re | VAF | FPKM |
| 1 | AGMAT | 42 | 62 | 40.38 | 1 | 7 | 12.50 | 0.3 |
| 8 | ARFGEF1 | 31 | 103 | 23.13 | 69 | 195 | 25.84 | 34.23 |
| 9 | CDKN2A | 19 | 18 | 51.35 | 30 | 27 | 52.63 | 0.83 |
| 19 | CYP2S1 | 31 | 54 | 36.47 | 0 | 14 | 0.00 | 0.11 |
| 7 | FBXL13 | 6 | 33 | 15.38 | 0 | 6 | 0.00 | 0.00 |
| 4 | FHDC1 | 33 | 52 | 38.82 | 3 | 14 | 17.65 | 7.24 |
| 5 | GPX8 | 18 | 45 | 28.57 | 17 | 47 | 26.56 | 0.16 |
| 6 | KDM1B | 17 | 37 | 31.48 | 10 | 37 | 21.28 | 12.01 |
| 13 | LCP1 | 8 | 75 | 9.64 | 8 | 284 | 2.73 | 23.56 |
| 2 | LRP1B | 16 | 49 | 24.62 | 22 | 47 | 31.88 | 4.57 |
| 18 | NPC1 | 0 | 53 | 0.00 | 0 | 203 | 0.00 | 44.81 |
| 12 | OASL | 12 | 27 | 30.77 | 0 | 16 | 0.00 | 0.89 |
| 5 | PCDHB3 | 59 | 94 | 38.06 | 39 | 7 | 84.78 | 5.65 |
| 5 | PCDHB11 | 4 | 27 | 12.90 | 2 | 10 | 16.67 | 4.10 |
| X | PHKA2 | 11 | 12 | 45.83 | 41 | 26 | 61.19 | 7.46 |
| 6 | PTPRK | 26 | 69 | 27.37 | 58 | 149 | 38.02 | 0.23 |
| 5 | SH3RF2 | 28 | 49 | 36.36 | 47 | 76 | 38.21 | 7.63 |
| 3 | TKT | 21 | 21 | 50.00 | 173 | 338 | 33.86 | 0.93 |
| 1 | TMEM48 | 12 | 15 | 44.44 | 40 | 72 | 34.19 | 0.43 |
| 7 | BRAFd | 23 | 49 | 31.94 | | | | |

^{a}Predicted affinity as determined using NetMHC3.4 algorithm.
^{b}VAF = Variant Allelic Fraction as determined from exome sequencing. BRAF VAF are reported as these were used as comparator to assess clonality of other mutations. Candidates formulated in vaccine are shown bolded.
^{c}FPKM = Fragment Per Kilobase of transcript per Million per transcriptome as determined from cDNA-capture data
^{d}BRAF VAF values are reported and were used as comparator to interpret frequencies of remaining MM-genes.

TABLE 3
Patient Mel38

| | | | | | | Predicted Affinity (nM)^{a} | | |
|---|---|---|---|---|---|---|---|---|
| | | | | | | | | Amino |
| | Hugo | AAS- | | wild-type | | | wild- | Acid |
| CHR | Symbol | peptide | SEQ ID | peptide | SEQ ID | mutated | type | Substitution |
| 15 | AKAP13 | KLMNIQQKL | SEQ ID NO: 1 | KLMNIQQQL | SEQ ID NO: 16 | 19 | 17 | Q285K |
| 8 | ARPGEF1 | FVSALCMFL | SEQ ID NO: 2 | FVSALRMFL | SEQ ID NO: 17 | 19 | 88 | R792C |
| 17 | CCDC57 | QLCHDASTV | SEQ ID NO: 3 | QLRSDASTV | SEQ ID NO: 18 | 352 | 2265 | R353C |
| 8 | CPNE3 | LMSIIIVGV | SEQ ID NO: 4 | PMSIIIVGV | SEQ ID NO: 19 | 18 | 817 | F448L |
| 14 | DICER1 | LIMTCCVAL | SEQ ID NO: 5 | LIMTCYVAL | SEQ ID NO: 20 | 45 | 43 | Y153C |
| 16 | GLYR1 | ALVSGNQQL | SEQ ID NO: 6 | APVSGNQQL | SEQ ID NO: 21 | 273 | 25384 | P386L |
| 1 | HSD17B7 | YISKCWDYA | SEQ ID NO: 7 | YISKCWDHA | SEQ ID NO: 22 | 233 | 971 | N108Y |
| 22 | IL17RA | FIMGISILL | SEQ ID NO: 8 | FITGISILL | SEQ ID NO: 23 | 4 | 24 | T326M |
| 1 | KIP14 | IQLSWVLIA | SEQ ID NO: 9 | IQLSGVLIA | SEQ ID NO: 24 | 144 | 658 | G842W |
| 12 | MED13L | ILMTWNLRS | SEQ ID NO: 10 | ILMTGNLHS | SEQ ID NO: 25 | 259 | 1243 | G2045W |
| 3 | OR5K2 | YIFLENLAL | SEQ ID NO: 11 | YIFLGNLAL | SEQ ID NO: 26 | 55 | 38 | G64E |
| 11 | OR8B3 | QLSCISTYV | SEQ ID NO: 12 | QLSCTSTYV | SEQ ID NO: 27 | 18 | 35 | T190I |
| 11 | PSKCDBP | CLPPQTLAA | SEQ ID NO: 73 | CLSPQTLAA | SEQ ID NO: 74 | 81 | 694 | S153F |
| 5 | SEC24A | FLYNLLTRV | SEQ ID NO: 13 | FLYNPLTRV | SEQ ID NO: 28 | 4 | 6 | P469L |
| 17 | TAOK1 | MMAPEVILV | SEQ ID NO: 14 | MMAPEVILA | SEQ ID NO: 29 | 7 | 40 | A196V |
| 6 | UTRN | QLDKCSAFV | SEQ ID NO: 15 | QLDQCSAFV | SEQ ID NO: 30 | 21 | 22 | Q1058K |
| 2 | WDR35 | FLNCNSSRL | SEQ ID NO: 75 | SLNCNSSRL | SEQ ID NO: 76 | 38 | 616 | S550F |
| 7 | ZYX | SLKGTSFIV | SEQ ID NO: 77 | PLEGTSFIV | SEQ ID NO: 78 | 64 | 5774 | P329S |
| 7 | BRAF^{d} | | | | | | | V600E |

Acilla (Apr. 19, 2012)

| | | EXOME | | | | | | |
|---|---|---|---|---|---|---|---|---|
| | Hugo | Alt_ | Ref_ | | cDNA-capture | | | |
| CHR | Symbol | reads | reads | VAF^{b} | Alt_reads | Ref re | VAF | FPKM^{c} |
| 15 | AKAP13 | 20 | 50 | 28.57 | 4 | 13 | 23.53 | 54.3 |
| 8 | ARPGEF1 | 23 | 81 | 22.12 | 60 | 161 | 27.15 | 7.1 |
| 17 | CCDC57 | 35 | 41 | 26.78 | 53 | 351 | 13.12 | 9.5 |
| 8 | CPNE3 | 31 | 127 | 19.62 | 113 | 536 | 17.41 | 14.3 |
| 14 | DICER1 | 10 | 21 | 32.26 | 2 | 4 | 33.33 | 4.1 |
| 16 | GLYR1 | 21 | 25 | 45.65 | 124 | 150 | 45.26 | 155.5 |
| 1 | HSD17B7 | 52 | 183 | 22.13 | 68 | 228 | 22.97 | 29.7 |
| 22 | IL17RA | 12 | 28 | 30 | 4 | 26 | 13.33 | 1.9 |
| 1 | KIP14 | 23 | 68 | 25.27 | 5 | 25 | 16.67 | 2.2 |
| 12 | MED13L | 12 | 8 | 60 | 71 | 81 | 46.71 | 8.8 |
| 3 | OR5K2 | 57 | 64 | 47.11 | 3 | 0 | 100 | 0.1 |
| 11 | OR8B3 | 15 | 0 | 100 | 13 | 1 | 92.88 | 0.6 |
| 11 | PSKCDBP | 13 | 0 | 100 | 24 | 0 | 100.00 | 0.0 |
| 5 | SEC24A | 22 | 25 | 46.81 | 50 | 56 | 46.73 | 2.6 |
| 17 | TAOK1 | 23 | 33 | 41.07 | 23 | 29 | 44.23 | 3.0 |
| 6 | UTRN | 22 | 0 | 100 | 44 | 1 | 97.78 | 6.9 |
| 2 | WDR35 | 34 | 15 | 69.39 | 90 | 41 | 58.7 | 15.2 |
| 7 | ZYX | 18 | 48 | 27.27 | 26 | 67 | 27.96 | 6.7 |
| 7 | BRAF^{d} | 58 | 14 | 80 | | | | |
| | | Breast (Feb. 14, 2013) | | | | | | |
| | | EXOME | | | cDNA-capture | | | |
| | Hugo | Alt_ | Ref_ | | Alt_ | Ref_ | | |
| CHR | Symbol | reads | reads | VAF | reads | reads | VAF | FPKM |
| 15 | AKAP13 | 19 | 117 | 14.0 | 31 | 101 | 23.5 | 1.47 |
| 8 | ARPGEF1 | 46 | 194 | 19.2 | 206 | 1161 | 15.1 | 23.73 |
| 17 | CCDC57 | 29 | 92 | 24.0 | 91 | 210 | 30.2 | 0.79 |
| 8 | CPNE3 | 42 | 229 | 15.5 | 608 | 2833 | 17.5 | 0.29 |
| 14 | DICER1 | 24 | 37 | 39.3 | 65 | 56 | 49.6 | 7.21 |
| 16 | GLYR1 | 54 | 57 | 48.7 | 384 | 513 | 42.8 | 35.63 |
| 1 | HSD17B7 | 102 | 467 | 17.9 | 411 | 1644 | 20.0 | 0.11 |
| 22 | IL17RA | 35 | 77 | 31.3 | 33 | 90 | 26.8 | 0.22 |
| 1 | KIP14 | 35 | 132 | 22.0 | 22 | 74 | 20.9 | 3.63 |
| 12 | MED13L | 20 | 25 | 44.4 | 156 | 202 | 43.6 | 13.64 |
| 3 | OR5K2 | 125 | 227 | 35.5 | 0 | 20 | 0.0 | 0.00 |
| 11 | OR8B3 | 40 | 21 | 65.8 | 3 | 0 | 100.0 | 0.35 |
| 11 | PSKCDBP | 21 | 6 | 77.8 | 161 | 11 | 93.6 | 0.64 |
| 5 | SEC24A | 33 | 55 | 37.5 | 127 | 172 | 42.5 | 1.34 |
| 17 | TAOK1 | 37 | 83 | 30.8 | 185 | 339 | 35.3 | 11.32 |
| 6 | UTRN | 35 | 25 | 58.3 | 207 | 46 | 81.5 | 15.94 |
| 2 | WDR35 | 56 | 50 | 52.8 | 389 | 247 | 61.8 | 0.04 |
| 7 | ZYX | 27 | 104 | 20.6 | 115 | 405 | 22.1 | 14.64 |
| 7 | BRAF^{d} | 103 | 45 | 69.38 | | | | |
| | | Abd. wall(Apr. 16, 2013) | | | | | | |
| | | EXOME | | | cDNA-capture | | | |
| | Hugo | Alt_ | Ref_ | | Alt_ | Ref_ | | |
| CHR | Symbol | reads | reads | VAF | reads | reads | VAF | FPKM |
| 15 | AKAP13 | 39 | 116 | 25.16 | 13 | 37 | 26.00 | 0.14 |
| 8 | ARPGEF1 | 29 | 219 | 11.65 | 56 | 460 | 10.79 | 17.51 |
| 17 | CCDC57 | 32 | 85 | 27.35 | 45 | 170 | 20.93 | 2.23 |
| 8 | CPNE3 | 38 | 203 | 16.12 | 342 | 1684 | 16.86 | 2.27 |
| 14 | DICER1 | 18 | 40 | 31.03 | 17 | 27 | 31.48 | 8.05 |
| 16 | GLYR1 | 38 | 50 | 43.18 | 214 | 246 | 38.21 | 32.52 |
| 1 | HSD17B7 | 100 | 443 | 18.42 | 195 | 896 | 17.86 | 0.20 |
| 22 | IL17RA | 22 | 69 | 24.18 | 7 | 83 | 7.78 | 0.27 |
| 1 | KIP14 | 28 | 143 | 16.28 | 6 | 21 | 22.22 | 2.10 |
| 12 | MED13L | 14 | 32 | 30.43 | 77 | 197 | 26.10 | 14.97 |
| 3 | OR5K2 | 105 | 246 | 29.83 | 14 | 8 | 63.64 | 0.47 |
| 11 | OR8B3 | 17 | 52 | 24.64 | 1 | 2 | 33.33 | 0.25 |
| 11 | PSKCDBP | 17 | 9 | 65.38 | 112 | 13 | 88.89 | 1.94 |
| 5 | SEC24A | 19 | 60 | 24.05 | 34 | 134 | 20.12 | 0.39 |
| 17 | TAOK1 | 30 | 65 | 31.58 | 78 | 191 | 29.00 | 8.28 |
| 6 | UTRN | 23 | 36 | 38.98 | 58 | 42 | 57.43 | 12.56 |
| 2 | WDR35 | 59 | 62 | 48.76 | 239 | 365 | 59.16 | 0.02 |
| 7 | ZYX | 22 | 81 | 19.47 | 44 | 477 | 8.43 | 20.16 |
| 7 | BRAF^{d} | 69 | 56 | 55.20 | | | | |

^{a}Predicted affinity as determined using NetMHC3.4 algorithm.
^{b}VAF = variant Allelic Fraction as determined from exome sequencing. BRAF VAF are reported as tehse were used as comparator to assess clonality of other mutations.
^{c}FPKM = Fragment Per Kilobase of transcript per Million per transcriptome as determined from cDNA-capture data.
^{d}BRAF VAF values are reported and were used as comparator to interpret frequencies of remaining MM-genes

TABLE 4
MEL 218

| | | | | | | Predicted Affinity (nM) | | |
|---|---|---|---|---|---|---|---|---|
| | | | | | | | | Amino |
| | | | | wild- | | | | Acid |
| | Hugo | AAS- | | type | | | wild- | Substitution |
| CHR | Symbol | peptide | SEQ ID | peptide | SEQ ID | mutated | type | (AAS) |
| X | ABCD1 | GMHLLITGL | SEQ ID NO: 79 | GMHLLITGP | SEQ ID NO: 80 | 202 | 18419 | P508L |
| 2 | ALMS1 | VLAVSVLAA | SEQ ID NO: 81 | VSAVSVLAA | SEQ ID NO: 82 | 170 | 13703 | S934L |
| 15 | BTBD1 | FMLLTQARI | SEQ ID NO: 83 | FMLLTQARL | SEQ ID NO: 84 | 52 | 36 | L189T |
| 9 | CDC14B | IQYFRNHNV | SEQ ID NO: 85 | IQYFKNHNV | SEQ ID NO: 86 | 93 | 93 | K253R |
| 15 | DMXL2 | SVMIMAFSV | SEQ ID NO: 87 | SDMIMAFSV | SEQ ID NO: 88 | 19 | 6986 | D2662V |
| 1 | EIF2B3 | SISKPLLPV | SEQ ID NO: 89 | STPKPLLPV | SEQ ID NO: 90 | 105 | 166 | P24S |
| 1 | EXOC8 | IILVAVPHV | SEQ ID NO: 91 | IILVAVQHV | SEQ ID NO: 92 | 25 | 143 | Q656P |
| 22 | FBXO7 | LMLESGYIL | SEQ ID NO: 93 | LMLESGYIP | SEQ ID NO: 94 | 10 | 5952 | P100L |
| 7 | GET4 | AVDDGKLTV | SEQ ID NO: 95 | AVDGGKLTV | SEQ ID NO: 96 | 357 | 1067 | G196D |
| 15 | HERC1 | SLLLLSVSV | SEQ ID NO: 97 | SLLLLPVSV | SEQ ID NO: 98 | 20 | 24 | P1074S |
| 6 | HLA-DRB5 | YMAELTVTL | SEQ ID NO: 99 | YMAKLTVTL | SEQ ID NO: 100 | 4 | 7 | K14E |
| 8 | KAT6A | KLSREIKPV | SEQ ID NO: 101 | KLSREIMPV | SEQ ID NO: 102 | 62 | 6 | M1180K |
| 4 | LARP7 | AVIDAYTEI | SEQ ID NO: 103 | AVINAYTEI | SEQ ID NO: 104 | 213 | 775 | N515D |
| 7 | MRPS17 | VLLRALPVL | SEQ ID NO: 105 | VLLRALPVP | SEQ ID NO: 106 | 24 | 11696 | P167L |
| 2 | MRPS5 | HLYASLSRA | SEQ ID NO: 107 | HPYASLSRA | SEQ ID NO: 108 | 116 | 23536 | P59L |
| 12 | OSBPL8 | FCFKLSHPL | SEQ ID NO: 109 | FCFKLFHPL | SEQ ID NO: 110 | 174 | 126 | P213S |
| 8 | PABPC1 | MLGEQLFPL | SEQ ID NO: 111 | MLGERLFPL | SEQ ID NO: 112 | 3 | 3 | R520Q |
| 3 | PLA1A | FIWGDAPPT | SEQ ID NO: 113 | SIWGDAPPT | SEQ ID NO: 114 | 41 | 744 | S6F |
| 17 | RNASEK | RLLCPPARA | SEQ ID NO: 115 | RPLCPPARA | SEQ ID NO: 116 | 432 | 22016 | P10L |
| 20 | SMOX | KLANPLPYT | SEQ ID NO: 117 | KLAKPLPYT | SEQ ID NO: 118 | 38 | 63 | K499N |
| 1 | SRP9 | IMAHCILDL | SEQ ID NO: 119 | IIAHCILDL | SEQ ID NO: 120 | 22 | 250 | I64M |
| 13 | TPP2 | SLAETFLET | SEQ ID NO: 121 | SLAETFWET | SEQ ID NO: 122 | 82 | 17 | W1168L |
| 1 | VANGL1 | FVFCALLLV | SEQ ID NO: 123 | FVFRALLLV | SEQ ID NO: 124 | 6 | 10 | R186C |
| 16 | ZFP90 | FTQEKWYHV | SEQ ID NO: 125 | FTQEEWYHV | SEQ ID NO: 126 | 22 | 20 | E27K |
| 7 | BRAF^{d} | | | | | | | V600E |

| | | Lymph Node (Apr. 4, 2005) | | | | | | |
|---|---|---|---|---|---|---|---|---|
| | Hugo | EXOME | | | cDNA-capture | | | |
| CHR | Symbol | Alt re | Ref re | VAF^{b} | Alt_reads | Ref reads | VAF | FPKM^{c} |
| X | ABCD1 | 23 | 38 | 37.7 | 156 | 12 | 92.86 | 10.65 |
| 2 | ALMS1 | 5 | 11 | 31.25 | 20 | 20 | 50 | 5.74 |
| 15 | BTBD1 | 6 | 17 | 26.09 | 170 | 358 | 32.14 | 18.84 |
| 9 | CDC14B | 6 | 67 | 8.11 | 27 | 136 | 16.56 | 10.73 |
| 15 | DMXL2 | 10 | 46 | 17.86 | 102 | 704 | 12.64 | 50.71 |
| 1 | EIF2B3 | 5 | 24 | 17.24 | 55 | 111 | 32.93 | 13.83 |
| 1 | EXOC8 | 7 | 26 | 21.21 | 145 | 300 | 32.37 | 4.83 |
| 22 | FBXO7 | 12 | 45 | 21.05 | 900 | 1597 | 36.04 | 87.45 |
| 7 | GET4 | 20 | 27 | 42.55 | 57 | 51 | 52.78 | 5.2 |
| 15 | HERC1 | 12 | 55 | 17.91 | 68 | 162 | 29.57 | 71.99 |
| 6 | HLA-DRB5 | 81 | 85 | 48.8 | 573 | 1645 | 25.8 | 247.95 |
| 8 | KAT6A | 25 | 116 | 17.73 | 261 | 463 | 36 | 27.21 |
| 4 | LARP7 | 6 | 36 | 14.29 | 30 | 50 | 37.5 | 10.15 |
| 7 | MRPS17 | 5 | 71 | 6.58 | 29 | 75 | 27.88 | 1.48 |
| 2 | MRPS5 | 10 | 58 | 14.49 | 60 | 125 | 32.43 | 14.55 |
| 12 | OSBPL8 | 6 | 35 | 14.63 | 341 | 614 | 35.63 | 105.47 |
| 8 | PABPC1 | 16 | 44 | 26.67 | 4073 | 11235 | 26.6 | 1180.59 |
| 3 | PLA1A | 18 | 79 | 18.56 | 4 | 10 | 28.57 | 4.07 |
| 17 | RNASEK | 9 | 58 | 13.43 | 9 | 18 | 33.33 | 109.67 |
| 20 | SMOX | 131 | 0 | 100 | 11 | 50 | 18.03 | 3.01 |
| 1 | SRP9 | 0 | 58 | 0* | 43 | 41 | 51.19 | 2.31 |
| 13 | TPP2 | 10 | 98 | 9.26 | 98 | 265 | 26.92 | 25.93 |
| 1 | VANGL1 | 22 | 159 | 12.15 | 289 | 714 | 28.76 | 26.52 |
| 16 | ZFP90 | 11 | 70 | 13.58 | 22 | 53 | 29.33 | 4.29 |
| 7 | BRAF^{d} | 13 | 47 | 21.67 | | | | |

^{a}Predicted affinity as determined using NetMHC3.4 algorithm.
^{d}BRAF VAF values are reported and were used as comparator to interpret frequencies of remaining MM-genes.
(*) Expression of mutated gene was validated by cDNA-capture and Sanger sequencing.
Candidates formulated in vaccine are shown in bold.

TABLE 5
Analysis of HLA-A*02: 01 restricted AAS-directed CD8+ T cell responses

| | | | | | | | | | Recog- | |
|---|---|---|---|---|---|---|---|---|---|---|
| | | Amino | | | | Experi- | | | nition | |
| | | Acid | | | | mental | | | of | |
| | | Substi- | | | Predicted | Affinity | Sponta- | | pro- | |
| | Hugo | tution | Mutated | | affinity | log(IC50, | neous | Immuno- | cessed | Antigenic |
| Patient | symbol | (AAS) | Peptide^{a} | SEQ ID | (nM) | nM)^{b} | Immunity^{c} | genicity | antigen | Determinant^{d} |
| MEL21 | ARFGEF1 | F1637L | QTIDNIVFL | SEQ ID 37 | 67 | 3.19 | No | No | | |
| | CDKN2A | K153K | KMIGNHLWV | SEQ ID 39 | 14 | 3.18 | No | Yes | Yes | SUBDOMINANT |
| | GPY8 | L27P | LLSIVPCTV | SEQ ID 47 | 52 | 3.09 | No | No | | |
| | KDM1B | G394E | IIGAGPAEV* | SEQ ID 166 | 111 | 3.82 | No | No | | |
| | PHKA2 | S264F | LLSIIFFPA | SEQ ID 63 | 23 | 3.90 | No | No | | |
| | TKT | R438W | AMFWSVPTV* | SEQ ID 69 | 4 | 2.35 | No | Yes | Yes | SUBDOMINANT |
| | TMEM48 | P169L | CLNEYHLFL | SEQ ID 71 | 23 | 3.09 | Yes | Yes | Yes | DOMINANT |
| MKL38 | AKAP13 | Q285K | KLMNIQQKL | SEQ ID 1 | 19 | 3.07 | No | Yes | Yes | SUBDOMINANT |
| | ARFGEF1 | R782C | FVSALQMFL | SEQ ID 2 | 19 | 3.18 | No | No | | |
| | HSD1787 | H108Y | YISKCWDYA | SEQ ID 7 | 233 | 4.28 | No | No | | |
| | OR8B3 | T190I | QLSCISTYV | SEQ ID 12 | 18 | 3.10 | No | Yes | No | CRYPTIC |
| | PRKCDEP | S153F | CLFPQTLAA | SEQ ID 73 | 81 | 3.53 | No | No | | |
| | SKC24A | P469L | FLYNLLTRV | SEQ ID 13 | 4 | 2.68 | Yes | Yes | Yes | DOMINANT |
| | UTRN | Q1058K | QLDKCSAFV | SEQ ID 15 | 21 | 3.36 | No | No | | |
| MEL218 | EXOC8 | Q656P | IILVAVPKV | SEQ ID 91 | 25 | 3.06 | Yes | Yes | Yes | DOMINANT |
| | LARP7 | N518D | AVIDAYTEI | SEQ ID 103 | 213 | 4.41 | No | No | | |
| | MRPS5 | P59L | HLYASLSRV* | SEQ ID 167 | 19 | 3.28 | No | Yes | No | CRYPTIC |
| | MRPS17 | P167L | VLLRALPVL | SEQ ID 105 | 24 | 3.05 | No | No | | |
| | PABPC1 | R520Q | MLGEQLFPL | SEQ ID 111 | 3 | 2.35 | No | Yes | Yes | SUBDOMINANT |
| | SMOX | K499N | KLANPLPYT | SEQ ID 117 | 134 | 3.73 | No | No | | |
| | SRP8 | I64M | IMAHCILDL | SEQ ID 119 | 37 | 4.02 | No | No | | |

^{a}Mutated residues are underlined and peptides that elicited immune responses are itacized (naturally-occurring) and bold (vaccine-induced).
*Indicates anchor-modified peptides at P9 (Tables 2-4).
^{b}Affinity experimentally determined using fluorescence polarization-based competitive peptide-binding assay, high affinity binding peptides in this assay are log(IC50; nM) <3.7 (11).
^{c}As determined by immune monitoring assay (FIG. 31, FIG. 30B),
^{d}Antigenic determinant classification according to Sercarz et al. Annu. Rev. Immunol. 11, 729-766 (1993).

TABLE 6
Composition of TMC constructs

| | | Mut AA | | |
|---|---|---|---|---|
| Tumor | Gene | Position | Nucleotide sequence* | Seq ID No. |
| MEL21 | ARFGEF1 | 1637 | CAGCTGGAGCTGATCCAGACGATAGACAACATCGTGTTCGTGCCTGCAACTAGTAAG | SEQ ID NO: 168 |
| | GPX8 | 27 | AAAGTTTTCGCTGTCTTGCTCTCCATTGTGCCGTGCACAGTGACACTTTTTCTGCTT | SEQ ID NO: 169 |
| | KDM1B | 384 | AACAAGAGCGTGATAATTATAGGAGCTGGCCCAGCAGAAGTGGCAGCAGCTAGACAA | SEQ ID NO: 170 |
| | PHKA2 | 264 | GAGATCGATGCTGGACTGCTTAGCATAATCTTTTTTCCTGCTTTTGCGGTAGAGGAT | SEQ ID NO: 171 |
| | TKT | 438 | GCGCTGGAAGACCTGGCTATGTTTTGGTCAGTGCCCACAGTGACAGTCTTCTACCCTTCTGAT | SEQ ID NO: 172 |
| | TMEM48 | 168 | CCTGCAGCTCAGACCTGTCTCAACGAGTATCACCTGTTCCTGCTTCTCACAGGTGCC | SEQ ID NO: 173 |
| | CDKN2A | 153 | GCTGAGGGACCCTCCAAAATGATAGGTAACCATCTGTGGGTATGTCGGAGTCGCCAT | SEQ ID NO: 174 |
| MEL38 | AKAP13 | 285 | ACTGGCCCTATTTTTAAGCTCATGAATATCCAGCAAAAGCTTATGAAAACAAATCTGAAG | SEQ ID NO: 175 |
| | ARFGEF1 | 782 | TTTAGCGGAAAAGATTTTGTGAGCGCACTCTGCATGTTTCTCGAGGGATTCAGGCTGCCA | SEQ ID NO: 176 |
| | PRKCDEP | 153 | GTGCCCAGTCATGCGTGTCTTTTCCCCCAAACTCTGGCCGCTGAGGAGGAGGGCGAGGTG | SEQ ID NO: 177 |
| | SKC24A | 469 | GATGTGCCAGAGGAGTTTCTCTATAATCTGCTTACACGCGTCTACGGAGAGGCACACCGG | SEQ ID NO: 178 |
| | UTRN | 1058 | GCTGGATTGCAGCGGCAGCTGGACAAATGCAGCGCATTCGTAAATGAGATCGAAACCATA | SEQ ID NO: 179 |
| | OR8B3 | 190 | ATCCTGCCACTGCTGCAACTGTCTTGCATTCTACCTACGTGAATGAAGTCGTGGTGCTC | SEQ ID NO: 180 |
| | HSD1787 | 108 | TCCGAGATCAGACCATACATTAGCAAGTGCTGGGACTATGCC | SEQ ID NO: 181 |
| MEL218 | MRPS17 | 167 | ACAGTGGGGGACATTGTGCTGCTCCGAGCACTGCCCGTACTTCGAGCAAAACACGTGAAG | SEQ ID NO: 182 |
| | MRPS5 | 59 | GGCACACGGGACACACATCTGTATGCCAGCTTGAGCCGCGCACTCCAAACACAGTGCTGC | SEQ ID NO: 183 |
| | SRP8 | 64 | CAATGCTCCGGTATGATCATGGCCCACTGTATCCTCGACTTGTTGGCAGCAGCGGGCCC | SEQ ID NO: 184 |
| | LARP7 | 528 | CCAGAGGACGCACAGGCAGTGATCGACGCCTACACCGAGATAAACAAGAAACATTGCTGG | SEQ ID NO: 185 |
| | EXOC8 | 654 | GAATCCCTGGTCGAGATCATCCTGGTAGCTGTTCCACATGTCGATTACAGCCTTAGGTGT | SEQ ID NO: 186 |
| | SMOX | 499 | GGTGCCGATGTCGAAAAGCTCGCCAACCCTCTCCCTTATACGGAATCAAGCAAAACCGCG | SEQ ID NO: 187 |
| | PABPC1 | 538 | CCACAGGAGCAAAAAATGTTGGGCGAACAATTGTTCCCGCTGATTCAGGCGATGCACCCG | SEQ ID NO: 188 |
| Contol | | Mut AA | | |
| Ag | Gene | Position | Nucleotide sequence | Seq ID No. |
| G380 | GP100 | N/A | GTGGTGACACACACCTATCTCGAGCCGGGCCCCGTGACAGCCCAGGTAGTTCTGCAGGCC | SEQ ID NO: 189 |
| SVG8 | KNV | N/A | GCTTGGGATTTCGGGAGCGTGGGTGGCGTCTTCACATCTGTTGGCAAGGCAGTGCATCAG | SEQ ID NO: 190 |

*nucleotide sequences encoding 19-21-mer amino acid sequence containing missense mutation targeted by peptides included in vaccine.

TABLE 7
Reference TCR CDR3 library from dominant TMEM4 F169L expanded CD8+ T cells (MEI

| CDR3 amino acid sequence | SEQ ID | TCRBV | TCRBD | TCRBJ | Frequency | Read Counts |
|---|---|---|---|---|---|---|
| CASSQDLSGGVYYGYTF | EQ ID NO: 19 | TCRBV04-01 | | TCRBJ01-02 | 18.24 | 494191 |
| CSTLLAGGGDEQYV | EQ ID NO: 19 | TCRBV29-01 | TCRBD02 | TCRBJ02-07 | 3.96 | 107162 |
| CASSPTGLGETQYF | EQ ID NO: 19 | TCRBV10-02 | TCRBD01 | TCRBJ02-05 | 2.97 | 80581 |
| CSAPPGPLAHTQYF | EQ ID NO: 19 | TCRBV20 | TCRBD02 | TCRBJ02-03 | 2.14 | 58087 |
| CASSFKGTGPNQPQHF | EQ ID NO: 19 | TCRBV27-01 | TCRBD01 | TCRBJ01-05 | 0.98 | 26493 |
| CASSFGGPPNTGELFF | EQ ID NO: 19 | TCRBV06 | TCRBD02 | TCRBJ02-02 | 0.88 | 23788 |
| CASSIGPVNTEAFF | EQ ID NO: 19 | TCRBV19-01 | TCRBD01 | TCRBJ01-01 | 0.21 | 5787 |
| CASSVAASPSGNTIYF | EQ ID NO: 19 | TCRBV09-01 | | TCRBJ01-03 | 0.19 | 5051 |
| CASSPYRAGYEQYF | EQ ID NO: 19 | TCRBV03 | TCRBD01 | TCRBJ02-07 | 0.11 | 3056 |
| CASSRTGITDTQYF | EQ ID NO: 20 | TCRBV03 | TCRBD01 | TCRBJ02-03 | 0.06 | 1619 |

TABLE 8
Reference TCRB CDR3 library from subdominant TXT R438W expanded CD8+ T cells (MEL21)

| CDR3 amino acid sequence | Seq ID No. | TCRBV | TCRBD | TCRBJ | Frequency | Read Counts |
|---|---|---|---|---|---|---|
| CASSIASGIYEQYF | SEQ ID NO: 201 | TCRBV19-01 | TCRBD02 | TCRBJ02-07 | 4.97 | 112412 |
| CASSISSSEKLFF | SEQ ID NO: 202 | TCRBV19-01 | TCRBD02 | TCRBJ01-04 | 4.79 | 108219 |
| CASSLVVGLALEQYF | SEQ ID NO: 203 | TCRBV12 | TCRBD02 | TCRBJ02-07 | 2.96 | 66048 |
| CASSFWGLSTEAFF | SEQ ID NO: 204 | TCRBV12 | TCRBD02 | TCRBJ01-01 | 2.75 | 62085 |
| CASSSDLYEQYF | SEQ ID NO: 205 | TCRBV05-04 | | TCRBJ02-03 | 2.38 | 53716 |
| CASSQEVGSGNTIYF | SEQ ID NO: 206 | TCRBV04-03 | | TCRBJ01-03 | 2.00 | 45241 |
| CASSSAGGGGNTIYF | SEQ ID NO: 207 | TCRBV07-08 | TCRBD01 | TCRBJ01-07 | 1.97 | 44623 |
| CASSIAGGYEQYV | SEQ ID NO: 208 | TCRBV19-01 | TCRBD01 | TCRBJ02-01 | 1.86 | 42081 |
| CSVVGGLLEAFF | SEQ ID NO: 209 | TCRBV19-01 | TCRBD02 | TCRBJ01-01 | 1.84 | 41524 |
| CASSSDWGLMNTEAFF | SEQ ID NO: 210 | TCRBV05-06 | TCRBD01 | TCRBJ01-01 | 1.78 | 40297 |
| CASSAVDRVTSYNEQFF | SEQ ID NO: 211 | TCRBV27-01 | TCRBD01 | TCRBJ02-03 | 1.67 | 37852 |
| CASSLIAGNSDTQYF | SEQ ID NO: 212 | TCRBV27-01 | TCRBD02 | TCRBJ02-05 | 1.64 | 37136 |
| CASRLTAGEYQETQYF | SEQ ID NO: 213 | TCRBV12-02 | TCRBD02 | TCRBJ02-02 | 1.64 | 36999 |
| CASSLWDYGYTF | SEQ ID NO: 214 | TCRBV05-06 | | TCRBJ01-01 | 1.59 | 35919 |
| CASSLWGVGTEAFF | SEQ ID NO: 215 | TCRBV12 | TCRBD02 | TCRBJ01-06 | 1.54 | 34759 |
| CASSYFGVNSPLHF | SEQ ID NO: 216 | TCRBV06 | TCRBD02 | TCRBJ01-01 | 1.48 | 33424 |
| CATSALAGQGRDEQFF | SEQ ID NO: 217 | TCRBV24 | TCRBD01 | TCRBJ02-03 | 1.42 | 32032 |
| CASSRLAGTDTQYF | SEQ ID NO: 218 | TCRBV12 | TCRBD02 | TCRBJ02-01 | 1.36 | 30644 |
| CASSFPGYGLNTEAFF | SEQ ID NO: 219 | TCRBV06 | TCRBD02 | TCRBJ01-01 | 1.59 | 36045 |
| CASSVLAGGLDTQYF | SEQ ID NO: 220 | TCRBV10-02 | TCRBD02 | TCRBJ02-03 | 1.15 | 26035 |
| CASSYMLQTFNTEAFF | SEQ ID NO: 221 | TCRBV06 | | TCRBJ01-01 | 1.00 | 22716 |
| CASSPGLLAGGSSWETQYF | SEQ ID NO: 222 | TCRBV07-02 | TCRBD02 | TCRBJ02-05 | 0.99 | 22276 |
| CASTSTPGQVGQPQHF | SEQ ID NO: 223 | TCRBV27-01 | TCRBD01 | TCRBJ01-05 | 0.95 | 21583 |
| CASKGLAGAYTDTQYF | SEQ ID NO: 224 | TCRBV12 | TCRBD02 | TCRBJ02-03 | 0.87 | 19584 |
| CASSLGGNEQYF | SEQ ID NO: 225 | TCRBV07-08 | | TCRBJ02-07 | 0.86 | 19499 |
| CASSFTAGLNTEAFF | SEQ ID NO: 226 | TCRBV12 | TCRBD01 | TCRBJ01-01 | 0.83 | 18659 |
| CASSLVWGLGTEAFF | SEQ ID NO: 227 | TCRBV28-01 | | TCRBJ01-01 | 0.80 | 18100 |
| CASSLGLSGESF | SEQ ID NO: 228 | TCRBV07-08 | TCRBD02 | unresolved | 0.78 | 17740 |
| CASSKLAGGLDTQYF | SEQ ID NO: 229 | TCRBV10-02 | TCRBD02 | TCRBJ02-03 | 0.78 | 17662 |
| CASTHRTGLNTEAFF | SEQ ID NO: 230 | TCRBV12 | TCRBD01 | TCRBJ01-01 | 0.77 | 17470 |
| CASSIGGQEETQYF | SEQ ID NO: 231 | TCRBV03 | TCRBD01 | TCRBJ02-05 | 0.76 | 17163 |
| CASSLEIVGETEAFF | SEQ ID NO: 232 | TCRBV05-06 | | TCRBJ01-01 | 0.68 | 15460 |
| CASSISGGYEQYV | SEQ ID NO: 233 | TCRBV19-01 | TCRBD01 | TCRBJ02-07 | 0.68 | 15403 |
| CSARTLAGFTDTQYF | SEQ ID NO: 234 | TCRBV20 | TCRBD02 | TCRBJ02-03 | 0.65 | 14669 |
| CASSDLLTGELFF | SEQ ID NO: 235 | TCRBV06-01 | TCRBD03 | TCRBJ02-02 | 0.58 | 13155 |
| CASSSGLAGYLM | SEQ ID NO: 236 | TCRBV07-08 | TCRBD02 | TCRBJ02-03 | 0.55 | 12339 |
| CASSHRTTDEETQYF | SEQ ID NO: 237 | TCRBV23-01 | TCRBD01 | TCRBJ02-05 | 0.54 | 12253 |
| CASSYPGYGLNTEAFF | SEQ ID NO: 238 | TCRBV06 | | TCRBJ01-01 | 0.49 | 11037 |
| CASSLDLYEQYF | SEQ ID NO: 239 | TCRBV05-04 | | TCRBJ02-07 | 0.44 | 9958 |
| CASSWTGFGLNTEAFF | SEQ ID NO: 240 | TCRBV06 | TCRBD01 | TCRBJ01-01 | 0.44 | 9860 |
| CASSLITGLSYEQYF | SEQ ID NO: 241 | TCRBV12 | TCRBD01 | TCRBJ02-07 | 0.42 | 9469 |
| CASSTWTGMNTEAFF | SEQ ID NO: 242 | TCRBV28-01 | TCRBD01 | TCRBJ01-01 | 0.40 | 9127 |
| CASSELWGAGDNEQFF | SEQ ID NO: 243 | TCRBV10-02 | TCRBD02 | TCRBJ02-01 | 0.39 | 8722 |
| CASSFITTSLNVEQYF | SEQ ID NO: 244 | TCRBV28-01 | TCRBD02 | TCRBJ02-07 | 0.38 | 8666 |
| CSAQQGIQPQHF | SEQ ID NO: 245 | TCRBV20 | TCRBD01 | TCRBJ01-05 | 0.38 | 8478 |
| CASSLVGGLAETQYF | SEQ ID NO: 246 | TCRBV27-01 | | TCRBJ02-05 | 0.35 | 7817 |
| CASSFSGGLTHEQYV | SEQ ID NO: 247 | TCRBV06 | TCRBD02 | TCRBJ02-07 | 0.35 | 7808 |
| CASSLGAGEQYF | SEQ ID NO: 248 | TCRBV07-08 | | TCRBJ02-07 | 0.33 | 7414 |
| CASSPIFGLTNEQYF | SEQ ID NO: 249 | TCRBV02-01 | TCRBD02 | TCRBJ02-07 | 0.31 | 6910 |
| CASSYFGGEQFF | SEQ ID NO: 250 | TCRBV06 | TCRBD02 | TCRBJ02-01 | 0.30 | 6856 |
| CASSQDWGLNYEQYF | SEQ ID NO: 251 | TCRBV04-01 | | TCRBJ02-07 | 0.30 | 6776 |
| CASSTSGGYEQYF | SEQ ID NO: 252 | TCRBV19-01 | TCRBD02 | TCRBJ02-07 | 0.28 | 6396 |
| CASSRLAGGLDTQYF | SEQ ID NO: 253 | TCRBV10-02 | TCRBD02 | TCRBJ02-03 | 0.28 | 6392 |
| CASSGLITDTQYF | SEQ ID NO: 254 | TCRBV19-01 | TCRBD02 | TCRBJ02-03 | 0.26 | 5848 |
| CSARELAGFQETQYF | SEQ ID NO: 255 | TCRBV20 | TCRBD02 | TCRBJ02-05 | 0.25 | 5732 |
| CSPIRGIEQYV | SEQ ID NO: 256 | TCRBV20-01 | TCRBD02 | TCRBJ02-07 | 0.24 | 5486 |
| CAIGPQGGFYEQYF | SEQ ID NO: 257 | TCRBV10-02 | TCRBD01 | TCRBJ02-07 | 0.24 | 5364 |
| CATSSAILAGVKETQYF | SEQ ID NO: 258 | TCRBV15-01 | TCRBD02 | TCRBJ02-05 | 0.24 | 5313 |
| CASSEGVGLAFEQFF | SEQ ID NO: 259 | TCRBV02-01 | TCRBD02 | TCRBJ02-01 | 0.23 | 5254 |
| CAIGLAGAYEQYF | SEQ ID NO: 260 | TCRBV10-03 | TCRBD02 | TCRBJ02-07 | 0.23 | 5123 |
| CASSSWTGLSLSFYGYTF | SEQ ID NO: 261 | TCRBV28-01 | TCRBD01 | TCRBJ01-02 | 0.22 | 5077 |
| CASSEPGTVEAFF | SEQ ID NO: 262 | TCRBV02-01 | TCRBD02 | TCRBJ01-01 | 0.21 | 4771 |
| CSVEEGIDEQYF | SEQ ID NO: 263 | TCRBV29-01 | | TCRBJ02-07 | 0.20 | 4627 |
| CASSLGAGEQFF | SEQ ID NO: 264 | TCRBV07-08 | TCRBD02 | TCRBJ02-01 | 0.20 | 4549 |
| CASSFQGGTGNTIYF | SEQ ID NO: 265 | TCRBV07-08 | TCRBD02 | TCRBJ01-03 | 0.20 | 4505 |
| CASSLALPYEQYF | SEQ ID NO: 266 | TCRBV12 | TCRBD02 | TCRBJ02-07 | 0.18 | 4029 |
| CASSPTQGLAITGELFF | SEQ ID NO: 267 | TCRBV19-01 | TCRBD02 | TCRBJ02-02 | 0.18 | 3969 |
| CASSQTHPPGELFF | SEQ ID NO: 268 | TCRBV04-03 | | TCRBJ02-02 | 0.17 | 3928 |
| CASSISAGYEQYV | SEQ ID NO: 269 | TCRBV19-01 | TCRBD02 | TCRBJ02-07 | 0.16 | 3684 |
| CASSVDGAYNEQFF | SEQ ID NO: 270 | TCRBV09-01 | TCRBD02 | TCRBJ02-01 | 0.16 | 3650 |
| CAFGVNWDLPHSGNTIYF | SEQ ID NO: 271 | TCRBV30-01 | | TCRBJ01-03 | 0.15 | 3435 |
| CASSFTWGLNTEAFF | SEQ ID NO: 272 | TCRBV12 | | TCRBJ01-01 | 0.14 | 3276 |
| CASSYFSYEQYF | SEQ ID NO: 273 | TCRBV06 | | TCRBJ02-04 | 0.14 | 3150 |
| CASSSDRGLPSGNTIYF | SEQ ID NO: 274 | TCRBV28-01 | TCRBD01 | TCRBJ01-03 | 0.13 | 2973 |
| CSAHEGLEQYF | SEQ ID NO: 275 | TCRBV20-01 | | TCRBJ02-07 | 0.13 | 2906 |
| CASSASWTDYYGYTF | SEQ ID NO: 276 | TCRBV27-01 | TCRBD01 | TCRBJ01-02 | 0.13 | 2902 |
| CASSTGTGSYEQYF | SEQ ID NO: 277 | TCRBV06 | | TCRBJ02-07 | 0.12 | 2718 |
| CASSLWYNQPQHF | SEQ ID NO: 278 | TCRBV27-01 | | TCRBJ01-05 | 0.12 | 2715 |
| CASSPLAAPGSFETQYF | SEQ ID NO: 279 | TCRBV06 | TCRBD02 | TCRBJ02-05 | 0.11 | 2420 |
| CASSVDGDYNEQFF | SEQ ID NO: 280 | TCRBV09-01 | TCRBD02 | TCRBJ02-01 | 0.11 | 2406 |
| CASSPTPSGLWWELFF | SEQ ID NO: 281 | TCRBV12 | TCRBD02 | TCRBJ02-02 | 0.11 | 2400 |
| CASSTGTGLNTEAFF | SEQ ID NO: 282 | TCRBV02-01 | TCRBD01 | TCRBJ01-01 | 0.10 | 2348 |
| CATSALPGQETTDTQYF | SEQ ID NO: 283 | TCRBV24 | TCRBD01 | TCRBJ02-03 | 0.10 | 2267 |
| CASSLVGGLSNQPQHF | SEQ ID NO: 284 | TCRBV27-01 | TCRBD02 | TCRBJ01-05 | 0.10 | 2265 |

TABLE 9
Reference TCRB CDR3 library from dominant SEC24A P469L expanded CD8+ T cells
(MEL38)

| CDR3 amino acid sequence | SEQ ID No. | TCRBV | TCRBD | TCRBJ | Frequency | Read Counts |
|---|---|---|---|---|---|---|
| CASSQQAGGITYNEQFF | SEQ ID NO: 285 | TCRBV03 | TCRBD01 | TCRBJ02-01 | 13.04 | 142392 |
| CASSYSTAGQPQHF | SEQ ID NO: 286 | TCRBV06-05 | TCRBD01 | TCRBJ01-05 | 6.25 | 68241 |
| CASSPTGAGYEQYF | SEQ ID NO: 287 | TCRBV06-05 | TCRBD01 | TCRBJ02-07 | 3.96 | 43243 |
| CASSLLSGSTEAFF | SEQ ID NO: 288 | TCRBV28-01 | TCRBD02 | TCRBJ01-01 | 3.83 | 41830 |
| CASSYGTSTNEQFF | SEQ ID NO: 289 | TCRBV06-05 | TCRBD02 | TCRBJ02-01 | 3.26 | 35641 |
| CASSQGDSGTDTQYF | SEQ ID NO: 290 | TCRBV03 | TCRBD01 | TCRBJ02-03 | 1.57 | 17192 |
| CASSFSNQPQHF | SEQ ID NO: 291 | TCRBV28-01 | | TCRBJ01-05 | 1.57 | 17171 |
| CASSGGQGTQPQHF | SEQ ID NO: 292 | TCRBV28-01 | | TCRBJ01-05 | 1.49 | 16310 |
| CASSYSGAGQPQHF | SEQ ID NO: 293 | TCRBV06-05 | TCRBD01 | TCRBJ01-05 | 1.42 | 15495 |
| CASSLLQGAESPLHF | SEQ ID NO: 294 | TCRBV13-01 | TCRBD01 | TCRBJ01-06 | 1.39 | 15226 |
| CASSPQDRGPNYGYTF | SEQ ID NO: 295 | TCRBV28-01 | TCRBD01 | TCRBJ01-02 | 1.21 | 13219 |
| CASSFDYSYEQYF | SEQ ID NO: 296 | TCRBV05-04 | TCRBD02 | TCRBJ02-07 | 0.88 | 9558 |
| CAAGGVNQPQHF | SEQ ID NO: 297 | TCRBV28-01 | | TCRBJ01-05 | 0.84 | 9144 |
| CASSLLAGELFF | SEQ ID NO: 298 | TCRBV06-05 | TCRBD02 | TCRBJ02-02 | 0.76 | 8282 |
| CASSPSSPYEQYF | SEQ ID NO: 299 | TCRBV12 | TCRBD02 | TCRBJ02-07 | 0.72 | 7894 |
| CASSEGTDTQYF | SEQ ID NO: 300 | TCRBV10-02 | | TCRBJ02-03 | 0.67 | 7299 |
| CASGISNQPQHF | SEQ ID NO: 301 | TCRBV28-01 | | TCRBJ01-05 | 0.66 | 7225 |
| CASSLDPPFDRQNYGYTF | SEQ ID NO: 302 | TCRBV28-01 | TCRBD01 | TCRBJ01-02 | 0.59 | 6456 |
| CASSYGDMAYNEQFF | SEQ ID NO: 303 | TCRBV06-05 | | TCRBJ02-01 | 0.59 | 6440 |
| CATMGTGGSLYYGYTF | SEQ ID NO: 304 | TCRBV28-01 | TCRBD01 | TCRBJ01-02 | 0.59 | 6433 |
| CASSVSNQPQHF | SEQ ID NO: 305 | TCRBV28-01 | | TCRBJ01-05 | 0.58 | 6305 |
| CASSFTSGGYNEQFF | SEQ ID NO: 306 | TCRBV28-01 | TCRBD02 | TCRBJ02-01 | 0.55 | 6055 |
| CASSLYRANTGELFF | SEQ ID NO: 307 | TCRBV28-01 | TCRBD01 | TCRBJ02-02 | 0.53 | 5747 |
| CASSLTSLTDTQYF | SEQ ID NO: 308 | TCRBV06-05 | TCRBD02 | TCRBJ02-03 | 0.51 | 5617 |
| CASSKSKGSPLHF | SEQ ID NO: 309 | TCRBV21-01 | | TCRBJ01-06 | 0.42 | 4580 |
| CASSLAGQGPNSPLHF | SEQ ID NO: 310 | TCRBV05-06 | TCRBD01 | TCRBJ01-06 | 0.41 | 4470 |
| CASSPTGAGQPQHF | SEQ ID NO: 311 | TCRBV06-05 | TCRBD01 | TCRBJ01-05 | 0.40 | 4417 |
| CASSSGTSGSDTQYF | SEQ ID NO: 312 | TCRBV28-01 | TCRBD02 | TCRBJ02-03 | 0.35 | 3791 |
| CASSFSGPRSPQHF | SEQ ID NO: 313 | TCRBV12 | | TCRBJ01-05 | 0.33 | 3592 |
| CASNLQGLDYEQYF | SEQ ID NO: 314 | TCRBV12 | TCRBD01 | TCRBJ02-07 | 0.32 | 3519 |
| CASSLGQGNQPQHF | SEQ ID NO: 315 | TCRBV28-01 | TCRBD01 | TCRBJ01-05 | 0.32 | 3486 |
| CASSFWGANEKLFF | SEQ ID NO: 316 | TCRBV28-01 | TCRBD02 | TCRBJ01-04 | 0.32 | 3474 |
| CASSYSVGVNTEAFF | SEQ ID NO: 317 | TCRBV06-05 | TCRBD02 | TCRBJ01-01 | 0.31 | 3419 |
| CASRYRAAPNQPQHF | SEQ ID NO: 318 | TCRBV28-01 | TCRBD01 | TCRBJ01-05 | 0.30 | 3235 |
| CASSQDAGGVFGNTIYF | SEQ ID NO: 319 | TCRBV03 | TCRBD02 | TCRBJ01-03 | 0.27 | 2894 |
| CASSLYSNQPQHF | SEQ ID NO: 320 | TCRBV28-01 | | TCRBJ01-05 | 0.25 | 2744 |
| CATAPINSPLHF | SEQ ID NO: 321 | TCRBV28-01 | TCRBD02 | TCRBJ01-06 | 0.24 | 2636 |
| CASSPPNQPQHF | SEQ ID NO: 322 | TCRBV28-01 | | TCRBJ01-05 | 0.21 | 2262 |
| CASSFNNQPQHF | SEQ ID NO: 323 | TCRBV28-01 | TCRBD02 | TCRBJ01-05 | 0.21 | 2255 |
| CASGVSNQPQHF | SEQ ID NO: 324 | TCRBV28-01 | TCRBD01 | TCRBJ01-05 | 0.20 | 2180 |
| CASSYESNYGYTF | SEQ ID NO: 325 | TCRBV06 | TCRBD02 | TCRBJ01-02 | 0.19 | 2093 |
| CASSLDVATNEKLFF | SEQ ID NO: 326 | TCRBV06-05 | | TCRBJ01-04 | 0.18 | 2018 |
| CSDSSTGGAGFTF | SEQ ID NO: 327 | TCRBV29-01 | TCRBD01 | TCRBJ01-02 | 0.17 | 1868 |
| CASSESGGGYRWTEAFF | SEQ ID NO: 328 | TCRBV10-01 | TCRBD02 | TCRBJ01-01 | 0.17 | 1839 |
| CASSEGPSGYTF | SEQ ID NO: 329 | TCRBV09-01 | | TCRBJ01-02 | 0.17 | 1838 |
| CASSPGLGEQYF | SEQ ID NO: 330 | TCRBV28-01 | TCRBD02 | TCRBJ02-07 | 0.16 | 1777 |
| CASSLEGVYGYTF | SEQ ID NO: 331 | TCRBV06 | | TCRBJ01-02 | 0.16 | 1758 |
| CASTIGPGITDTQYF | SEQ ID NO: 332 | TCRBV05-06 | | TCRBJ02-03 | 0.16 | 1715 |
| CASSPRDRGPRSPQHF | SEQ ID NO: 333 | TCRBV28-01 | TCRBD01 | TCRBJ01-05 | 0.16 | 1714 |
| CASSRTGAGEKLFF | SEQ ID NO: 334 | TCRBV06-05 | TCRBD01 | TCRBJ01-04 | 0.16 | 1705 |
| CASSLGIAGPYNEQFF | SEQ ID NO: 335 | TCRBV07-06 | TCRBD02 | TCRBJ02-01 | 0.15 | 1634 |
| CAGGLLNQPQHF | SEQ ID NO: 336 | TCRBV28-01 | TCRBD02 | TCRBJ01-05 | 0.14 | 1520 |
| CASSLGQGAQPQHF | SEQ ID NO: 337 | TCRBV28-01 | TCRBD01 | TCRBJ01-05 | 0.14 | 1497 |
| CASSPMNTEAFF | SEQ ID NO: 338 | TCRBV28-01 | TCRBD02 | TCRBJ01-01 | 0.14 | 1493 |
| CASSLSSHGYTF | SEQ ID NO: 339 | TCRBV28-01 | TCRBD02 | TCRBJ01-02 | 0.13 | 1397 |
| CASSFATVGEKLFF | SEQ ID NO: 340 | TCRBV06-05 | TCRBD01 | TCRBJ01-04 | 0.12 | 1364 |
| CASTLYTGDNEQFF | SEQ ID NO: 341 | TCRBV06-05 | TCRBD02 | TCRBJ02-01 | 0.12 | 1358 |
| CASSYSAGGYYGYTF | SEQ ID NO: 342 | TCRBV06-05 | TCRBD01 | TCRBJ01-02 | 0.12 | 1310 |
| CASSYQQGSQPQHF | SEQ ID NO: 343 | TCRBV28-01 | TCRBD01 | TCRBJ01-05 | 0.11 | 1212 |
| CASSPLNTEAFF | SEQ ID NO: 344 | TCRBV19-01 | | TCRBJ01-01 | 0.11 | 1198 |
| CASSWSNQPQHF | SEQ ID NO: 345 | TCRBV28-01 | | TCRBJ01-05 | 0.10 | 1072 |

TABLE 10
Reference TCRB CDR3 library from subdominant AKAP13 Q285K expanded CD8+ T cells
(MEL38)

| CDR3 amino acid sequence | SEQ ID No. | TCRBV | TCRBD | TCRBJ | Frequency | Read Counts |
|---|---|---|---|---|---|---|
| CASSPVTGGDNSPLHF | SEQ ID NO: 346 | TCRBV13-01 | TCRBD01 | TCRBJ01-06 | 8.80 | 69934 |
| CASSSGNYEQYF | SEQ ID NO: 347 | TCRBV13-01 | | TCRBJ02-07 | 8.52 | 67687 |
| CASSLGLSGAYNEQFF | SEQ ID NO: 348 | TCRBV13-01 | TCRBD01 | TCRBJ02-01 | 7.87 | 62566 |
| CAWSVASGNEQFF | SEQ ID NO: 349 | TCRBV30-01 | TCRBD02 | TCRBJ02-01 | 6.44 | 51166 |
| CASSWGQGGYEQYF | SEQ ID NO: 350 | TCRBV13-01 | TCRBD01 | TCRBJ02-07 | 4.66 | 37068 |
| CAWSVGVSNQPQHF | SEQ ID NO: 351 | TCRBV30-01 | | TCRBJ01-05 | 4.36 | 34646 |
| CASSLGQGGELFF | SEQ ID NO: 352 | TCRBV13-01 | TCRBD01 | TCRBJ02-02 | 4.30 | 34205 |
| CASSLGNYEQYF | SEQ ID NO: 353 | TCRBV13-01 | TCRBD01 | TCRBJ02-07 | 2.10 | 16658 |
| CAWSAGTGGNEKLFF | SEQ ID NO: 354 | TCRBV30-01 | TCRBD01 | TCRBJ01-04 | 1.82 | 14434 |
| CAWSVAGGHEQYF | SEQ ID NO: 355 | TCRBV30-01 | TCRBD01 | TCRBJ02-07 | 1.49 | 11869 |
| CASSLGQGYEQYF | SEQ ID NO: 356 | TCRBV13-01 | TCRBD01 | TCRBJ02-07 | 0.98 | 7807 |
| CASSFGQRETEAFF | SEQ ID NO: 357 | TCRBV05-06 | | TCRBJ01-01 | 0.86 | 6805 |
| CASSQGTGVTEAFF | SEQ ID NO: 358 | TCRBV13-01 | TCRBD01 | TCRBJ01-01 | 0.85 | 6761 |
| CASSFGTGYEQYF | SEQ ID NO: 359 | TCRBV06-05 | TCRBD01 | TCRBJ02-07 | 0.81 | 6446 |
| CASSLNPDTQYF | SEQ ID NO: 360 | TCRBV05-06 | | TCRBJ02-03 | 0.33 | 2657 |
| CAWSPGQGGTNEKLFF | SEQ ID NO: 361 | TCRBV30-01 | TCRBD01 | TCRBJ01-04 | 0.29 | 2319 |
| CAWSAYGGELFF | SEQ ID NO: 362 | TCRBV30-01 | TCRBD01 | TCRBJ02-02 | 0.23 | 1846 |
| CAWSVGAGVGEQYF | SEQ ID NO: 363 | TCRBV30-01 | TCRBD02 | TCRBJ02-07 | 0.20 | 1625 |
| CAWSGDRPLAFF | SEQ ID NO: 364 | TCRBV30-01 | | TCRBJ01-01 | 0.18 | 1470 |

TABLE 11
Reference TCRB CDR3 library from domiant EXOC8 Q656P and subdominant PABPC1 R520Q
expanded CD8+ T cells (MEL 218)

| | | | | | | |
|---|---|---|---|---|---|---|
| CDR3 amino acid sequence | SEQ ID No. | TCRBV | TCRBD | TCRBJ | Frequency | Read Counts |

EXOC8 Q656P

| | | | | | | |
|---|---|---|---|---|---|---|
| CASSVGLSETTALYNEQFF | SEQ ID NO: 365 | TCRBV25 | TCRBD02 | TCRBJ02-01 | 4.85 | 15597 |
| CASSLEVVQETQYF | SEQ ID NO: 366 | TCRBV11-02 | | TCRBJ02-05 | 3.64 | 11717 |
| CSARDPASWGEKLFF | SEQ ID NO: 367 | TCRBV20 | | TCRBJ01-04 | 2.75 | 8846 |
| CASSVAGLQGAEQYF | SEQ ID NO: 368 | TCRBV09-01 | | TCRBJ02-07 | 2.5 | 8039 |
| CASSYEQGSYEQYF | SEQ ID NO: 369 | TCRBV06-05 | TCRBD01 | TCRBJ02-07 | 1.87 | 6014 |
| CASSFGPLGMNWAEAFF | SEQ ID NO: 370 | TCRBV06 | | TCRBJ01-01 | 1.53 | 4914 |
| CASSYLSVQETQYF | SEQ ID NO: 371 | TCRBV11-02 | TCRBD02 | TCRBJ02-05 | 0.33 | 1061 |
| CASSLETGYGEQYF | SEQ ID NO: 372 | TCRBV05-05 | TCRBD01 | TCRBJ02-07 | 0.33 | 1062 |
| CASSVFGLAGAEQYF | SEQ ID NO: 373 | TCRBV09-01 | TCRBD02 | TCRBJ02-07 | 0.32 | 1033 |
| CASSEFGGGSPDTQYF | SEQ ID NO: 374 | TCRBV09-01 | TCRBD02 | TCRBJ02-03 | 0.21 | 661 |
| CASSVYGGAEAFF | SEQ ID NO: 375 | TCRBV09-01 | TCRBD02 | TCRBJ01-01 | 0.12 | 370 |
| CASSTYGLAGETQYF | SEQ ID NO: 376 | TCRBV09-01 | TCRBD02 | TCRBJ02-05 | 0.1 | 322 |

PABPC1 R520Q

| | | | | | | |
|---|---|---|---|---|---|---|
| CSVENRVIYGYTF | SEQ ID NO: 377 | TCRBV29-01 | TCRBD01 | TCRBJ01-02 | 16.65 | 28165 |
| CSVEDPTFYGYTF | SEQ ID NO: 378 | TCRBV29-01 | | TCRBJ01-02 | 15.13 | 25599 |
| CASSLGSSGNTIYF | SEQ ID NO: 379 | TCRBV09-01 | | TCRBJ01-03 | 9.83 | 16628 |
| CSVEGQIAGKYGYTF | SEQ ID NO: 380 | TCRBV29-01 | | TCRBJ01-02 | 8.42 | 14240 |
| CASSYGTSGTEQFF | SEQ ID NO: 381 | TCRBV07-06 | TCRBD02 | TCRBJ02-01 | 3.20 | 5412 |
| CSVEDGAAKQIYGYTF | SEQ ID NO: 382 | TCRBV29-01 | | TCRBJ01-02 | 0.47 | 797 |
| CASSVEYSNQPQHF | SEQ ID NO: 383 | TCRBV02-01 | TCRBD02 | TCRBJ01-05 | .27 | 457 |
| CSVEDRVNYGYTF | SEQ ID NO: 384 | TCRBV29-01 | TCRBD01 | TCRBJ01-02 | 0.16 | 275 |
| CASSQWSSTNEKLFF | SEQ ID NO: 385 | TCRBV14-01 | | TCRBJ01-04 | 0.12 | 199 |
| CARNHDRDRLYEQYF | SEQ ID NO: 386 | TCRBV02-01 | TCRBD01 | TCRBJ02-07 | 0.11 | 185 |
| CASSSWGTSDEQYF | SEQ ID NO: 387 | TCRBV07-09 | TCRBD02 | TCRBJ02-07 | 0.10 | 172 |

TABLE 12
MEL69 HLA A2

| | | | | | | Predicted | | |
|---|---|---|---|---|---|---|---|---|
| | | | | | | Affinity (nM) | | |
| | | | | | | | | Amino |
| | | | | | | | | Acid |
| | | | | | | | | Substi- |
| | Hugo | AAS- | | wild-type | | | wild- | tution |
| CHR | Symbol | peptide | AAS-SEQID | peptide | WT SEQ ID | mutated | type | (AAS) |
| 2 | MPV17 | VLDGFIPGT | SEQ ID NO: 127 | VLDRFIPGT | SEQ ID NO: 128 | 51 | 233 | R75G |
| 5 | RUFY1 | KLADYLNVL | SEQ ID NO: 129 | KLADYLKVL | SEQ ID NO: 130 | 5 | 15 | K225N |
| 7 | LANCL2 | YSFLFLYRL | SEQ ID NO: 131 | YSFLSLYRL | SEQ ID NO: 132 | 71 | 213 | S370F |
| 12 | UBE3B | HLGFLSPRV | SEQ ID NO: 133 | HLGSLSPRV | SEQ ID NO: 134 | 60 | 42 | S321F |
| 16 | AARS | RVVFIGVPV | SEQ ID NO: 136 | RVVSAGVPV | SEQ ID NO: 136 | 488 | 237 | S698F |
| 17 | CASC3 | SMSPGQPPL | SEQ ID NO: 137 | SMSPGQPPP | SEQ ID NO: 138 | 17 | 8696 | P513L |
| X | ZMYM3 | VVDFTESIPV | SEQ ID NO: 139 | VVDSTESIPV | SEQ ID NO: 140 | 444 | 360 | S258F |
| 2 | GPC1 | RLFGEAPREIL | SEQ ID NO: 141 | RPFGEAPREL | SEQ ID NO: 142 | 83 | 21700 | P201L |
| 1 | SRSF11 | ALAALGLSGA | SEQ ID NO: 143 | ALAALGLPGA | SEQ ID NO: 144 | 176 | 73 | P137S |
| 12 | OASL | TIPSEIQIFV | SEQ ID NO: 145 | TIPSEIQVFV | SEQ ID NO: 146 | 274 | 470 | V438I |
| 19 | SIPA1L3 | ILGIFNEFV | SEQ ID NO: 147 | ILGISNEFV | SEQ ID NO: 148 | 45 | 118 | S893F |
| 18 | NPC1 | FVGALSFSI | SEQ ID NO: 149 | FVGVLSFSI | SEQ ID NO: 150 | 23 | 88 | V845A |
| 10 | MARCH5 | YYLDLANRL | SEQ ID NO: 151 | YVLDLADRL | SEQ ID NO: 152 | 37 | 54 | D90N |
| 11 | SCYL1 | FLFELIPEP | SEQ ID NO: 153 | FPFELIPEP | SEQ ID NO: 154 | 21 | 12401 | P13L |
| 5 | PRRC1 | QMIYSAARV | SEQ ID NO: 155 | QMIYSAARA | SEQ ID NO: 156 | 79 | 1783 | A431V |
| 13 | LMO7 | SLVEEQSPA | SEQ ID NO: 157 | SPVEEQSPA | SEQ ID NO: 158 | 79 | 21881 | P583L |
| 19 | HSD11B1L | MAFPEAPESV | SEQ ID NO: 159 | MASPEAPESV | SEQ ID NO: 160 | 156 | 1145 | S90F |
| 19 | PPAN | SLVRDVFSSL | SEQ ID NO: 161 | SLVRDVVSSL | SEQ ID NO: 162 | 106 | 135 | V69F |
| 7 | BRAF | LATEKSRWS | SEQ ID NO: 163 | LATVKSRWS | SEQ ID NO: 164 | 24853 | 27478 | V600E |
| | | MEL69A.2 | MEL69A.2 | MEL69A.2 | | MEL69B.2 | MEL69B.2 | |
| | Hugo | (Limb) | (Limb) | (Limb) | MEL69B.2 (Scalp) | Scalp) RNA | (Scalp) | |
| CHR | Symbol | Exome VAF | RNA VAF | FPKM | Exome Tumor VAF | Tumor VAF | FPKM | |
| 2 | MPV17 | 34.78 | 31.51 | 44.1711 | 36.59 | 37.87 | 44.5254 | |
| 5 | RUFY1 | 32.5 | 17.95 | 10.8626 | 23.81 | 42.05 | 12.321 | |
| 7 | LANCL2 | 16.07 | 31.86 | 15.3511 | 31.58 | 42.57 | 15.187 | |
| 12 | UBE3B | 28.57 | 41.94 | 13.1866 | 37.68 | 42.11 | 18.9171 | |
| 16 | AARS | 13.51 | 43.51 | 21.7187 | 39.02 | 48.85 | 44.5936 | |
| 17 | CASC3 | 21.05 | 26.79 | 6.77417 | 33.33 | 28.81 | 8.93879 | |
| X | ZMYM3 | 35.29 | 51.81 | 9.72465 | 80.95 | 75.44 | 14.715 | |
| 2 | GPC1 | 28.12 | 30 | 7.40362 | 33.33 | 38.89 | 9.89646 | |
| 1 | SRSF11 | 11.76 | 26.4 | 63.5826 | 46.15 | 44.17 | 62.4002 | |
| 12 | OASL | 16.36 | 14.79 | 10.8827 | 40.43 | 27.56 | 9.78642 | |
| 19 | SIPA1L3 | 8.33 | 29.41 | 1.41955 | 30 | 64.71 | 3.27408 | |
| 18 | NPC1 | 30.77 | 32 | 32.9957 | 46.67 | 48.27 | 48.3298 | |
| 10 | MARCH5 | 0 | 0 | 9.44984 | 30.43 | 37.8 | 11.4002 | |
| 11 | SCYL1 | 15.38 | 27.54 | 29.3756 | 46.15 | 37.41 | 48.8269 | |
| 5 | P44C1 | 11.11 | 26.14 | 26.921 | 30.56 | 36.17 | 31.9828 | |
| 13 | LMO7 | 23.68 | 0 | 12.5597 | 30.25 | 13.04 | 8.01764 | |
| 19 | HSD11B1L | 18.52 | 0 | 0.551889 | 33.33 | 100 | 0.367626 | |
| 19 | PPAN | 0 | 0 | 7.52204 | 34.29 | 43.53 | 11.0531 | |
| 7 | BRAF | 30 | 67.67 | 13.3533 | 56.25 | 56.1 | 14.5002 | |
| | | | | | | Predicted | | |
| | | | | | | Affinity (nM) | | |
| | | | | | | | | Amino |
| | | | | | | | | Acid |
| | | | | | | | | Substi- |
| | Hugo | AAS- | AAS- | wild-type | | | wild- | tution |
| CHR | Symbol | peptide | SEQID | peptide | WT SEQ ID | mutated | type | (AAS) |
| 5 | ZSWIM6 | LSALTRCEK | SEQ ID NO: 388 | LSALTLCEK | SEQ ID NO: 389 | 295 | 215 | L1002R |
| 12 | KIAA0528 | LSACNSPSK | SEQ ID NO: 390 | LPACNSPSK | SEQ ID NO: 391 | 91 | 14975 | P256S |
| 12 | SMARCC2 | KVFEHVGSR | SEQ ID NO: 392 | KVSEHVGSR | SEQ ID NO: 393 | 69 | 390 | S624F |
| 19 | PIP5K1C | FISNTVFRK | SEQ ID NO: 394 | FMSNTVFRK | SEQ ID NO: 395 | 21 | 25 | M439I |
| 20 | PPP1R16B | HQCCIDNFK | SEQ ID NO: 396 | HQCCIDNFE | SEQ ID NO: 397 | 162 | 21019 | E114K |
| 22 | RHBDD3 | SSAAGSFGY | SEQ ID NO: 398 | SSAAGSCGY | SEQ ID NO: 399 | 51 | 668 | C119F |
| X | ERCC6L | KIYRRQIFK | SEQ ID NO: 400 | KIYRRQVFK | SEQ ID NO: 401 | 12 | 13 | V476I |
| 7 | BRAF | LATEKSRWS | SEQ ID NO: 163 | LATVKSRWS | SEQ ID NO: 164 | 24853 | 27478 | V600E |

| | | MEL69A.2 | MEL69A.2 | MEL69A.2 | | MEL69B.2 | MEL69B.2 |
|---|---|---|---|---|---|---|---|
| | Hugo | (Limb) | (Limb) | (Limb) | MEL69B.2 (Scalp) | (Scalp) RNA | (Scalp) |
| CHR | Symbol | Exome VAF | RNA VAF | FPKM | Exome Tumor VAF | Tumor VAF | FPKM |
| 5 | ZSWIM6 | 25.49 | 43.75 | 9.3725 | 33.33 | 51.16 | 11.045 |
| 12 | KIAA0528 | 28.57 | 11.96 | 24.255 | 50 | 25 | 20.069 |
| 12 | SMARCC2 | 27.66 | 17.78 | 14.734 | 26.83 | 41.77 | 20.227 |
| 19 | PIP5K1C | 22.5 | 23.81 | 6.1374 | 24 | 38.57 | 11.467 |
| 20 | PPP1R16B | 18.92 | 15.79 | 2.8959 | 25.81 | 45.16 | 2.8599 |
| 22 | RHBDD3 | 30 | 57.14 | 11.48 | 66.67 | 83.33 | 8.2471 |
| X | ERCC6L | 55.56 | 69.23 | 2.4877 | 43.24 | 63.64 | 2.4041 |
| 7 | BRAF | 30 | 67.67 | 13.353 | 56.25 | 56.1 | 14.6 |

Predicted affinity (MT and WT score) as determined using NetMCH3.4 algorithm.
VAF = Variant Allelic Fraction as determined from exome sequencing. BRAF VAF are reported as these were used as comparator to assess clonality of other mutations
FPKM = Fragment Per Kilobase of transcript per Million per transcriptome as determined from cDNA-capture data.
BRAF VAF values are reported and were used as comparator to interpret frequencies of remaining missense mutation encoding-genes.
Candidates formulated in vaccine are shown bolded.

TABLE 13
MEL 66 HLA A2

| | | | | | | Predicted | | |
|---|---|---|---|---|---|---|---|---|
| | | | | | | Affinity (nM) | | |
| | | | | | | | | Amino |
| | | | | | | | | Acid |
| | | | | | | | | Substi- |
| | Hugo | AAS- | | wild-type | | | wild- | tution |
| CHR | Symbol | peptide | AAS-SEQID | peptide | WT SEQ ID | mutated | type | (AAS) |
| 7 | LMBR1 | LLLLLCTSV | SEQ ID NO: 402 | LLLLLCTPV | SEQ ID NO: 403 | 19 | 10 | P210S |
| 2 | SH3BP4 | RLIQGFVLL | SEQ ID NO: 404 | RLIQDFVLL | SEQ ID NO: 405 | 41 | 51 | D843G |
| 1 | ATP2B4 | QLIVIFIFV | SEQ ID NO: 406 | QLIVIFILV | SEQ ID NO: 407 | 34 | 60 | L934F |
| 2 | MGAT4A | ALAFITFFL | SEQ ID NO: 408 | ALAFITSFL | SEQ ID NO: 409 | 7 | 26 | S17F |
| X | PORCN | LLHGFSFYL | SEQ ID NO: 410 | LLHGFSFHL | SEQ ID NO: 411 | 5 | 11 | H346Y |
| 7 | PHKG1 | TLFENTPKA | SEQ ID NO: 412 | ALFENTPKA | SEQ ID NO: 413 | 18 | 14 | A401T |
| 14 | ATG2B | KLNLVCCEL | SEQ ID NO: 414 | KLMPVCCEL | SEQ ID NO: 415 | 95 | 23 | P679L |
| 12 | CAMKK2 | YLGMESFIV | SEQ ID NO: 416 | HLGMESFIV | SEQ ID NO: 417 | 9 | 111 | H46Y |
| 2 | ZDBF2 | YILKYSVFL | SEQ ID NO: 418 | YISKYSVFL | SEQ ID NO: 419 | 5 | 11 | S2228L |
| 11 | EXT2 | VLQEATICV | SEQ ID NO: 420 | VLQEATFCV | SEQ ID NO: 421 | 13 | 7 | F350I |
| 9 | ZNF658 | GLYDKAICI | SEQ ID NO: 422 | GLYDKTICI | SEQ ID NO: 423 | 25 | 13 | T228A |
| 14 | PLEKHH1 | YLLKIGSQV | SEQ ID NO: 424 | YLLKMGSQV | SEQ ID NO: 425 | 15 | 18 | M588I |
| 17 | GAS7 | FLGEAWAQV | SEQ ID NO: 426 | SLGEAWAQV | SEQ ID NO: 427 | 11 | 32 | S270F |
| 20 | SLC2A10 | FLSSMACCI | SEQ ID NO: 428 | SLSSMACCI | SEQ ID NO: 429 | 27 | 232 | S113F |
| 3 | LMLN | SLVVTLWPL | SEQ ID NO: 430 | SLVVTLWLL | SEQ ID NO: 431 | 12 | 36 | L637P |
| 2 | CERS6 | SMWRFTFYL | SEQ ID NO: 432 | SMWRFSFYL | SEQ ID NO: 433 | 3 | 3 | S140T |
| 6 | CUL9 | CLLQLCPRL | SEQ ID NO: 434 | RLLQLCPRL | SEQ ID NO: 435 | 64 | 25 | R1335C |
| 12 | GCN1L1 | SLLRSLENV | SEQ ID NO: 436 | SLLRSPENV | SEQ ID NO: 437 | 21 | 59 | P274L |
| 20 | SLC13A3 | FLISILYSA | SEQ ID NO: 438 | FLISIPYSA | SEQ ID NO: 439 | 3 | 4 | P239L |
| 8 | ARHGEF10 | YLLRWSVPL | SEQ ID NO: 440 | YLLKWSVPL | SEQ ID NO: 441 | 3 | 3 | K697R |
| 22 | SF3A1 | MLTTAIPKV | SEQ ID NO: 442 | MPTTAIPKV | SEQ ID NO: 443 | 5 | 12945 | P6L |
| 1 | WDR63 | HILEILWTL | SEQ ID NO: 444 | HILEIPWTL | SEQ ID NO: 445 | 7 | 11 | P793L |
| 14 | SLC24A4 | NMFDILVGL | SEQ ID NO: 446 | NVFDILVGL | SEQ ID NO: 447 | 6 | 53 | V527M |
| 6 | PDE7B | RMWDFDIFL | SEQ ID NO: 448 | GMWDFDIFL | SEQ ID NO: 449 | 3 | 3 | G113R |
| 1 | RASAL2 | IMSSSLFNL | SEQ ID NO: 450 | IMSPSLFNL | SEQ ID NO: 451 | 6 | 8 | P637S |
| 7 | AKAP9 | RLSDFSEQL | SEQ ID NO: 452 | RLSDLSEQL | SEQ ID NO: 453 | 30 | 52 | L974F |
| | Hugo | MEL66A | MEL66A | MEL66A | MEL66D Exome | MEL66D RNA | MEL66D | |
| CHR | Symbol | Exome VAF | RNA VAF | FPKM | VAF | VAF | FPKM | |
| 7 | LMBR1 | 66.07 | 95.59 | 133.906 | 31.17 | 64.38 | 32.8169 | |
| 2 | SH3BP4 | 51.72 | 38.56 | 24.5197 | 29.41 | 41.53 | 27.9068 | |
| 1 | ATP2B4 | 48.48 | 36.47 | 37.9108 | 25.81 | 35.89 | 36.7154 | |
| 2 | MGAT4A | 48 | 17.12 | 34.4185 | 23.08 | 7.38 | 61.5058 | |
| X | PORCN | 47.37 | 89.68 | 22.2618 | 8.86 | 78.2 | 17.7896 | |
| 7 | PHKG1 | 47.06 | 52.94 | 1.77883 | 17.86 | 34.78 | 1.61569 | |
| 14 | ATG2B | 46.15 | 36.41 | 40.641 | 17.14 | 37.26 | 38.7526 | |
| 12 | CAMKK2 | 43.59 | 47.62 | 15.4478 | 14.89 | 19.78 | 14.1399 | |
| 2 | ZDBF2 | 42.22 | 89.47 | 7.94103 | 14.74 | 53.97 | 11.8555 | |
| 11 | EXT2 | 42 | 38.9 | 53.8156 | 10 | 40.85 | 37.7597 | |
| 9 | ZNF658 | 40.37 | 48.09 | 17.1165 | 20.83 | 33.77 | 13.9748 | |
| 14 | PLEKHH1 | 40 | 50.88 | 14.6035 | 46.67 | 41.96 | 25.1339 | |
| 17 | GAS7 | 38.48 | 19.74 | 10.3323 | 31.82 | 26.24 | 31.4939 | |
| 20 | SLC2A10 | 36.59 | 46.15 | 1.86998 | 21.43 | 63.33 | 2.29521 | |
| 3 | LMLN | 36.17 | 45.45 | 7.56894 | 25.93 | 52.17 | 6.56604 | |
| 2 | CERS6 | 36.11 | 42.02 | 10.198 | 14.81 | 33.53 | 7.74818 | |
| 6 | CUL9 | 36 | 38.6 | 7.63523 | 22.58 | 26.88 | 13.4072 | |
| 12 | GDN1L1 | 34.78 | 33.67 | 38.6382 | 19.15 | 28.24 | 45.0198 | |
| 20 | SLC13A3 | 34 | 59.26 | 4.30641 | 15.94 | 62.79 | 5.58358 | |
| 8 | ARHGEF10 | 33.33 | 43.24 | 13.6682 | 19.57 | 35.42 | 14.6281 | |
| 22 | SF3A1 | 32.56 | 37.95 | 32.8032 | 15.58 | 32.72 | 56.3619 | |
| 1 | WDR63 | 31.82 | 46.82 | 41.4768 | 11.94 | 36.11 | 3.23577 | |
| 14 | SLC24A4 | 29.82 | 53.04 | 72.1497 | 11.54 | 56.82 | 3.81134 | |
| 6 | PDE7B | 26.91 | 32.69 | 6.92805 | 14.29 | 34.88 | 6.604 | |
| 1 | RASAL2 | 33.33 | 31.23 | 21.9958 | 16.07 | 38.83 | 26.2991 | |
| 7 | AKAP9 | 71.05 | 86.04 | 60.8703 | 26.56 | 26.56 | 26.56 | |
| | | | | | | Predicted | | |
| | | | | | | Affinity (nM) | | |
| | | | | | | | | Amino |
| | | | | | | | | Acid |
| | | | | | | | | Substi- |
| | Hugo | AAS- | | wild-type | | | wild- | tution |
| CHR | Symbol | peptide | AAS-SEQID | peptide | WT SEQ ID | mutated | type | (AAS) |
| 14 | AHNAK2 | MPKFKMSSF | SEQ ID NO: 454 | MPKFKMPSF | SEQ ID NO: 455 | 9 | 14 | P3151S |
| 4 | DDX60 | LPSMHRHQI | SEQ ID NO: 456 | LPSMYRHQI | SEQ ID NO: 457 | 35 | 90 | Y194H |
| 19 | TLE2 | LPRAKKLIL | SEQ ID NO: 458 | LPRAKELIL | SEQ ID NO: 459 | 14 | 40 | E288K |
| 9 | DMRTA1 | FSNYRRSRL | SEQ ID NO: 460 | FPNYRRSRL | SEQ ID NO: 461 | 80 | 14 | P338S |
| 3 | WDR52 | QLILRTKAF | SEQ ID NO: 462 | QPILRTKAF | SEQ ID NO: 463 | 38 | 41 | P264L |
| 7 | FKBP3 | YLKYHCNAS | SEQ ID NO: 464 | YLKYHYNAS | SEQ ID NO: 465 | 62 | 32 | Y449C |
| 18 | SOCS6 | SLRSHHYSL | SEQ ID NO: 466 | SLRSHHYSP | SEQ ID NO: 467 | 6 | 75 | P134L |
| 2 | CHPF | FFSMHFQAF | SEQ ID NO: 468 | FFPMHFQAF | SEQ ID NO: 469 | 20 | 49 | P641S |
| 2 | DUSP2 | LFRYKSISV | SEQ ID NO: 470 | LFRYKSIPV | SEQ ID NO: 471 | 95 | 120 | P223S |
| 1 | LRRC42 | NLRYFAKSL | SEQ ID NO: 472 | NLRYSAKSL | SEQ ID NO: 473 | 26 | 40 | S85F |
| 7 | BRAF | LATEKSRWS | SEQ ID NO: 163 | LATVKSRWS | SEQ ID NO: 164 | 24853 | 27478 | V600E |

| | | MEL66A | MEL66A | | MEL66D | MEL66D | |
|---|---|---|---|---|---|---|---|
| | Hugo | Exome | RNA | MEL66A | Exome | RNA | MEL66D |
| CHR | Symbol | VAF | VAF | FPKM | VAF | VAF | FPKM |
| 14 | AHNAK2 | 74.74 | 95.54 | 14.8985 | 35.24 | 93.66 | 40.6564 |
| 4 | DDX60 | 41.51 | 30.09 | 35.1655 | 28.26 | 24.84 | 72.2322 |
| 19 | TLE2 | 42 | 38.6 | 4.18558 | 27.59 | 42.86 | 2.88573 |
| 9 | DMRTA1 | 31.25 | 29.61 | 16.3335 | 24.19 | 35.14 | 2.76312 |
| 3 | WDR52 | 40 | 48.95 | 28.3206 | 22.22 | 26.32 | 12.81 |
| 7 | FKBP3 | 40.19 | 45.63 | 210.808 | 19.42 | 44.71 | 167.962 |
| 18 | SOCS6 | 39.13 | 27.48 | 30.9938 | 16.67 | 27.97 | 23.4984 |
| 2 | CHPF | 40 | 47.62 | 32.2709 | 15.73 | 48.12 | 27.2727 |
| 2 | DUSP2 | 41.98 | 19.78 | 5.98827 | 14.63 | 15.14 | 19.9318 |
| 1 | LRRC42 | 32.53 | 39.61 | 27.2896 | 12.05 | 36.05 | 25.2227 |
| 7 | BRAF | 66.67 | | | 33.33 | | |

1. A method of treating a cancer in a subject in need thereof, comprising:
providing a neoantigen peptide encoded in DNA of a tumor of the subject, wherein the neoantigen peptide consists of from 8 to 13 amino acids, binds in silico to an HLA class I molecule with an affinity of <500 nM and a stability>2 h and binds in vitro to an HLA class I molecule with an affinity of <4.7 log (IC50, nM);
transfecting at least one HLA class I positive cell with at least one tandem minigene construct comprising at least one sequence encoding the at least one neoantigen;
identifying a complex comprising the at least one HLA molecule and the at least ogre neoantigen peptide produced by the at least one HLA class I positive cell;
forming a vaccine comprising the at least one neoantigen; and
administering the vaccine to the subject, wherein at least one tumor cell of the cancer comprises at least one polypeptide comprising at least one amino acid substitution.

2. A method in accordance with claim 1, wherein the at least one neoantigen peptide consists of 9 amino acids.

3. A method in accordance with claim 1, wherein the at least one neoantigen binds in silico to an HLA class I molecule with an affinity of <250 nM.

4. A method in accordance with claim 1, wherein the at least one neoantigen binds in vitro to an HLA class I molecule with an affinity of <3.8 log (IC50, nM).

5. A method in accordance with claim 1, wherein the vaccine comprises at least seven neoantigen peptides.

6. A method in accordance with claim 1, wherein the HLA class I molecule is selected from the group consisting of HLA-A*01:01, HLA-B*07:02, HLA-A*02:01, HLA-B*07:03, HLA-A*02:02, HLA-B*08:01, HLA-A*02:03, HLA-B*15:01, HLA-A*02:05, HLA-B*15:02, HLA-A*02:06, HLA-B*15:03, HLA-A*02:07, HLA-B*15:08, HLA-*03:01, HLA-B*15:12, HLA-A*11:01, HLA-B*15:16, HLA-A*11:02, HLA-B*15:18, HLA-A*24:02, HLA-B*27:03, HLA-A*29:01, HLA-B*27:05, HLA-A*29:02, HLA-B*27:08, HLA-A34:02, HLA-B*35:01, HLA-A*36:01, HLA-B*35:08, HLA-B*42:01, HLA-B*53:01, HLA-B*54:01, HLA-B*56:01, HLA-B*56:02, HLA-B*57:01, HLA-B*57:02, HLA-B*57:03, HLA-B*58:01, HLA-B*67:01 and HLA-B*81:01.

7. A method in accordance with claim 1, wherein the HLA class I molecule is selected from the group consisting of an HLA-A*02:01 molecule, an HLA-A*11:01 molecule and an HLA-B*08:01 molecule.

8. A method in accordance with claim 1, wherein the at least one HLA class I positive cell is at least one HLA class I positive melanoma cell.

9. A method in accordance with claim 1, wherein the cancer is selected from the group consisting of skin cancer, lung cancer, bladder cancer, colorectal cancer, gastrointestinal cancer, esophageal cancer, gastric cancer, intestinal cancer, breast cancer, and a mismatch repair deficiency cancer.

10. A method in accordance with claim 1, wherein the cancer is a melanoma.

11. A method in accordance with claim 1, wherein the forming a vaccine comprises:
providing a culture comprising dendritic cells obtained from the subject; and
contacting the dendritic cells with the at least one neoantigen peptide, thereby forming dendritic cells comprising the at least one neoantigen peptide.

12. A method in accordance with claim 11, further comprising:
administering to the subject the dendritic cells comprising the at least one neoantigen peptide;
obtaining a population of CD8+ T cells from a peripheral blood sample from the subject, wherein the CD8+ cells recognize the at least one neoantigen; and
expanding the population of CD8+ T cells that recognizes the neoantigen.

13. A method in accordance with claim 1, wherein the identifying a complex comprises performing an assay selected from the group consisting of an LC/MS assay, a reverse phase HPLC assay and a combination thereof.

14. A method of treating a cancer in a subject in need thereof, comprising:
a) providing a sample of a tumor from a subject;
b) performing exome sequencing on the sample to identify one or more amino acid substitutions comprised by the tumor exome;
c) performing transcriptome sequencing on the sample to verify expression of the amino acid substitutions identified in b); and
d) selecting at least one candidate neoantigen peptide sequence from amongst the amino acid substitutions identified in c) according to the following criteria:
i) Exome VAF>10%;
ii) Transcription VAF>10%;
iii) Alternate reads>5;
iv) FPKM>1;
v) binds in silico to an HLA class I molecule with an affinity of <500 nM and a stability>2 h;
e) performing an in vitro HLA class I binding assay;
f) selecting at least one candidate neoantigen peptide sequence from amongst the amino acid substitutions identified in d) that bind HLA class one molecules with an affinity of <4.7 log (IC50, nM) in the assay performed in e)
g) transfecting at least one HLA class I positive cell with at least one tandem minigene construct comprising at least one sequence encoding the at least one neoantigen;
h) identifying a complex comprising the at least one HLA molecule and the at least one neoantigen peptide produced by the at least one HLA class I positive cell;
i) forming a vaccine comprising the at least one neoantigen; and
j) administering the vaccine to the subject, wherein at least one tumor cell of the cancer comprises at least one polypeptide comprising the one or more amino acid substitutions.

15. A method in accordance with claim 14, wherein the in vitro HLA class I binding assay is selected from the group consisting of a T2 assay and a fluorescence polarization assay.

16. A method in accordance with claim 14, wherein the forming a vaccine comprises:
providing a culture comprising dendritic cells obtained from the subject; and
contacting the dendritic cells with the at least one neoantigen peptide, thereby forming dendritic cells comprising the at least one neoantigen peptide.

17. A method in accordance with claim 16, further comprising:
administering to the subject the dendritic cells comprising the at least one neoantigen peptide;
obtaining a population of CD8+ T cells from a peripheral blood sample from the subject, wherein the CD8+ T cells recognize the at least one neoantigen; and
expanding the population of CD8+ T cells that recognizes the neoantigen.

18. A method in accordance with claim 14, wherein the identifying a complex comprising the at least one HLA molecule and the at least one neoantigen peptide comprises performing an assay selected from the group consisting of a LC/MS assay, a reverse phase HPLC assay and a combination thereof.

19. A method of treating a cancer in a subject in need thereof, comprising:
providing a neoantigen peptide encoded in DNA of a tumor of the subject, wherein the neoantigen peptide consists of from 8 to 13 amino acids, binds in silico to an HLA class I molecule with an affinity of <500 nM and a stability>2 h;
performing an in vitro HLA class I molecule binding assay to identify at least one neoantigen peptide which binds in vitro to an HLA class I molecule with an affinity of <4.7 log (IC50, nM);
transfecting at least one HLA class I positive cell with at least one tandem minigene construct comprising at least one sequence encoding the at least one neoantigen;
identifying a complex comprising the at least one HLA molecule and the at least one neoantigen peptide produced by the at least one HLA class I positive cell;
forming a vaccine comprising the at least one neoantigen; and
administering the vaccine to the subject, wherein at least one tumor cell of the cancer comprises at least one polypeptide comprising at least one amino acid substitution.

20. A method in accordance with claim 19, wherein the in vitro HLA class I binding assay is selected from the group consisting of a T2 assay and a fluorescence polarization assay.

21. A method in accordance with claim 19, wherein the identifying a complex comprising the at least one HLA molecule and the at least one neoantigen peptide comprises performing an assay selected from the group consisting of an LC/MS assay, a reverse phase HPLC assay and a combination thereof.

22. A method in accordance with claim 19, wherein the forming a vaccine comprises:
providing a culture comprising dendritic cells obtained from the subject; and
contacting the dendritic cells with the at least one neoantigen peptide, thereby forming dendritic cells comprising the at least one neoantigen peptide.

23. A method in accordance with claim 22, further comprising:
administering to the subject the dendritic cells comprising the at least one neoantigen peptide;
obtaining a population of CD8+ T cells from a peripheral blood sample from the subject, wherein the CD8+ cells recognize the at least one neoantigen; and
expanding the population of CD8+ T cells that recognizes the neoantigen.


SEQUENCE LISTING (474 sequences)

SEQ ID NO: 1 - protein, 9 aa, Homo sapiens
KLMNIQQKL

SEQ ID NO: 2 - protein, 9 aa, Homo sapiens
FVSALCMFL

SEQ ID NO: 3 - protein, 9 aa, Homo sapiens
QLCEDASTV

SEQ ID NO: 4 - protein, 9 aa, Homo sapiens
LMSIIIVGV

SEQ ID NO: 5 - protein, 9 aa, Homo sapiens
LIMTCCVAL

SEQ ID NO: 6 - protein, 9 aa, Homo sapiens
ALVSGNQQL

SEQ ID NO: 7 - protein, 9 aa, Homo sapiens
YISKCWDYA

SEQ ID NO: 8 - protein, 9 aa, Homo sapiens
FIMGISILL

SEQ ID NO: 9 - protein, 9 aa, Homo sapiens
IQLSWVLIA

SEQ ID NO: 10 - protein, 9 aa, Homo sapiens
ILMTWNLHS

SEQ ID NO: 11 - protein, 9 aa, Homo sapiens
YIFLENLAL

SEQ ID NO: 12 - protein, 9 aa, Homo sapiens
QLSCISTYV

SEQ ID NO: 13 - protein, 9 aa, Homo sapiens
FLYNLLTRV

SEQ ID NO: 14 - protein, 9 aa, Homo sapiens
WMAPEVILV

SEQ ID NO: 15 - protein, 9 aa, Homo sapiens
QLDKCSAFV

SEQ ID NO: 16 - protein, 9 aa, Homo sapiens
KLMNIQQQL

SEQ ID NO: 17 - protein, 9 aa, Homo sapiens
FVSALRMFL

SEQ ID NO: 18 - protein, 9 aa, Homo sapiens
QLREDASTV

SEQ ID NO: 19 - protein, 9 aa, Homo sapiens
PMSIIIVGV

SEQ ID NO: 20 - protein, 9 aa, Homo sapiens
LIMTCYVAL

SEQ ID NO: 21 - protein, 9 aa, Homo sapiens
APVSGNQQL

SEQ ID NO: 22 - protein, 9 aa, Homo sapiens
YISKCWDHA

SEQ ID NO: 23 - protein, 9 aa, Homo sapiens
FITGISILL

SEQ ID NO: 24 - protein, 9 aa, Homo sapiens
IQLSGVLIA

SEQ ID NO: 25 - protein, 9 aa, Homo sapiens
ILMTGNLHS

SEQ ID NO: 26 - protein, 9 aa, Homo sapiens
YIFLGNLAL

SEQ ID NO: 27 - protein, 9 aa, Homo sapiens
QLSCTSTYV

SEQ ID NO: 28 - protein, 9 aa, Homo sapiens
FLYNPLTRV

SEQ ID NO: 29 - protein, 9 aa, Homo sapiens
WMAPEVILA

SEQ ID NO: 30 - protein, 9 aa, Homo sapiens
QLDQCSAFV

SEQ ID NO: 31 - protein, 9 aa, Homo sapiens
SVGGVFTSV

SEQ ID NO: 32 - protein, 10 aa, Rattus norvegicus
SVVSTDDDLA

SEQ ID NO: 33 - protein, 9 aa, West Nile virus
SVGGVFTSV

SEQ ID NO: 34 - protein, 20 aa, Homo sapiens
TGPIFKLMNIQQKLMKTNLN

SEQ ID NO: 35 - protein, 9 aa, Homo sapiens
NLSGNTALL

SEQ ID NO: 36 - protein, 9 aa, Homo sapiens
DLSGNTALL

SEQ ID NO: 37 - protein, 9 aa, Homo sapiens
QTIDNIVFL

SEQ ID NO: 38 - protein, 9 aa, Homo sapiens
QTIDNIVFF

SEQ ID NO: 39 - protein, 9 aa, Homo sapiens
KMIGNHLWV

SEQ ID NO: 40 - protein, 9 aa, Homo sapiens
EMIGNHLWV

SEQ ID NO: 41 - protein, 9 aa, Homo sapiens
FTMLALQDL

SEQ ID NO: 42 - protein, 9 aa, Homo sapiens
FTMLALRDL

SEQ ID NO: 43 - protein, 9 aa, Homo sapiens
SLWNAIDFF

SEQ ID NO: 44 - protein, 9 aa, Homo sapiens
SLWNAIDFS

SEQ ID NO: 45 - protein, 9 aa, Homo sapiens
ELQDEVYTL

SEQ ID NO: 46 - protein, 9 aa, Homo sapiens
ELQDEAYTL

SEQ ID NO: 47 - protein, 9 aa, Homo sapiens
LLSIVPCTV

SEQ ID NO: 48 - protein, 9 aa, Homo sapiens
LLSIVLCTV

SEQ ID NO: 49 - protein, 9 aa, Homo sapiens
IIGAGPAEL

SEQ ID NO: 50 - protein, 9 aa, Homo sapiens
IIGAGPAGL

SEQ ID NO: 51 - protein, 9 aa, Homo sapiens
NLFNRYLAL

SEQ ID NO: 52 - protein, 9 aa, Homo sapiens
NLFNRYPAL

SEQ ID NO: 53 - protein, 9 aa, Homo sapiens
WLTRNFYFV

SEQ ID NO: 54 - protein, 9 aa, Homo sapiens
WLTRNLYFV

SEQ ID NO: 55 - protein, 9 aa, Homo sapiens
MLSSVACSL

SEQ ID NO: 56 - protein, 9 aa, Homo sapiens
VLSSVACSL

SEQ ID NO: 57 - protein, 9 aa, Homo sapiens
ILNPADPTL

SEQ ID NO: 58 - protein, 9 aa, Homo sapiens
ILDPADPTL

SEQ ID NO: 59 - protein, 9 aa, Homo sapiens
FLFLVLLFV

SEQ ID NO: 60 - protein, 9 aa, Homo sapiens
FLFSVLLFV

SEQ ID NO: 61 - protein, 9 aa, Homo sapiens
MLLEISENS

SEQ ID NO: 62 - protein, 9 aa, Homo sapiens
MLLEIPENS

SEQ ID NO: 63 - protein, 9 aa, Homo sapiens
LLSIIFFPA

SEQ ID NO: 64 - protein, 9 aa, Homo sapiens
LLSIISFPA

SEQ ID NO: 65 - protein, 9 aa, Homo sapiens
PLANSIWNV

SEQ ID NO: 66 - protein, 9 aa, Homo sapiens
PLANPIWNV

SEQ ID NO: 67 - protein, 9 aa, Homo sapiens
HIVEISTPV

SEQ ID NO: 68 - protein, 9 aa, Homo sapiens
HIVEISTPV

SEQ ID NO: 69 - protein, 9 aa, Homo sapiens
AMFWSVPTV

SEQ ID NO: 70 - protein, 9 aa, Homo sapiens
AMFRSVPTS

SEQ ID NO: 71 - protein, 9 aa, Homo sapiens
CLNEYHLFL

SEQ ID NO: 72 - protein, 9 aa, Homo sapiens
CLNEYHLFF

SEQ ID NO: 73 - protein, 9 aa, Homo sapiens
CLFPQTLAA

SEQ ID NO: 74 - protein, 9 aa, Homo sapiens
CLSPQTLAA

SEQ ID NO: 75 - protein, 9 aa, Homo sapiens
FLNCNSSRL

SEQ ID NO: 76 - protein, 9 aa, Homo sapiens
SLNCNSSRL

SEQ ID NO: 77 - protein, 9 aa, Homo sapiens
SLEGTSFIV

SEQ ID NO: 78 - protein, 9 aa, Homo sapiens
PLEGTSFIV

SEQ ID NO: 79 - protein, 9 aa, Homo sapiens
GMHLLITGL

SEQ ID NO: 80 - protein, 9 aa, Homo sapiens
GMHLLITGP

SEQ ID NO: 81 - protein, 9 aa, Homo sapiens
VLAVSVLAA

SEQ ID NO: 82 - protein, 9 aa, Homo sapiens
VSAVSVLAA

SEQ ID NO: 83 - protein, 9 aa, Homo sapiens
FMLLTQARI

SEQ ID NO: 84 - protein, 9 aa, Homo sapiens
FMLLTQARL

SEQ ID NO: 85 - protein, 9 aa, Homo sapiens
IQYFRNHNV

SEQ ID NO: 86 - protein, 9 aa, Homo sapiens
IQYFKNHNV

SEQ ID NO: 87 - protein, 9 aa, Homo sapiens
SVMIMAFSV

SEQ ID NO: 88 - protein, 9 aa, Homo sapiens
SDMIMAFSV

SEQ ID NO: 89 - protein, 9 aa, Homo sapiens
SISKPLLPV

SEQ ID NO: 90 - protein, 9 aa, Homo sapiens
SIPKPLLPV

SEQ ID NO: 91 - protein, 9 aa, Homo sapiens
IILVAVPHV

SEQ ID NO: 92 - protein, 9 aa, Homo sapiens
IILVAVQHV

SEQ ID NO: 93 - protein, 9 aa, Homo sapiens
LMLESGYIL

SEQ ID NO: 94 - protein, 9 aa, Homo sapiens
LMLESGYIP

SEQ ID NO: 95 - protein, 9 aa, Homo sapiens
AVDDGKLTV

SEQ ID NO: 96 - protein, 9 aa, Homo sapiens
AVDGGKLTV

SEQ ID NO: 97 - protein, 9 aa, Homo sapiens
SLLLLSVSV

SEQ ID NO: 98 - protein, 9 aa, Homo sapiens
SLLLLPVSV

SEQ ID NO: 99 - protein, 9 aa, Homo sapiens
YMAELTVTL

SEQ ID NO: 100 - protein, 9 aa, Homo sapiens
YMAKLTVTL

SEQ ID NO: 101 - protein, 9 aa, Homo sapiens
KLSREIKPV

SEQ ID NO: 102 - protein, 9 aa, Homo sapiens
KLSREIMPV

SEQ ID NO: 103 - protein, 9 aa, Homo sapiens
AVIDAYTEI

SEQ ID NO: 104 - protein, 9 aa, Homo sapiens
AVINAYTEI

SEQ ID NO: 105 - protein, 9 aa, Homo sapiens
VLLRALPVL

SEQ ID NO: 106 - protein, 9 aa, Homo sapiens
VLLRALPVP

SEQ ID NO: 107 - protein, 9 aa, Homo sapiens
HLYASLSRA

SEQ ID NO: 108 - protein, 9 aa, Homo sapiens
HPYASLSRA

SEQ ID NO: 109 - protein, 9 aa, Homo sapiens
FCFKLSHPL

SEQ ID NO: 110 - protein, 9 aa, Homo sapiens
FCFKLFHPL

SEQ ID NO: 111 - protein, 9 aa, Homo sapiens
MLGEQLFPL

SEQ ID NO: 112 - protein, 9 aa, Homo sapiens
MLGERLFPL

SEQ ID NO: 113 - protein, 9 aa, Homo sapiens
FIWGDAPPT

SEQ ID NO: 114 - protein, 9 aa, Homo sapiens
SIWGDAPPT

SEQ ID NO: 115 - protein, 9 aa, Homo sapiens
RLLCPPARA

SEQ ID NO: 116 - protein, 9 aa, Homo sapiens
RPLCPPARA

SEQ ID NO: 117 - protein, 9 aa, Homo sapiens
KLANPLPYT

SEQ ID NO: 118 - protein, 9 aa, Homo sapiens
KLAKPLPYT

SEQ ID NO: 119 - protein, 9 aa, Homo sapiens
IMAHCILDL

SEQ ID NO: 120 - protein, 9 aa, Homo sapiens
IIAHCILDL

SEQ ID NO: 121 - protein, 9 aa, Homo sapiens
SLAETFLET

SEQ ID NO: 122 - protein, 9 aa, Homo sapiens
SLAETFWET

SEQ ID NO: 123 - protein, 9 aa, Homo sapiens
FVFCALLLV

SEQ ID NO: 124 - protein, 9 aa, Homo sapiens
FVFRALLLV

SEQ ID NO: 125 - protein, 9 aa, Homo sapiens
FTQEKWYHV

SEQ ID NO: 126 - protein, 9 aa, Homo sapiens
FTQEEWYHV

SEQ ID NO: 127 - protein, 9 aa, Homo sapiens
VLDGFIPGT

SEQ ID NO: 128 - protein, 9 aa, Homo sapiens
VLDRFIPGT

SEQ ID NO: 129 - protein, 9 aa, Homo sapiens
KLADYLNVL

SEQ ID NO: 130 - protein, 9 aa, Homo sapiens
KLADYLKVL

SEQ ID NO: 131 - protein, 9 aa, Homo sapiens
YSFLFLYRL

SEQ ID NO: 132 - protein, 9 aa, Homo sapiens
YSFLSLYRL

SEQ ID NO: 133 - protein, 9 aa, Homo sapiens
HLGFLSPRV

SEQ ID NO: 134 - protein, 9 aa, Homo sapiens
HLGSLSPRV

SEQ ID NO: 135 - protein, 9 aa, Homo sapiens
RVVFIGVPV

SEQ ID NO: 136 - protein, 9 aa, Homo sapiens
RVVSIGVPV

SEQ ID NO: 137 - protein, 9 aa, Homo sapiens
SMSPGQPPL

SEQ ID NO: 138 - protein, 9 aa, Homo sapiens
SMSPGQPPP

SEQ ID NO: 139 - protein, 10 aa, Homo sapiens
VVDFTESIPV

SEQ ID NO: 140 - protein, 10 aa, Homo sapiens
VVDSTESIPV

SEQ ID NO: 141 - protein, 10 aa, Homo sapiens
RLFGEAPREL

SEQ ID NO: 142 - protein, 10 aa, Homo sapiens
RPFGEAPREL

SEQ ID NO: 143 - protein, 10 aa, Homo sapiens
ALAALGLSGA

SEQ ID NO: 144 - protein, 10 aa, Homo sapiens
ALAALGLPGA

SEQ ID NO: 145 - protein, 10 aa, Homo sapiens
TIPSEIQIFV

SEQ ID NO: 146 - protein, 10 aa, Homo sapiens
TIPSEIQVFV

SEQ ID NO: 147 - protein, 9 aa, Homo sapiens
ILGIFNEFV

SEQ ID NO: 148 - protein, 9 aa, Homo sapiens
ILGISNEFV

SEQ ID NO: 149 - protein, 9 aa, Homo sapiens
FVGALSFSI

SEQ ID NO: 150 - protein, 9 aa, Homo sapiens
FVGVLSFSI

SEQ ID NO: 151 - protein, 9 aa, Homo sapiens
YVLDLANRL

SEQ ID NO: 152 - protein, 9 aa, Homo sapiens
YVLDLADRL

SEQ ID NO: 153 - protein, 9 aa, Homo sapiens
FLFELIPEP

SEQ ID NO: 154 - protein, 9 aa, Homo sapiens
FPFELIPEP

SEQ ID NO: 155 - protein, 9 aa, Homo sapiens
QMIYSAARV

SEQ ID NO: 156 - protein, 9 aa, Homo sapiens
QMIYSAARA

SEQ ID NO: 157 - protein, 9 aa, Homo sapiens
SLVEEQSPA

SEQ ID NO: 158 - protein, 9 aa, Homo sapiens
SPVEEQSPA

SEQ ID NO: 159 - protein, 10 aa, Homo sapiens
MAFPEAPESV

SEQ ID NO: 160 - protein, 10 aa, Homo sapiens
MASPEAPESV

SEQ ID NO: 161 - protein, 10 aa, Homo sapiens
SLVRDVFSSL

SEQ ID NO: 162 - protein, 10 aa, Homo sapiens
SLVRDVVSSL

SEQ ID NO: 163 - protein, 9 aa, Homo sapiens
LATEKSRWS

SEQ ID NO: 164 - protein, 9 aa, Homo sapiens
LATVKSRWS

SEQ ID NO: 165 - protein, 9 aa, Homo sapiens
YLEPGPVTA

SEQ ID NO: 166 - protein, 9 aa, Homo sapiens
IIGAGPAEV

SEQ ID NO: 167 - protein, 9 aa, Homo sapiens
HLYASLSRV

SEQ ID NO: 168 - DNA, 57 bp, Homo sapiens
cagctggagctgatccagacgatagacaacatcgtgttcgtgcctgcaactagtaag

SEQ ID NO: 169 - DNA, 57 bp, Homo sapiens
aaagttttcgctgtcttgctctccattgtgccgtgcacagtgacactttttctcctt

SEQ ID NO: 170 - DNA, 57 bp, Homo sapiens
aacaagagcgtgataattataggagctggcccagcagaagtggcagcagctagacaa

SEQ ID NO: 171 - DNA, 57 bp, Homo sapiens
gagatcgatgctggactgcttagcataatcttttttcctgcttttgcggtagaggat

SEQ ID NO: 172 - DNA, 63 bp, Homo sapiens
gcgctggaagacctggctatgttttggtcagtgcccacagtgacagtcttctacccttctgat

SEQ ID NO: 173 - DNA, 57 bp, Homo sapiens
cctgcagctcagacctgtctcaacgagtatcacctgttcctgcttctcacaggtgcc

SEQ ID NO: 174 - DNA, 57 bp, Homo sapiens
gctgagggaccctccaaaatgataggtaaccatctgtgggtatgtcggagtcgccat

SEQ ID NO: 175 - DNA, 60 bp, Homo sapiens
actggccctatttttaagctcatgaatatccagcaaaagcttatgaaaacaaatctgaag

SEQ ID NO: 176 - DNA, 60 bp, Homo sapiens
tttagcggaaaagattttgtgagcgcactctgcatgtttctcgagggattcaggctgcca

SEQ ID NO: 177 - DNA, 60 bp, Homo sapiens
ctgcccagtcatgcgtgtcttttcccccaaactctggccgctgaggaggagggcgaggtg

SEQ ID NO: 178 - DNA, 60 bp, Homo sapiens
gatgtgccagaggagtttctctataatctgcttacacgcgtctacggagagccacaccgg

SEQ ID NO: 179 - DNA, 60 bp, Homo sapiens
gctggattgcagcggcagctggacaaatgcagcgcattcgtaaatgagatcgaaaccata

SEQ ID NO: 180 - DNA, 60 bp, Homo sapiens
atcctgccactgctgcaactgtcttgcatttctacctacgtgaatgaagtcgtggtcctc

SEQ ID NO: 181 - DNA, 42 bp, Homo sapiens
tccgagatcagaccatacattagcaagtgctgggactatgcc

SEQ ID NO: 182 - DNA, 60 bp, Homo sapiens
acagtgggggacattgtgctgctgcgagcactgcccgtacttcgagcaaaacacgtgaag

SEQ ID NO: 183 - DNA, 60 bp, Homo sapiens
ggcacacgggacacacatctgtatgccagcttgagccgcgcactccaaacacagtgctgc

SEQ ID NO: 184 - DNA, 60 bp, Homo sapiens
caatgctccggtatgatcatggcccactgtatcctcgacttgttgggcagcagcgggccc

SEQ ID NO: 185 - DNA, 60 bp, Homo sapiens
ccagaggacgcacaggcagtgatcgacgcctacaccgagataaacaagaaacattgctgg

SEQ ID NO: 186 - DNA, 60 bp, Homo sapiens
gaatccctggtcgagatcatcctggtagctgttccacatgtcgattacagccttaggtgt

SEQ ID NO: 187 - DNA, 60 bp, Homo sapiens
ggtgccgatgtcgaaaagctcgccaaccctctcccttatacggaatcaagcaaaaccgcg

SEQ ID NO: 188 - DNA, 60 bp, Homo sapiens
ccacaggagcaaaaaatgttgggcgaacaattgttcccgctgattcaggcgatgcacccg

SEQ ID NO: 189 - DNA, 60 bp, Homo sapiens
gtggtgacacacacctatctcgagccgggccccgtgacagcccaggtagttctgcaggcc

SEQ ID NO: 190 - DNA, 60 bp, Homo sapiens
gcttgggatttcgggagcgtgggtggcgtcttcacatctgttggcaaggcagtgcatcag

SEQ ID NO: 191 - protein, 17 aa, Homo sapiens
CASSQDLSGGVYYGYTF

SEQ ID NO: 192 - protein, 14 aa, Homo sapiens
CSTLLAGGGDEQYV

SEQ ID NO: 193 - protein, 14 aa, Homo sapiens
CASSPTGLGETQYF

SEQ ID NO: 194 - protein, 14 aa, Homo sapiens
CSAPPGPLAHTQYF

SEQ ID NO: 195 - protein, 16 aa, Homo sapiens
CASSFKGTGPNQPQHF

SEQ ID NO: 196 - protein, 16 aa, Homo sapiens
CASSFGGPPNTGELFF

SEQ ID NO: 197 - protein, 14 aa, Homo sapiens
CASSIGPVNTEAFF

SEQ ID NO: 198 - protein, 16 aa, Homo sapiens
CASSVAASPSGNTIYF

SEQ ID NO: 199 - protein, 14 aa, Homo sapiens
CASSPYRAGYEQYF

SEQ ID NO: 200 - protein, 14 aa, Homo sapiens
CASSRTGITDTQYF

SEQ ID NO: 201 - protein, 14 aa, Homo sapiens
CASSIASGIYEQYF

SEQ ID NO: 202 - protein, 13 aa, Homo sapiens
CASSISSSEKLFF

SEQ ID NO: 203 - protein, 15 aa, Homo sapiens
CASSLVVGLALEQYF

SEQ ID NO: 204 - protein, 14 aa, Homo sapiens
CASSFWGLSTEAFF

SEQ ID NO: 205 - protein, 12 aa, Homo sapiens
CASSSDLYEQYF

SEQ ID NO: 206 - protein, 15 aa, Homo sapiens
CASSQEVGSGNTIYF

SEQ ID NO: 207 - protein, 15 aa, Homo sapiens
CASSSAGGGGNTIYF

SEQ ID NO: 208 - protein, 13 aa, Homo sapiens
CASSIAGGYEQYV

SEQ ID NO: 209 - protein, 12 aa, Homo sapiens
CSVVGGLLEAFF

SEQ ID NO: 210 - protein, 16 aa, Homo sapiens
CASSSDWGLMNTEAFF

SEQ ID NO: 211 - protein, 17 aa, Homo sapiens
CASSAVDRVTSYNEQFF

SEQ ID NO: 212 - protein, 15 aa, Homo sapiens
CASSLIAGNSDTQYF

SEQ ID NO: 213 - protein, 16 aa, Homo sapiens
CASRLTAGEYQETQYF

SEQ ID NO: 214 - protein, 12 aa, Homo sapiens
CASSLWDYGYTF

SEQ ID NO: 215 - protein, 14 aa, Homo sapiens
CASSLWGVGTEAFF

SEQ ID NO: 216 - protein, 14 aa, Homo sapiens
CASSYFGVNSPLHF

SEQ ID NO: 217 - protein, 16 aa, Homo sapiens
CATSALAGQGRDEQFF

SEQ ID NO: 218 - protein, 14 aa, Homo sapiens
CASSRLAGTDTQYF

SEQ ID NO: 219 - protein, 16 aa, Homo sapiens
CASSFPGYGLNTEAFF

SEQ ID NO: 220 - protein, 15 aa, Homo sapiens
CASSVLAGGLDTQYF

SEQ ID NO: 221 - protein, 16 aa, Homo sapiens
CASSYMLQTFNTEAFF

SEQ ID NO: 222 - protein, 19 aa, Homo sapiens
CASSPGLLAGGSSWETQYF

SEQ ID NO: 223 - protein, 16 aa, Homo sapiens
CASTSTPGQVGQPQHF

SEQ ID NO: 224 - protein, 16 aa, Homo sapiens
CASKGLAGAYTDTQYF

SEQ ID NO: 225 - protein, 12 aa, Homo sapiens
CASSLGGNEQYF

SEQ ID NO: 226 - protein, 15 aa, Homo sapiens
CASSFTAGLNTEAFF

SEQ ID NO: 227 - protein, 15 aa, Homo sapiens
CASSLVWGLGTEAFF

SEQ ID NO: 228 - protein, 12 aa, Homo sapiens
CASSLGLSGESF

SEQ ID NO: 229 - protein, 15 aa, Homo sapiens
CASSKLAGGLDTQYF

SEQ ID NO: 230 - protein, 15 aa, Homo sapiens
CASTHRTGLNTEAFF

SEQ ID NO: 231 - protein, 14 aa, Homo sapiens
CASSIGGQEETQYF

SEQ ID NO: 232 - protein, 15 aa, Homo sapiens
CASSLEIVGETEAFF

SEQ ID NO: 233 - protein, 13 aa, Homo sapiens
CASSISGGYEQYV

SEQ ID NO: 234 - protein, 15 aa, Homo sapiens
CSARTLAGFTDTQYF

SEQ ID NO: 235 - protein, 13 aa, Homo sapiens
CASSDLLTGELFF

SEQ ID NO: 236 - protein, 12 aa, Homo sapiens
CASSSGLAGYLM

SEQ ID NO: 237 - protein, 15 aa, Homo sapiens
CASSHRTTDEETQYF

SEQ ID NO: 238 - protein, 16 aa, Homo sapiens
CASSYPGYGLNTEAFF

SEQ ID NO: 239 - protein, 12 aa, Homo sapiens
CASSLDLYEQYF

SEQ ID NO: 240 - protein, 16 aa, Homo sapiens
CASSWTGFGLNTEAFF

SEQ ID NO: 241 - protein, 15 aa, Homo sapiens
CASSLITGLSYEQYF

SEQ ID NO: 242 - protein, 15 aa, Homo sapiens
CASSTWTGMNTEAFF

SEQ ID NO: 243 - protein, 16 aa, Homo sapiens
CASSELWGAGDNEQFF

SEQ ID NO: 244 - protein, 15 aa, Homo sapiens
CASSFITGLHYEQYF

SEQ ID NO: 245 - protein, 12 aa, Homo sapiens
CSAQQGIQPQHF

SEQ ID NO: 246 - protein, 15 aa, Homo sapiens
CASSLVGGLAETQYF

SEQ ID NO: 247 - protein, 15 aa, Homo sapiens
CASSFSGGLTHEQYV

SEQ ID NO: 248 - protein, 12 aa, Homo sapiens
CASSLGAGEQYF

SEQ ID NO: 249 - protein, 15 aa, Homo sapiens
CASSPIFGLTNEQYF

SEQ ID NO: 250 - protein, 12 aa, Homo sapiens
CASSYFGGEQFF

SEQ ID NO: 251 - protein, 15 aa, Homo sapiens
CASSQDWGLNYEQYF

SEQ ID NO: 252 - protein, 13 aa, Homo sapiens
CASSTSGGYEQYF

SEQ ID NO: 253 - protein, 15 aa, Homo sapiens
CASSRLAGGLDTQYF

SEQ ID NO: 254 - protein, 13 aa, Homo sapiens
CASSGLITDTQYF

SEQ ID NO: 255 - protein, 15 aa, Homo sapiens
CSARELAGFQETQYF

SEQ ID NO: 256 - protein, 11 aa, Homo sapiens
CSPIRGIEQYV

SEQ ID NO: 257 - protein, 14 aa, Homo sapiens
CAIGPQGGFYEQYF

SEQ ID NO: 258 - protein, 17 aa, Homo sapiens
CATSSAILAGVKETQYF

SEQ ID NO: 259 - protein, 15 aa, Homo sapiens
CASSEGVGLAFEQFF

SEQ ID NO: 260 - protein, 13 aa, Homo sapiens
CAIGLAGAYEQYF

SEQ ID NO: 261 - protein, 18 aa, Homo sapiens
CASSSWTGLSLSFYGYTF

SEQ ID NO: 262 - protein, 13 aa, Homo sapiens
CASSEPGTVEAFF

SEQ ID NO: 263 - protein, 12 aa, Homo sapiens
CSVEEGIDEQYF

SEQ ID NO: 264 - protein, 12 aa, Homo sapiens
CASSLGAGEQFF

SEQ ID NO: 265 - protein, 15 aa, Homo sapiens
CASSFQGGTGNTIYF

SEQ ID NO: 266 - protein, 13 aa, Homo sapiens
CASSLALPYEQYF

SEQ ID NO: 267 - protein, 17 aa, Homo sapiens
CASSPTQGLAITGELFF

SEQ ID NO: 268 - protein, 14 aa, Homo sapiens
CASSQTHPPGELFF

SEQ ID NO: 269 - protein, 13 aa, Homo sapiens
CASSISAGYEQYV

SEQ ID NO: 270 - protein, 14 aa, Homo sapiens
CASSVDGAYNEQFF

SEQ ID NO: 271 - protein, 18 aa, Homo sapiens
CAFGVNWDLPHSGNTIYF

SEQ ID NO: 272 - protein, 15 aa, Homo sapiens
CASSFTWGLNTEAFF

SEQ ID NO: 273 - protein, 12 aa, Homo sapiens
CASSYFSYEQYF

SEQ ID NO: 274 - protein, 17 aa, Homo sapiens
CASSSDRGLPSGNTIYF

SEQ ID NO: 275 - protein, 11 aa, Homo sapiens
CSAHEGLEQYF

SEQ ID NO: 276 - protein, 15 aa, Homo sapiens
CASSASWTDYYGYTF

SEQ ID NO: 277 - protein, 14 aa, Homo sapiens
CASSTGTGSYEQYF

SEQ ID NO: 278 - protein, 13 aa, Homo sapiens
CASSLWYNQPQHF

SEQ ID NO: 279 - protein, 17 aa, Homo sapiens
CASSPLAAPGSFETQYF

SEQ ID NO: 280 - protein, 14 aa, Homo sapiens
CASSVDGDYNEQFF

SEQ ID NO: 281 - protein, 16 aa, Homo sapiens
CASSPTPSGLWWELFF

SEQ ID NO: 282 - protein, 15 aa, Homo sapiens
CASSTGTGLNTEAFF

SEQ ID NO: 283 - protein, 17 aa, Homo sapiens
CATSALPGQETTDTQYF

SEQ ID NO: 284 - protein, 16 aa, Homo sapiens
CASSLVGGLSNQPQHF

SEQ ID NO: 285 - protein, 17 aa, Homo sapiens
CASSQQAGGITYNEQFF

SEQ ID NO: 286 - protein, 14 aa, Homo sapiens
CASSYSTAGQPQHF

SEQ ID NO: 287 - protein, 14 aa, Homo sapiens
CASSPTGAGYEQYF

SEQ ID NO: 288 - protein, 14 aa, Homo sapiens
CASSLLSGSTEAFF

SEQ ID NO: 289 - protein, 14 aa, Homo sapiens
CASSYGTSTNEQFF

SEQ ID NO: 290 - protein, 15 aa, Homo sapiens
CASSQGDSGTDTQYF

SEQ ID NO: 291 - protein, 12 aa, Homo sapiens
CASSFSNQPQHF

SEQ ID NO: 292 - protein, 14 aa, Homo sapiens
CASSGGQGTQPQHF

SEQ ID NO: 293 - protein, 14 aa, Homo sapiens
CASSYSGAGQPQHF

SEQ ID NO: 294 - protein, 15 aa, Homo sapiens
CASSLLQGAESPLHF

SEQ ID NO: 295 - protein, 16 aa, Homo sapiens
CASSPQDRGPNYGYTF

SEQ ID NO: 296 - protein, 13 aa, Homo sapiens
CASSFDYSYEQYF

SEQ ID NO: 297 - protein, 12 aa, Homo sapiens
CAAGGVNQPQHF

SEQ ID NO: 298 - protein, 12 aa, Homo sapiens
CASSLLAGELFF

SEQ ID NO: 299 - protein, 13 aa, Homo sapiens
CASSPSSPYEQYF

SEQ ID NO: 300 - protein, 12 aa, Homo sapiens
CASSEGTDTQYF

SEQ ID NO: 301 - protein, 12 aa, Homo sapiens
CASGISNQPQHF

SEQ ID NO: 302 - protein, 18 aa, Homo sapiens
CASSLDPPFDRQNYGYTF

SEQ ID NO: 303 - protein, 15 aa, Homo sapiens
CASSYGDMAYNEQFF

SEQ ID NO: 304 - protein, 16 aa, Homo sapiens
CATMGTGGSLYYGYTF

SEQ ID NO: 305 - protein, 12 aa, Homo sapiens
CASSVSNQPQHF

SEQ ID NO: 306 - protein, 15 aa, Homo sapiens
CASSFTSGGYNEQFF

SEQ ID NO: 307 - protein, 15 aa, Homo sapiens
CASSLYRANTGELFF

SEQ ID NO: 308 - protein, 14 aa, Homo sapiens
CASSLTSLTDTQYF

SEQ ID NO: 309 - protein, 13 aa, Homo sapiens
CASSKSKGSPLHF

SEQ ID NO: 310 - protein, 16 aa, Homo sapiens
CASSLAGQGPNSPLHF

SEQ ID NO: 311 - protein, 14 aa, Homo sapiens
CASSPTGAGQPQHF

SEQ ID NO: 312 - protein, 15 aa, Homo sapiens
CASSSGTSGSDTQYF

SEQ ID NO: 313 - protein, 14 aa, Homo sapiens
CASSFSGPRSPQHF

SEQ ID NO: 314 - protein, 14 aa, Homo sapiens
CASNLQGLDYEQYF

SEQ ID NO: 315 - protein, 14 aa, Homo sapiens
CASSLGQGNQPQHF

SEQ ID NO: 316 - protein, 14 aa, Homo sapiens
CASSFWGANEKLFF

SEQ ID NO: 317 - protein, 15 aa, Homo sapiens
CASSYSVGVNTEAFF

SEQ ID NO: 318 - protein, 15 aa, Homo sapiens
CASRYRAAPNQPQHF

SEQ ID NO: 319 - protein, 17 aa, Homo sapiens
CASSQDAGGVFGNTIYF

SEQ ID NO: 320 - protein, 13 aa, Homo sapiens
CASSLYSNQPQHF

SEQ ID NO: 321 - protein, 12 aa, Homo sapiens
CATAPINSPLHF

SEQ ID NO: 322 - protein, 12 aa, Homo sapiens
CASSPPNQPQHF

SEQ ID NO: 323 - protein, 12 aa, Homo sapiens
CASSFNNQPQHF

SEQ ID NO: 324 - protein, 12 aa, Homo sapiens
CASGVSNQPQHF

SEQ ID NO: 325 - protein, 13 aa, Homo sapiens
CASSYESNYGYTF

SEQ ID NO: 326 - protein, 15 aa, Homo sapiens
CASSLDVATNEKLFF

SEQ ID NO: 327 - protein, 13 aa, Homo sapiens
CSDSSTGGAGFTF

SEQ ID NO: 328 - protein, 17 aa, Homo sapiens
CASSESGGGYRWTEAFF

SEQ ID NO: 329 - protein, 12 aa, Homo sapiens
CASSEGPSGYTF

SEQ ID NO: 330 - protein, 12 aa, Homo sapiens
CASSPGLGEQYF

SEQ ID NO: 331 - protein, 13 aa, Homo sapiens
CASSLEGVYGYTF

SEQ ID NO: 332 - protein, 15 aa, Homo sapiens
CASTIGPGITDTQYF

SEQ ID NO: 333 - protein, 16 aa, Homo sapiens
CASSPRDRGPRSPQHF

SEQ ID NO: 334 - protein, 14 aa, Homo sapiens
CASSRTGAGEKLFF

SEQ ID NO: 335 - protein, 16 aa, Homo sapiens
CASSLGIAGPYNEQFF

SEQ ID NO: 336 - protein, 12 aa, Homo sapiens
CAGGLLNQPQHF

SEQ ID NO: 337 - protein, 14 aa, Homo sapiens
CASSLGQGAQPQHF

SEQ ID NO: 338 - protein, 12 aa, Homo sapiens
CASSPMNTEAFF

SEQ ID NO: 339 - protein, 12 aa, Homo sapiens
CASSLSSHGYTF

SEQ ID NO: 340 - protein, 14 aa, Homo sapiens
CASSFATVGEKLFF

SEQ ID NO: 341 - protein, 14 aa, Homo sapiens
CASTLYTGDNEQFF

SEQ ID NO: 342 - protein, 15 aa, Homo sapiens
CASSYSAGGYYGYTF

SEQ ID NO: 343 - protein, 14 aa, Homo sapiens
CASSYQQGSQPQHF

SEQ ID NO: 344 - protein, 12 aa, Homo sapiens
CASSPLNTEAFF

SEQ ID NO: 345 - protein, 12 aa, Homo sapiens
CASSWSNQPQHF

SEQ ID NO: 346 - protein, 16 aa, Homo sapiens
CASSPVTGGDNSPLHF

SEQ ID NO: 347 - protein, 12 aa, Homo sapiens
CASSSGNYEQYF

SEQ ID NO: 348 - protein, 16 aa, Homo sapiens
CASSLGLSGAYNEQFF

SEQ ID NO: 349 - protein, 13 aa, Homo sapiens
CAWSVASGNEQFF

SEQ ID NO: 350 - protein, 14 aa, Homo sapiens
CASSWGQGGYEQYF

SEQ ID NO: 351 - protein, 14 aa, Homo sapiens
CAWSVGVSNQPQHF

SEQ ID NO: 352 - protein, 13 aa, Homo sapiens
CASSLGQGGELFF

SEQ ID NO: 353 - protein, 12 aa, Homo sapiens
CASSLGNYEQYF

SEQ ID NO: 354 - protein, 15 aa, Homo sapiens
CAWSAGTGGNEKLFF

SEQ ID NO: 355 - protein, 13 aa, Homo sapiens
CAWSVAGGHEQYF

SEQ ID NO: 356 - protein, 13 aa, Homo sapiens
CASSLGQGYEQYF

SEQ ID NO: 357 - protein, 14 aa, Homo sapiens
CASSFGQRETEAFF

SEQ ID NO: 358 - protein, 14 aa, Homo sapiens
CASSQGTGVTEAFF

SEQ ID NO: 359 - protein, 13 aa, Homo sapiens
CASSFGTGYEQYF

SEQ ID NO: 360 - protein, 12 aa, Homo sapiens
CASSLNPDTQYF

SEQ ID NO: 361 - protein, 16 aa, Homo sapiens
CAWSPGQGGTNEKLFF

SEQ ID NO: 362 - protein, 12 aa, Homo sapiens
CAWSAYTGELFF

SEQ ID NO: 363 - protein, 14 aa, Homo sapiens
CAWSVGAGVGEQYF

SEQ ID NO: 364 - protein, 12 aa, Homo sapiens
CAWSGDRPLAFF

SEQ ID NO: 365 - protein, 19 aa, Homo sapiens
CASSVGLSETTALYNEQFF

SEQ ID NO: 366 - protein, 14 aa, Homo sapiens
CASSLEVVQETQYF

SEQ ID NO: 367 - protein, 15 aa, Homo sapiens
CSARDPASWGEKLFF

SEQ ID NO: 368 - protein, 15 aa, Homo sapiens
CASSVAGLQGAEQYF

SEQ ID NO: 369 - protein, 14 aa, Homo sapiens
CASSYEQGSYEQYF

SEQ ID NO: 370 - protein, 16 aa, Homo sapiens
CASSFGPLGMWAEAFF

SEQ ID NO: 371 - protein, 14 aa, Homo sapiens
CASSYLSVQETQYF

SEQ ID NO: 372 - protein, 14 aa, Homo sapiens
CASSLETGYGEQYF

SEQ ID NO: 373 - protein, 15 aa, Homo sapiens
CASSVFGLAGAEQYF

SEQ ID NO: 374 - protein, 16 aa, Homo sapiens
CASSEFGGGSPDTQYF

SEQ ID NO: 375 - protein, 13 aa, Homo sapiens
CASSVYGGAEAFF

SEQ ID NO: 376 - protein, 15 aa, Homo sapiens
CASSTYGLAGETQYF

SEQ ID NO: 377 - protein, 13 aa, Homo sapiens
CSVENRVIYGYTF

SEQ ID NO: 378 - protein, 13 aa, Homo sapiens
CSVEDPTFYGYTF

SEQ ID NO: 379 - protein, 14 aa, Homo sapiens
CASSLGSSGNTIYF

SEQ ID NO: 380 - protein, 15 aa, Homo sapiens
CSVEGQIAGKYGYTF

SEQ ID NO: 381 - protein, 14 aa, Homo sapiens
CASSYGTSGTEQFF

SEQ ID NO: 382 - protein, 16 aa, Homo sapiens
CSVEDGAAKQIYGYTF

SEQ ID NO: 383 - protein, 14 aa, Homo sapiens
CASSVEYSNQPQHF

SEQ ID NO: 384 - protein, 13 aa, Homo sapiens
CSVEDRVNYGYTF

SEQ ID NO: 385 - protein, 15 aa, Homo sapiens
CASSQWSSTNEKLFF

SEQ ID NO: 386 - protein, 15 aa, Homo sapiens
CARNHDRDRLYEQYF

SEQ ID NO: 387 - protein, 14 aa, Homo sapiens
CASSSWGTSDEQYF

SEQ ID NO: 388 - protein, 9 aa, Homo sapiens
LSALTRCEK

SEQ ID NO: 389 - protein, 9 aa, Homo sapiens
LSALTLCEK

SEQ ID NO: 390 - protein, 9 aa, Homo sapiens
LSACNSPSK

SEQ ID NO: 391 - protein, 9 aa, Homo sapiens
LPACNSPSK

SEQ ID NO: 392 - protein, 9 aa, Homo sapiens
KVFEHVGSR

SEQ ID NO: 393 - protein, 9 aa, Homo sapiens
KVSEHVGSR

SEQ ID NO: 394 - protein, 9 aa, Homo sapiens
FISNTVFRK

SEQ ID NO: 395 - protein, 9 aa, Homo sapiens
FMSNTVFRK

SEQ ID NO: 396 - protein, 9 aa, Homo sapiens
HQCCIDNFK

SEQ ID NO: 397 - protein, 9 aa, Homo sapiens
HQCCIDNFK

SEQ ID NO: 398 - protein, 9 aa, Homo sapiens
SSAAGSFGY

SEQ ID NO: 399 - protein, 9 aa, Homo sapiens
SSAAGSCGY

SEQ ID NO: 400 - protein, 9 aa, Homo sapiens
KIYRRQIFK

SEQ ID NO: 401 - protein, 9 aa, Homo sapiens
KIYRRQVFK

SEQ ID NO: 402 - protein, 9 aa, Homo sapiens
LLLLLCTSV

SEQ ID NO: 403 - protein, 9 aa, Homo sapiens
LLLLLCTPV

SEQ ID NO: 404 - protein, 9 aa, Homo sapiens
RLIQGFVLL

SEQ ID NO: 405 - protein, 9 aa, Homo sapiens
RLIQDFVLL

SEQ ID NO: 406 - protein, 9 aa, Homo sapiens
QLIVIFIFV

SEQ ID NO: 407 - protein, 9 aa, Homo sapiens
QLIVIFILV

SEQ ID NO: 408 - protein, 9 aa, Homo sapiens
ALAFITFFL

SEQ ID NO: 409 - protein, 9 aa, Homo sapiens
ALAFITSFL

SEQ ID NO: 410 - protein, 9 aa, Homo sapiens
LLHGFSFYL

SEQ ID NO: 411 - protein, 9 aa, Homo sapiens
LLHGFSFYL

SEQ ID NO: 412 - protein, 9 aa, Homo sapiens
TLFENTPKA

SEQ ID NO: 413 - protein, 9 aa, Homo sapiens
ALFENTPKA

SEQ ID NO: 414 - protein, 9 aa, Homo sapiens
KLNLVCCEL

SEQ ID NO: 415 - protein, 9 aa, Homo sapiens
KLNPVCCEL

SEQ ID NO: 416 - protein, 9 aa, Homo sapiens
YLGMESFIV

SEQ ID NO: 417 - protein, 9 aa, Homo sapiens
HLGMESFIV

SEQ ID NO: 418 - protein, 9 aa, Homo sapiens
YILKYSVFL

SEQ ID NO: 419 - protein, 9 aa, Homo sapiens
YISKYSVFL

SEQ ID NO: 420 - protein, 9 aa, Homo sapiens
VLQEATICV

SEQ ID NO: 421 - protein, 9 aa, Homo sapiens
VLQEATFCV

SEQ ID NO: 422 - protein, 9 aa, Homo sapiens
GLYDKAICI

SEQ ID NO: 423 - protein, 9 aa, Homo sapiens
GLYDKTICI

SEQ ID NO: 424 - protein, 9 aa, Homo sapiens
YLLKIGSQV

SEQ ID NO: 425 - protein, 9 aa, Homo sapiens
YLLKMGSQV

SEQ ID NO: 426 - protein, 9 aa, Homo sapiens
FLGEAWAQV

SEQ ID NO: 427 - protein, 9 aa, Homo sapiens
SLGEAWAQV

SEQ ID NO: 428 - protein, 9 aa, Homo sapiens
SLSSMACCI

SEQ ID NO: 429 - protein, 9 aa, Homo sapiens
SLSSMACCI

SEQ ID NO: 430 - protein, 9 aa, Homo sapiens
SLVVTLWPL

SEQ ID NO: 431 - protein, 9 aa, Homo sapiens
SLVVTLWLL

SEQ ID NO: 432 - protein, 9 aa, Homo sapiens
SMWRFTFYL

SEQ ID NO: 433 - protein, 9 aa, Homo sapiens
SMWRFSFYL

SEQ ID NO: 434 - protein, 9 aa, Homo sapiens
CLLQLCPRL

SEQ ID NO: 435 - protein, 9 aa, Homo sapiens
RLLQLCPRL

SEQ ID NO: 436 - protein, 9 aa, Homo sapiens
SLLRSLENV

SEQ ID NO: 437 - protein, 9 aa, Homo sapiens
SLLRSPENV

SEQ ID NO: 438 - protein, 9 aa, Homo sapiens
FLISILYSA

SEQ ID NO: 439 - protein, 9 aa, Homo sapiens
FLISIPYSA

SEQ ID NO: 440 - protein, 9 aa, Homo sapiens
YLLRWSVPL

SEQ ID NO: 441 - protein, 9 aa, Homo sapiens
YLLKWSVPL

SEQ ID NO: 442 - protein, 9 aa, Homo sapiens
MLTTAIPKV

SEQ ID NO: 443 - protein, 9 aa, Homo sapiens
MLTTAIPKV

SEQ ID NO: 444 - protein, 9 aa, Homo sapiens
HILEILWTL

SEQ ID NO: 445 - protein, 9 aa, Homo sapiens
HILEIPWTL

SEQ ID NO: 446 - protein, 9 aa, Homo sapiens
NMFDILVGL

SEQ ID NO: 447 - protein, 9 aa, Homo sapiens
NVFDILVGL

SEQ ID NO: 448 - protein, 9 aa, Homo sapiens
RMWDFDIFL

SEQ ID NO: 449 - protein, 9 aa, Homo sapiens
GMWDFDIFL

SEQ ID NO: 450 - protein, 9 aa, Homo sapiens
IMSSSLFNL

SEQ ID NO: 451 - protein, 9 aa, Homo sapiens
IMSPSLFNL

SEQ ID NO: 452 - protein, 9 aa, Homo sapiens
RLSDFSEQL

SEQ ID NO: 453 - protein, 9 aa, Homo sapiens
RLSDLSEQL

SEQ ID NO: 454 - protein, 9 aa, Homo sapiens
MPKFKMSSF

SEQ ID NO: 455 - protein, 9 aa, Homo sapiens
MPKFKMPSF

SEQ ID NO: 456 - protein, 9 aa, Homo sapiens
LPSMHRHQI

SEQ ID NO: 457 - protein, 9 aa, Homo sapiens
LPSMYRHQI

SEQ ID NO: 458 - protein, 9 aa, Homo sapiens
LPRAKKLIL

SEQ ID NO: 459 - protein, 9 aa, Homo sapiens
LPRAKELIL

SEQ ID NO: 460 - protein, 9 aa, Homo sapiens
FSNYRRSRL

SEQ ID NO: 461 - protein, 9 aa, Homo sapiens
FPNYRRSRL

SEQ ID NO: 462 - protein, 9 aa, Homo sapiens
QLILRTKAF

SEQ ID NO: 463 - protein, 9 aa, Homo sapiens
QPILRTKAF

SEQ ID NO: 464 - protein, 9 aa, Homo sapiens
YLKYHCNAS

SEQ ID NO: 465 - protein, 9 aa, Homo sapiens
YLKYHYNAS

SEQ ID NO: 466 - protein, 9 aa, Homo sapiens
SLRSHHYSL

SEQ ID NO: 467 - protein, 9 aa, Homo sapiens
SLRSHHYSP

SEQ ID NO: 468 - protein, 9 aa, Homo sapiens
FFSMHFQAF

SEQ ID NO: 469 - protein, 9 aa, Homo sapiens
FFPMHFQAF

SEQ ID NO: 470 - protein, 9 aa, Homo sapiens
LFRYKSISV

SEQ ID NO: 471 - protein, 9 aa, Homo sapiens
LFRYKSIPV

SEQ ID NO: 472 - protein, 9 aa, Homo sapiens
NLRYFAKSL

SEQ ID NO: 473 - protein, 9 aa, Homo sapiens
NLRYSAKSL

SEQ ID NO: 474 - DNA, 12 bp, Artificial Sequence
  synthetic dna
ctttgtgtttga